(19) United States
(12) Patent Application Publication
(10) Pub. No.: US 2008/0145899 A1
(43) Pub. Date: Jun. 19, 2008

(54) Production of Oligosaccharides By Microorganisms

(75) Inventors: Karl Johnson, Hatboro, PA (US); Noel J. Byrne, Jenkintown, PA (US); Shawn DeFrees, North Wales, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neose Technologies Inc, Horsham, PA (US)

(21) Appl. No.: 11/661,621

(22) PCT Filed: Sep. 19, 2005
(86) PCT No.: PCT/US2005/033532

§ 371 Date: Aug. 31, 2007

Related U.S. Application Data
(60) Provisional application No. 60/610,704, filed on Sep. 17, 2004.

(51) Int. Cl. C12P 19/18; C12P 19/12

(52) U.S. Cl. 435/97; 435/100

(57) ABSTRACT

The present invention relates to the enzymatic synthesis of oligosaccharides, including sialylated product saccharides. In particular, it relates to the use of recombinant cells to take up low cost precursors such as glucose, pyruvate and N-actyl-glucosamine, and to synthesize activated sugar moieties that are used in oligosaccharide synthesis. The methods make possible the synthesis of many oligosaccharides using microorganisms and readily available, relatively inexpensive starting materials.

### CROSS-REFERENCES TO RELATED APPLICATIONS

This application claims the benefit of U.S. Provisional Application No. 60/610,704, filed Sep. 17, 2004, which is herein incorporated by reference for all purposes.

### FIELD OF THE INVENTION

The present invention relates to the enzymatic synthesis of oligosaccharides, including sialylated product saccharides. In particular, it relates to the use of recombinant cells to take up low cost precursors such as glucose, pyruvate and N-actylglucosamine, and to synthesize activated sugar moieties that are used in oligosaccharide synthesis. The methods make possible the synthesis of many oligosaccharides using microorganisms and readily available, relatively inexpensive starting materials.

### BACKGROUND OF THE INVENTION

Oligosaccharides, are commercially important molecules. Their commercial scale production, however, is often complicated by the cost and difficulty in obtaining reactants that are used in the enzymatic and chemical synthesis of donor sugar moieties or activated donor sugar moieties. Sialylated product saccharides are of particular interest. In particular, nucleotide sugars, such as CMP-sialic acid, that are used as substrates for many sialyltransferases are expensive or difficult to obtain.

The use of cell-based systems for oligosaccharide synthesis has been described. Endo et al. ((1999) Carbohydrate Res. 316: 179-183; describe the use of a coupling of a combination of different cell types, each producing a different glycosyltransferase nucleotide sugar, to produce N-acetyllactosamine. See also, Koizumi et al. Nature Biotechnology 16: 847-850 (1998); Ringenberg et al., Glycobiology 11:533-539 (2001); Dumon et al., Glycoconjugate J. 18, 465-474 (2001); Priem et al, Glycobiology 12:235-240 (2002); and Antoine et al., ChemBioChem 4:406-412 (2003). These methods, however, require multiple cell types for each reaction, one to produce the transferase and the other to produce the nucleotide sugar or require relatively expensive starting materials, e.g., sialic acid.

Improved methods for enzymatic synthesis of oligosaccharides, and precursors used in these syntheses, would advance the production of a number of beneficial compounds. The present invention fulfills these and other needs.

### BRIEF SUMMARY OF THE INVENTION

In one aspect, the present invention provides methods of producing oligosaccharides by fermentative growth of microorganisms. The microorganisms are grown in a culture medium comprising a glucose moiety and the microorganism comprises a heterologous galactosyltransferase protein with galactosyltransferase activity that catalyzes the transfer of a galactose moiety from an activated galactose molecule to the glucose moiety to form a disaccharide that is the product oligosaccharide. In further embodiments, additional sugars are added to the disaccharide to form different product oligosaccharides.

In one embodiment, the microorganism also comprises a heterologous enzymatic system for synthesizing an activated galactose moiety from glucose.

In one embodiment, the product oligosaccharide is lactose.

In one embodiment, the glucose moiety is glucose. In a further, embodiment the glucose moiety comprises a ractive group, such as fluoride. In a further embodiment, the glucose moiety is N-acetylglucosamine (GlcNAc).

The galactosyltransferase activity can be e.g., β1,3-galactosyltransferase activity or β1,4-galactosyltransferase activity.

In one embodiment, the oligosaccharide is isolated from the microorganism, or from the culture medium, or from both.

In one embodiment, the microorganism also includes a second heterologous glycosyltransferase that catalyzes transfer of an activated sugar moiety to the disaccharide. In a further embodiment, the second heterologous glycosyltransferase is a sialyltransferase. In another embodiment, the microorganism also includes at least one of the following heterologous glycosyltransferases: fucosyltransferase, N-acetylglucosaminyl (GlcNAc) transferase, an N-acetylgalactosaminyl (GalNAc) transferase, and an α1,4-galactosyltransferase.

In one embodiment, the culture medium further comprises N-acetylglucosamine (GlcNAc), and the microorganism further comprises an enzymatic system for synthesizing sialic acid from GlcNAc, a CMP-sialic acid synthase polypeptide, and a sialyltransferase polypeptide, wherein culture takes places under conditions suitable for synthesizing an activated sialic acid molecule and wherein the sialyltransferase polypeptide catalyzes the transfer of a sialic acid moiety from the activated sialic acid molecule to the disaccharide comprising a glucose moiety to produce the oligosaccharide. In a further embodiment, the disaccharide comprising a glucose moiety is lactose.

In one aspect the invention provides a method of producing a sialylated product saccharide, by growing a microorganism in a culture media comprising N-acetylglucosamine (GlcNAc) and an acceptor substrate. The microorganism comprises an enzymatic system for synthesizing sialic acid from N-acetylglucosamine, a CMP-sialic acid synthase polypeptide, and a sialyltransferase polypeptide. Growth of the microorganism takes places under conditions suitable for synthesizing an activated sialic acid molecule, e.g., CMP-sialic acid. The sialyltransferase polypeptide then catalyzes the transfer of a sialic acid moiety from the activated sialic acid molecule to the acceptor substrate to produce the sialylated product saccharide. In one embodiment, the sialylated product saccharide is isolated from the microorganism or the culture medium or from both.

In one embodiment, the enzymatic system for synthesizing sialic acid comprises an N-acetylglucosamine (GlcNAc) epimerase polypeptide and an N-acetyl neuraminic acid condensing polypeptide. The GlcNAc epimerase polypeptide can be a heterologous protein. In a further embodiment, the heterologous GlcNAc epimerase polypeptide is a bacterial protein, for example, the Neisseria SiaA protein. The N-acetyl neuraminic acid condensing polypeptide can a heterologous protein. In a further embodiment, the heterologous N-acetyl neuraminic acid condensing polypeptide is a bacterial protein., for example, the Neisseria SiaC protein.

In one embodiment, the enzymatic system for synthesizing sialic acid comprises a UDP-GlcNAc epimerase polypeptide and a sialate synthase polypeptide. The UDP-GlcNAc epimerase polypeptide can be heterologous protein. In a further embodiment, the heterologous UDP-GlcNAc epimerase polypeptide is a bacterial protein, for example, the NeuC protein from E. coli K1. The sialate synthase polypeptide can be heterologous protein. In a further embodiment, the heterologous sialate synthase polypeptide is a bacterial protein, for example the NeuA protein from E. coli K1.

In one embodiment, the CMP-sialic acid synthase polypeptide is a heterologous protein. In a further embodiment, the heterologous CMP-sialic acid synthase polypeptide is a bacterial protein, for example, the CMP-sialic acid synthase polypeptide is from Neisseria.

In one embodiment, the sialyltransferase polypeptide is a heterologous protein. In a further embodiment, the heterologous sialyltransferase polypeptide is a bacterial protein, such as a sialyltransferase from Neisseria or from Campylobacter. In another embodiment the sialyltransferase polypeptide has α-2,3-sialyltransferase activity.

In one embodiment, the microorganism is a bacterium. In a further embodiment, the bacterium is Escherichia coli.

In one embodiment, the acceptor saccharide is a monosaccharide selected from the group consisting of glucose, galactose, and mannose.

In one embodiment, the acceptor substrate is a disaccharide, such as lactose. In a further embodiment, the microorganism is unable to break down the disaccharide into component sugars. In another embodiment, the acceptor substrate is lactose and the sialylated product sugar is sialylactose.

In a further, embodiment the acceptor substrate comprises a reactive group, such as fluoride.

In one embodiment, the microorganism comprises a protein with α-2,8-sialyltransferase activity. In a further embodiment, the sialyltransferase polypeptide has α-2,3-sialyltransferase activity and α-2,8-sialyltransferase activity.

In one embodiment, the sialyltransferase polypeptide is a heterologous protein and the CMP-N-acetyl neuraminic acid synthetase polypeptide is a heterologous protein. In a further embodiment, the heterologous sialyltransferase polypeptide and the heterologous CMP-N-acetyl neuraminic acid synthetase polypeptide are fused to form a single fusion protein. The heterologous CMP-N-acetyl neuraminic acid synthetase polypeptide can be, e.g., a Neisseria protein. The heterologous sialyltransferase polypeptide can be e.g., a Neisseria protein or a Campylobacter protein.

In one embodiment, the culture medium comprises pyruvate.

In one embodiment, the sialylated product is produced on a commercial scale. For example, greater than 50 grams of the sialylated product is produced.

In one embodiment, the bacterium further comprises a heterologous CTP synthetase polypeptide. In a further embodiment, the heterologous CTP synthetase polypeptide is a pyrG protein.

In one embodiment, the GlcNAc epimerase polypeptide is a heterologous protein, the N-acetyl neuraminic acid condensing polypeptide is a heterologous protein, the CMP-sialic acid synthase polypeptide is a heterologous protein, and the sialyltransferase polypeptide is a heterologous protein. For example, the GlcNAc epimerase polypeptide is a SiaA protein from Neisseria, the N-acetyl neuraminic acid condensing polypeptide is a SiaC protein from Neisseria, the CMP-sialic acid synthase polypeptide is from Neisseria, and the sialyltransferase polypeptide is from Neisseria. In a preferred embodiment, the acceptor saccharide is lactose and the sialylated product sugar is sialylactose. The microorganism can include an expression vector that comprises a nucleic acid encoding the heterologous N-acetylglucosamine epimerase polypeptide, the heterologous N-acetyl neuraminic acid condensing polypeptide, the heterologous nucleotide pyrophosphorylase polypeptide, or the heterologous sialyltransferase polypeptide. Alternatively, the expression vector can include a first nucleic acid encoding the heterologous N-acetylglucosamine epimerase polypeptide, a second nucleic acid encoding the heterologous N-acetyl neuraminic acid condensing polypeptide, a third nucleic acid encoding the heterologous nucleotide pyrophosphorylase polypeptide, and a fourth nucleic acid encoding the heterologous sialyltransferase polypeptide.

In another embodiment, the expression vector comprises an inducible promoter.

### DETAILED DESCRIPTION OF THE INVENTION

### I. Introduction

The present invention provides cell-based methods for enzymatically synthesizing oligosaccharides. Also provided are reaction mixtures, expression cassettes, and recombinant cells that are useful in methods for synthesizing oligosaccharides. In some embodiments, the invention employs cells that produce recombinant glycosyltransferases, and that also produce an activated donor substrate molecule for the recombinant sialyltransferase. In presently preferred embodiments, the cells are grown in medium that includes a precursor of the activated donor substrate molecule and an acceptor saccharide or a precursor of the acceptor saccharide. Production of the oligosaccharide occurs during fermentative growth of the cells, as the precursors are taken up by the cells and metabolized to form first the activated donor substrate molecule, the acceptor saccharide, any required intermediate molecules, and finally the desired oligosaccharide. The invention is useful for producing a wide range of product saccharides, including oligosaccharides, polysaccharides, and sugar containing headgroups from lipooligosaccharides, lipopolysaccharides, gangliosides and other glycolipids.

One example of a low cost precursor is glucose. In some embodiments, this disclosure provides methods of making a desired oligosaccharide by fermentative growth of recombinant host bacterium in a medium that comprises glucose. Glucose can serve as a precursor of a donor sugar, e.g., galactose, N-acetylglucosamine (GlcNAc), N-acetylgalactosamine (GalNAc); as a precursor of an activated sugar moiety, e.g., UDP-glucose, UDP-galactose, UDP-GlcNAc, UDP-GalNAc; or as a precursor for an acceptor saccharide for e.g., oligosaccharides such as lactose or oligosaccharides that comprise lactose. Appropriate heterologous proteins are expressed in the host bacterium to allow or to enhance production of a donor sugar or an activated donor sugar and heterologous glycosyltransferases are expressed to produce the oligosaccharide product.

In one embodiment, the host cell is engineered so that glucose is not metabolized. For example, a glucokinase enzyme can be inactivated in the host cell. This inactivation preferably prevents synthesis of glucose-6-phosphate, thereby preventing use of glucose in a glycolytic cycle to allow diversion of glucose into metabolic pathways described herein. The host cell can then be grown in a medium comprising an alternative carbon source, such as glycerol or an amino acid for growth, as well as comprising glucose as the metabolic precursor.

In general, the sialylated product saccharides are produced by growing a microorganism that comprises an enzymatic system for synthesizing sialic acid, a CMP-sialic acid synthase polypeptide, and a sialyltransferase polypeptide in the presence of a precursor of sialic acid and an acceptor saccharide, under conditions such that an activated sialic acid molecule is synthesized and transfer of the sialic acid moiety from the activated sialic acid molecule is catalyzed by the sialyltransferase to produce the sialylated product saccharide. Also provided by the invention are recombinant cells that can be used in the methods, as well as reaction mixtures that include the recombinant cells and are useful for producing the product sugars.

One advantage of the present invention is that the need to supply expensive starting materials, e.g., sialic acid or CMP-sialic acid, is eliminated. Thus, through the use of cells that produce a particular sialyltransferase, but that also can synthesize the activated sialic acid donor from inexpensive starting materials, e.g., GlcNAc, one can achieve highly efficient, rapid, and relatively low cost synthesis of a desired sialylated product saccharide. Sialylated saccharides produced using the methods of the invention find many uses, including, for example, diagnostic and therapeutic uses, as foodstuffs, and the like.

This disclosure also provides methods of producing fucosylated oligosaccharides by using appropriately constructed host microorganisms that are grown in the presence of precursor molecules as described above. For example, in some embodiments, fucose is synthesized from glucose or mannose and added to an acceptor saccharide that is also synthesized from glucose using heterologous enzymes. In other embodiments, fucose is synthesized from glucose or mannose and added to a sialylated oligosaccharide as described above.

### II. Definitions

The cells and methods of the invention are useful for producing a sialylated product, generally by transferring a sialic acid moiety from a donor substrate to an acceptor molecule. The cells and methods of the invention are also useful for producing a sialylated product sugar comprising additional sugar residues, generally by transferring a additional monosaccharide or a sulfate groups from a donor substrate to an acceptor molecule. The addition generally takes place at the non-reducing end of a monosaccharide, disaccharide, oligosaccharide, or a carbohydrate moiety on a glycolipid or glycoprotein, e.g., a biomolecule. Biomolecules as defined here include but are not limited to biologically significant molecules such as carbohydrates, proteins (e.g., glycoproteins), and lipids (e.g., glycolipids, phospholipids, sphingolipids and gangliosides).

The following abbreviations are used herein:
Ara=arabinosyl;
Fru=fructosyl;
Fuc=fucosyl;
Gal=galactosyl;
GalNAc=N-acetylgalactosaminyl;
Glc=glucosyl;
GlcNAc=N-acetylglucosaminyl;
Man=mannosyl; and
NeuAc=sialyl (N-acetylneuraminyl).

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

An “acceptor substrate” or an “acceptor saccharide” for a glycosyltransferase is an oligosaccharide moiety that can act as an acceptor for a particular glycosyltransferase. When the acceptor substrate is contacted with the corresponding glycosyltransferase and sugar donor substrate, and other necessary reaction mixture components, and the reaction mixture is incubated for a sufficient period of time, the glycosyltransferase transfers sugar residues from the sugar donor substrate to the acceptor substrate. The acceptor substrate will often vary for different types of a particular glycosyltransferase. For example, the acceptor substrate for a mammalian galactoside 2-L-fucosyltransferase (α1,2-fucosyltransferase) will include a Galβ1,4-GlcNAc-R at a non-reducing terminus of an oligosaccharide; this fucosyltransferase attaches a fucose residue to the Gal via an α1,2 linkage. Terminal Galβ1,4-GlcNAc-R and Galβ1,3-GlcNAc-R and sialylated analogs thereof are acceptor substrates for α1,3 and α1,4-fucosyltransferases, respectively. These enzymes, however, attach the fucose residue to the GlcNAc residue of the acceptor substrate. Glucose is also an acceptor substrate. For example, galactose can be added to glucose to form lactose through the enzymatic activity of a β1,4-galactosyltransferase. Accordingly, the term “acceptor substrate” is taken in context with the particular glycosyltransferase of interest for a particular application. Acceptor substrates for additional glycosyltransferases, are described herein.

A “donor substrate” for glycosyltransferases is an activated nucleotide sugar. Such activated sugars generally consist of uridine, guanosine, and cytidine monophosphate derivatives of the sugars (UMP, GMP and CMP, respectively) or diphosphate derivatives of the sugars (UDP, GDP and CDP, respectively) in which the nucleoside monophosphate or diphosphate serves as a leaving group. For example, a donor substrate for fucosyltransferases is GDP-fucose. Donor substrates for sialyltransferases, for example, are activated sugar nucleotides comprising the desired sialic acid. For instance, in the case of NeuAc, the activated sugar is CMP-NeuAc. Other donor substrates include e.g., GDP mannose, UDP-galactose, UDP-N-acetylgalactosamine, UDP-N-acetylglucosamine, UDP-glucose, UDP-glucorionic acid, and UDP-xylose. Bacterial, plant, and fungal systems can sometimes use other activated nucleotide sugars.

A “glucose moiety” refers to a molecule that includes glucose or that can be derived from glucose. Glucose moieties are usually monosaccharides, e.g., glucose or GlcNAc. In preferred embodiments, glucose moieties are components of growth medium and are taken up by a microorganism to serve as precursors of e.g., donor substrates, or acceptor substrates. Glucose moiety also includes a glucose molecule that comprises a reactive functional group. In some embodiments glucose moiety includes, e.g., glycosides (alpha or beta), N, S, or O; glucosamine; deoxy-glucosides or the like.

The term “PEG” refers to poly(ethylene glycol). PEG is an exemplary polymer that has been conjugated to peptides. The use of PEG to derivatize peptide therapeutics has been demonstrated to reduce the immunogenicity of the peptides and prolong the clearance time from the circulation. For example, U.S. Pat. No. 4,179,337 (Davis et al.) concerns non-immunogenic peptides, such as enzymes and peptide hormones coupled to polyethylene glycol (PEG) or polypropylene glycol. Between 10 and 100 moles of polymer are used per mole peptide and at least 15% of the physiological activity is maintained.

A “sialylated product saccharide” refers an oligosaccharide, polysaccharide (e.g., heparin, carragenin, and the like) or a carbohydrate moiety, either unconjugated or conjugated to a glycolipid or glycoprotein, e.g., a biomolecule, that includes a sialic acid moiety. Any of the above sialic acid moieties can be used as well as PEGylated sialic acid derivatives. In some embodiments other sugar moieties, e.g., fucose, galactose, glucose, GalNAc, or GluNAc, are also added to the acceptor substrate to produce the sialylated product saccharide. Examples of sialylated product saccharides include, e.g., sialylactose and oligosaccharides disclosed in Tables 1, 4, and 5.

A “fucosylated product saccharide” refers an oligosaccharide, polysaccharide (e.g., heparin, carragenin, and the like) or a carbohydrate moiety, either unconjugated or conjugated to a glycolipid or glycoprotein, e.g., a biomolecule, that includes a fucose moiety. In some embodiments other sugar moieties, e.g., sialic acid, galactose, glucose, GalNAc, or GluNAc, are also added to the acceptor substrate to produce the fucosylated product saccharide. Examples of sialylated product saccharides include, e.g., fucosylated oligosaccharides disclosed in Table 1, 4, and 5.

An “enzymatic system for synthesizing sialic acid from N-acetylglucosamine (GlcNac)” refers to an enzymatic system that converts GlcNAc to sialic acid or N-acetyl neuraminic acid (NANA). Those of skill are aware that more than one pathway exists to convert GlcNAc to sialic acid and that a variety of enzymes can be combined to perform the conversion. For example, in Neisseria, GlcNAc is converted to sialic acid through the actions of at least two enzymes, a GlcNAc epimerase (the SiaA protein, Accession Number M95053 region: 174.1307) and an N-acetyl neuraminic acid (NANA) condensing polypeptide (the SiaC protein, Accession Number M95053 region: 1998.3047). The SiaC protein condenses N-acetyl-D-mannosamine and pyruvate to form NANA. In E. coli K12, for example, UDP-GlcNAc is converted to N-acetyl-D-mannosamine (ManNAc) by UDP-GlcNAc epimerase (the NeuC protein, Accession number M84026). The NeuB gene product (a sialate synthase protein, Accession number AAC43302, encoded by Accession number U05248, region 723-1763) condenses ManNAc and phosphoenol pyruvate to form NANA, which is converted to CMP-NANA by the NeuA gene product (a CMP-sialate synthase protein, Accession number J05023). See, e.g., Ringenberg et al., Glycobiology 11:533-539 (2001). While specific enzymes are listed, those of skill will recognize that other enzymes from different organisms can be used in an enzymatic system for synthesizing sialic acid from GlcNac. In many organisms, sialic acid synthesis proteins are encoded by nucleic acids at localized regions of the chromosomes, e.g., operons. Where exogenous enzymatic systems for synthesizing sialic acid from GlcNac are used, the sialic acid synthetic nucleic acids can be transformed into a microorganism individually or as part of an operon. In some embodiments, an enzymatic system for synthesizing an activated fucose molecule is used to synthesize oligosaccharides that include both sialic acid and fucose residues. As above, nucleic acids encoding individual enzymes for synthesis of activated fucose can be used or appropriate combinations of fucose synthesizing enzymes, e.g., a fucose operon can be expressed in the cells of the invention.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right. All oligosaccharides described herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3, 2→3, or (2,3). Each saccharide is a pyranose or furanose.

The term “contacting” is used herein interchangeably with the following: combined with, added to, mixed with, passed over, incubated with, flowed over, etc.

Much of the nomenclature and general laboratory procedures required in this application can be found in Sambrook, et al, Molecular Cloning: A Laboratory Manual (2nd Ed.), Vol. 1-3, Cold Spring Harbor Laboratory, Cold Spring Harbor, N.Y., 1989. The manual is hereinafter referred to as “Sambrook et al.”

A “growth medium” refers to any liquid, semi-solid or solid media that can be used to support the growth of a microorganism of the invention. In some embodiments, the microorganism is a bacteria, e.g., E. colt. Media for growing microorganisms are well known, see, e.g., Sambrook et al. and Current Protocols in Molecular Biology, F. M. Ausubel et al., eds., Current Protocols, a joint venture between Greene Publishing Associates, Inc. and John Wiley & Sons, Inc., (1998 Supplement) (Ausubel). Medium can be rich medium, e.g., Luria broth or terrific broth, or synthetic or semi-synthetic medium, e.g., M9 medium. In some preferred embodiments the growth medium comprises glucose. In other preferred embodiments, the growth medium comprises a precursor of sialic acid, e.g., N-acetylglucosamine (GlcNAc), phosphenolpyruvate, or pyruvate. Other growth medium components encompassed by this disclosure include e.g., other acceptor substrates such as lactose, and other precursors of donor substrates, such as glucose, N-acetylgalactosamine (GalNAc), fucose, mannose, or galactose.

“Commercial scale” refers to gram scale production of a sialylated product saccharide in a single reaction. In preferred embodiments, commercial scale refers to production of greater than about 50, 75, 80, 90 or 100, 125, 150, 175, or 200 grams.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof. The terms nucleic acid, “nucleic acid sequence”, and “polynucleotide” are used interchangeably herein.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant nucleic acid” refers to a nucleic acid that was artificially constructed (e.g., formed by linking two naturally-occurring or synthetic nucleic acid fragments). This term also applies to nucleic acids that are produced by replication or transcription of a nucleic acid that was artificially constructed. A “recombinant polypeptide” is expressed by transcription of a recombinant nucleic acid (i.e., a nucleic acid that is not native to the cell or that has been modified from its naturally occurring form), followed by translation of the resulting transcript.

A “heterologous polynucleotide” or a “heterologous nucleic acid”, as used herein, is one that originates from a source foreign to the particular host cell, or, if from the same source, is modified from its original form. Thus, a heterologous glycosyltransferase gene in a prokaryotic host cell includes a glycosyltransferase gene that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette. When more than one heterologous protein is expressed in a microorganism, the genes encoding the proteins can be expressed on a single expression cassette or on multiple expression cassettes that are compatible and can be maintained in the same cell. As used herein, expression cassette also encompasses nucleic acid constructs that are inserted into the chromosome of the host microorganism. Those of skill are aware that insertion of a nucleic acid into a chromosome can occur, e.g., by homologous recombination. An expression cassette can be constructed for production of more than one protein. The proteins can be regulated by a single promoter sequence, as for example, an operon. Or multiple proteins can be encoded by nucleic acids with individual promoters and ribosome binding sites.

A “fusion glycosyltransferase polypeptide” of the invention is a polypeptide that contains a glycosyltransferase catalytic domain and a second catalytic domain from an accessory enzyme (e.g., a CMP-Neu5Ac synthetase or a UDP-Glucose 4′ epimerase (galE)). The fusion polypeptide is capable of catalyzing the synthesis of a sugar nucleotide (e.g., CMP-NeuAc or UDP-Gal) as well as the transfer of the sugar residue from the sugar nucleotide to an acceptor molecule. Typically, the catalytic domains of the fusion polypeptides will be at least substantially identical to those of glycosyltransferases and fusion proteins from which the catalytic domains are derived. In some embodiments, the a CMP-sialic acid synthase polypeptide and the sialyltransferase polypeptide are fused to form a single polypeptide. Many sialyltransferase enzymes are known to those of skill and can be used in the methods of the invention. For example, a fusion between a Neisseria CMP-sialic acid synthase polypeptide and a Neisseria sialyltransferase protein is described in, e.g., WO99/31224 and Gilbert et al., Nat. Biotechnol. 16:769-72 (1998), both of which are herein incorporated by reference for all purposes. Other fusions can be used in the invention, for example, between a Neisseria CMP-sialic acid synthase polypeptide and a Campylobacter sialyltransferase, also disclosed in WO99/31224.

An “accessory enzyme,” as referred to herein, is an enzyme that is involved in catalyzing a reaction that, for example, forms a substrate or other reactant for a glycosyltransferase reaction. An accessory enzyme can, for example, catalyze the formation of a nucleotide sugar that is used as a sugar donor moiety by a glycosyltransferase. An accessory enzyme can also be one that is used in the generation of a nucleotide triphosphate that is required for formation of a nucleotide sugar, or in the generation of the sugar which is incorporated into the nucleotide sugar.

A “catalytic domain” refers to a portion of an enzyme that is sufficient to catalyze an enzymatic reaction that is normally carried out by the enzyme. For example, a catalytic domain of a sialyltransferase will include a sufficient portion of the sialyltransferase to transfer a sialic acid residue from a sugar donor to an acceptor saccharide. A catalytic domain can include an entire enzyme, a subsequence thereof, or can include additional amino acid sequences that are not attached to the enzyme or subsequence as found in nature.

The term “isolated” refers to material that is substantially or essentially free from components which interfere with the activity biological molecule. For cells, saccharides, nucleic acids, and polypeptides of the invention, the term “isolated” refers to material that is substantially or essentially free from components which normally accompany the material as found in its native state. Typically, isolated saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., sialylated products, purity can be determined using, e.g., thin layer chromatography, HPLC, or mass spectroscopy.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80% or 85%, most preferably at least 90%, 91%, 92%, 93%, 94%, 95%, 96%, 97%, 98%, or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to” or “specifically immunoreactive with”, when referring to an antibody refers to a binding reaction which is determinative of the presence of the protein or other antigen in the presence of a heterogeneous population of proteins, saccharides, and other biologics. Thus, under designated immunoassay conditions, the specified antibodies bind preferentially to a particular antigen and do not bind in a significant amount to other molecules present in the sample. Specific binding to an antigen under such conditions requires an antibody that is selected for its specificity for a particular antigen. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989). In some embodiments, glucose moieties, as described herein, comprise reactive functional groups.

As used herein, “linking member” refers to a covalent chemical bond that includes at least one heteroatom. Exemplary linking members include —C(O)NH—, —C(O)O—, —NH—, —S—, —O—, and the like.

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO, saccharides, lectins, receptors, ligand for receptors, proteins such as BSA and the like. The targeting group can also be a small molecule, a term that is intended to include both non-peptides and peptides.

The symbol [symbol] whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl,” and “alkylene.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

### III. Donor Substrates and Acceptor Substrates

Suitable donor substrates used by glycosyltransferases in the methods of the invention include, but are not limited to, UDP-Glc, UDP-GlcNAc, UDP-Gal, UDP-GalNAc, GDP-Man, GDP-Fuc, UDP-GlcUA, and CMP-sialic acid and other activated sialic acid moieties. Guo et al., Applied Biochem. and Biotech. 68: 1-20 (1997). In some embodiments, donor substrates are synthesized in the cell from precursors that were components of the medium. Precursors of donor substrates include, e.g., pyruvate, glcNAc, and other monosaccharides.

Suitable acceptor substrates have a terminal sugar residue for addition of a desired sugar residue in a desired linkage. In some embodiments, an acceptor substrate is a monosaccharide, e.g., glucose or N-acetylglucosamine. To facilitate synthesis of multi-residues oligosaccharides, more than one acceptor saccharide can be present in a cell as the final oligosaccharide product is synthesized sequentially by multiple glycosyltransferases in the cell. Thus, acceptor saccharides can be synthesized within the cell or can be components of growth medium that are taken up by the cell and acted on by an appropriate sialyltransferase. Examples of an acceptor saccharide that can be synthesized in a cell are, e.g., lactose and sialylactose. See, e.g., Example 4b and c. Examples of acceptor saccharides that can be medium components and that are taken up by cells and processed include, e.g., glucose and lactose. See, e.g., Examples 1-3 and 4a.

In some embodiments, an acceptor substrate or a donor substrate comprises a reactive compound, e.g. an activated leaving group. In some embodiments, an acceptor substrate or a donor substrate comprises one or more acyl groups.

For some sialylated product saccharides, acceptor substrates include a terminal galactose residue for addition of a sialic acid residue by an α2,3 linkage. For addition of a sialic acid residue in an β2,8 linkage, a second sialic acid residue is linked to a first sialic acid by an α2,8 linkage. Sialylated product saccharides can also comprise a sialic acid residue in an α2,6 linkage. Examples of suitable acceptors include a terminal Gal that is linked to GlcNAc or Glc by a β1,4 linkage, and a terminal Gal that is β1,3-linked to either GlcNAc or GalNAc. Suitable acceptors, include, for example, galactosyl acceptors such as Galβ1,4GlcNAc, Galβ1,4GalNAc, Galβ1,3GalNAc, lacto-N-tetraose, Galβ1,3GlcNAc, Galβ1,3Ara, Galβ1,6GlcNAc, Galβ1,4Glc (lactose), and other acceptors known to those of skill in the art (see, e.g., Paulson et al., J. Biol. Chem. 253: 5617-5624 (1978)). The terminal residue to which the sialic acid is attached can itself be attached to, for example, H, a saccharide, oligosaccharide, or an aglycone group having at least one carbohydrate atom. In some embodiments, the acceptor residue is a portion of an oligosaccharide that is attached to a protein, lipid, or proteoglycan, for example. Sialyltransferases that are used in the recombinant cells and reaction mixtures of the invention are, in some embodiments, able to transfer sialic acid to the sequence Galβ1,4GlcNAc-, the most common penultimate sequence underlying the terminal sialic acid on fully sialylated carbohydrate structures. Examples of sialyltransferases that can be used in the invention are listed in Table 1.

TABLE 1
Sialyltransferases which use the Galβ1,4GlcNAc saccharide as an acceptor
substrate.

| Sialyltransferase | Source | Structure formed | Ref. |
|---|---|---|---|
| ST6Gal I | Mammalian | NeuAcα2,6Galβ1,4GlcNAc- | 1 |
| ST3Gal III | Mammalian | NeuAcα2,3Galβ1,4GlcNAc- | 1 |
| | | NeuAcα2,3Galβ1,3GlcNAc- | |
| ST3Gal IV | Mammalian | NeuAcα2,3Galβ1,4GlcNAc- | 1 |
| | | NeuAcα2,3Galβ1,3GlcNAc- | |
| ST6Gal II | Photobacterium | NeuAcα2,6Galβ1,4GlcNAc- | 2 |
| ST3Gal V | N. meningitides | NeuAcα2,3Galβ1,4GlcNAc- | 3, 4 |
| | N. gonorrhea | | |
| | C. jejuni | | |
| | (Cst1, Cst2, | | |
| | and Cst3) | | |
| ST 8's | C. jejuni (Cst2) | NeuAcα2,8NeuAcα2,3Galβ1,4GlcNAc- | 4 |

1) Goochee et al. (1991) Bio/Technology 9: 1347-1355
2) Yamamoto et al. (1996) J. Biochem. 120: 104-110
3) Gilbert et al. (1996) J. Biol. Chem. 271: 28271-28276 and U.S. Pat. No. 6,096,529, issued Aug. 1, 2000, which is herein incorporated by reference for all purposes.
4) U.S. Pat. No. 6,503,744, issued Jan. 7, 2003; and U.S. Pat. No. 6,699,705, issued Mar. 2, 2004; each of which is herein incorporated by reference for all purposes. For sialyltransferase nomenclature, see Tsuji et al. (1996) Glycobiology 6: v-xiv).

Conjugation

Depending on the choice of donor substrate or acceptor substrate, the compounds of the invention can be conjugated to another compound. The compounds produced by methods of the invention, in their unconjugated form are generally useful as, e.g., therapeutic agents. The compounds of the invention can be conjugated to a wide variety of compounds to create specific labels, probes, separation media, diagnostic and/or therapeutic reagents, etc. Examples of species to which the compounds of the invention can be conjugated include, for example, biomolecules such as proteins (e.g., antibodies, enzymes, receptors, etc.), nucleic acids (e.g., RNA, DNA, etc.), bioactive molecules (e.g., drugs, toxins, etc.), detectable labels (e.g., fluorophores, radioactive isotopes), solid substrates such as glass or polymeric beads, sheets, fibers, membranes (e.g. nylon, nitrocellulose), slides (e.g. glass, quartz) and probes; etc.

Conjugation can occur when the oligosaccharide products of the invention incorporate a donor substrate or an acceptor substrate that comprises, e.g., a linker moiety, a modifying group, a reactive group, an activated leaving group, a reactive functional group, a reactive ligand, a detectable label such a fluorescent label or a radioactive label, a polymer, a targeting agent, or a cleavable group. In a preferred embodiment, a donor substrate or an acceptor substrate that comprises one or more of the groups listed above is taken up by a host cell. In another preferred embodiment, the donor or acceptor substrate comprises fluoride. In yet another preferred embodiment, the acceptor substrate is glucose-1-F or lactose 1-F. In a further preferred embodiment, a product saccharide comprising fluoride is conjugated to a biomolecule, e.g., a protein, a peptide, a lipid, or a nucleic acid. In still another preferred embodiment, an acceptor substrate or a donor substrate comprises one or more acyl groups.

Linkers

The compounds of the invention can be functionalized with one or more linker moieties, linking the compound to a group, through which the compound may optionally be tethered to another species. The linker can be appended to a glycosyl moiety (e.g., glucose, lactose, or sialic acid), which, in spite of the modification, the serves as a donor substrate or acceptor substrate for an appropriate glycosyltransferase.

Preparation of the Modified Sugar for Use in the Methods of the Present Invention includes attachment of a modifying group to a sugar residue and forming a stable adduct, which is a substrate for a glycosyltransferase. Thus, it is often preferred to use a crosslinking agent to conjugate the modifying group and the sugar. Exemplary bifunctional compounds which can be used for attaching modifying groups to carbohydrate moieties include, but are not limited to, bifunctional poly(ethyleneglycols), polyamides, polyethers, polyesters and the like. General approaches for linking carbohydrates to other molecules are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are treated as benign on the sugar moiety of the nascent modified sugar. The focus of the discussion is for clarity of illustration. Those of skill in the art will appreciate that the discussion is relevant to reactive groups on the modifying group as well.

An exemplary strategy involves incorporation of a protected sulfhydryl onto the sugar using the heterobifunctional crosslinker SPDP (n-succinimidyl-3-(2-pyridyldithio)propionate and then deprotecting the sulfhydryl for formation of a disulfide bond with another sulfhydryl on the modifying group.

If SPDP detrimentally affects the ability of the modified sugar to act as a glycosyltransferase substrate, one of an array of other crosslinkers such as 2-iminothiolane or N-succinimidyl S-acetylthioacetate (SATA) is used to form a disulfide bond. 2-iminothiolane reacts with primary amines, instantly incorporating an unprotected sulfhydryl onto the amine-containing molecule. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategy is exemplary, and not limiting, of linkers of use in the invention. Other crosslinkers are available that can be used in different strategies for crosslinking the modifying group to the peptide. For example, TPCH(S-(2-thiopyridyl)-L-cysteine hydrazide and TPMPH ((S-(2-thiopyridyl) mercapto-propionohydrazide) react with carbohydrate moieties that have been previously oxidized by mild periodate treatment, thus forming a hydrazone bond between the hydrazide portion of the crosslinker and the periodate generated aldehydes. TPCH and TPMPH introduce a 2-pyridylthione protected sulfhydryl group onto the sugar, which can be deprotected with DTT and then subsequently used for conjugation, such as forming disulfide bonds between components.

If disulfide bonding is found unsuitable for producing stable modified sugars, other crosslinkers may be used that incorporate more stable bonds between components. The heterobifunctional crosslinkers GMBS (N-gama-malimidobutyryloxy)succinimide) and SMCC (succinimidyl 4-(N-maleimido-methyl)cyclohexane) react with primary amines, thus introducing a maleimide group onto the component. The maleimide group can subsequently react with sulfhydryls on the other component, which can be introduced by previously mentioned crosslinkers, thus forming a stable thioether bond between the components. If steric hindrance between components interferes with either component's activity or the ability of the modified sugar to act as a glycosyltransferase substrate, crosslinkers can be used which introduce long spacer arms between components and include derivatives of some of the previously mentioned crosslinkers (i.e., SPDP). Thus, there is an abundance of suitable crosslinkers, which are useful; each of which is selected depending on the effects it has on optimal peptide conjugate and modified sugar production.

In another exemplary embodiment, the compound is converted to the corresponding aldehydes or ketone (e.g., by ozonization) and an amine containing carrier molecule is derivatized via reductive amination with the modified compound.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

In an exemplary embodiment, the invention provides a compound according to Formula I, wherein a member selected from a glycosyl residue or Y has the formula:

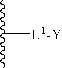

in which L¹ is a member selected from substituted or unsubstituted alkyl, substituted or unsubstituted heteroalkyl and substituted or unsubstituted aryl; and Y is a member selected from protected or unprotected reactive functional groups, detectable labels and targeting moieties.

In another exemplary embodiment, L¹ is an ether or a polyether, preferably a member selected from ethylene glycol, ethylene glycol oligomers and combinations thereof, having a molecular weight of from about 60 daltons to about 10,000 daltons, and more preferably of from about 100 daltons to about 1,000 daltons.

Representative polyether-based substituents include, but are not limited to, the following structures:

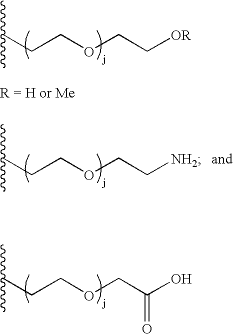

in which is preferably a number from 1 to 100, inclusive. Other functionalized polyethers are known to those of skill in the art, and many are commercially available from, for example, Shearwater Polymers, Inc. (Alabama).

In another preferred embodiment, the linker includes a reactive group for conjugating the oligosaccharide compound to a molecule or a surface. Representative useful reactive groups are discussed in greater detail in the succeeding section. Additional information on useful reactive groups is known to those of skill in the art. See, for example, Hermanson, BIOCONJUGATE TECHNIQUES, Academic Press, San Diego, 1996.

Modified glycosyl donor species (“modified sugars”) are preferably selected from modified sugar nucleotides, activated modified sugars and modified sugars that are simple saccharides or disaccharides that are neither nucleotides nor activated. Any desired carbohydrate structure can be added to a substrate using the methods of the invention. Typically, the structure will be a disaccharide, but the present invention is not limited to the use of modified disaccharide sugars; oligosaccharides and polysaccharides are useful as well.

The modifying group is attached to a sugar moiety by enzymatic means, chemical means or a combination thereof, thereby producing a modified sugar. The sugars are substituted at any position that allows for the attachment of the modifying moiety, yet which still allows the sugar to function as a substrate for the enzyme used to ligate the modified sugar to the substrate or to add a sugar to a modified acceptor saccharide. In preferred embodiments, the modified sugar is an acceptor substrate, e.g., glucose or lactose.

The invention also provides methods for synthesizing a compound using a modified donor substrate sugar or acceptor substrate sugar, e.g., modified-galactose, -glucose, -lactose, -fucose, and -sialic acid. When a modified sialic acid is used, either a sialyltransferase or a trans-sialidase (for α2,3-linked sialic acid only) can be used in these methods.

In some embodiments, an acceptor substrate or a donor substrate comprises one or more acyl groups. Any of the sugars, e.g. glucose, fucose, sialic acid, galactose, GalNAc, GlcNAc, and lactose, comprised in a donor substrate or an acceptor substrate can be acylated with one or more acyl groups on the hydroxyl or amine group of the sugar.

In other embodiments, the modified sugar is comprises a reactive group. Modified sugars that comprises a reactive group, which are useful in the present invention are typically glycosides which have been synthetically altered to include an activated leaving group. As used herein, the term “activated leaving group” refers to those moieties, which are easily displaced in enzyme-regulated nucleophilic substitution reactions. Many activated sugars are known in the art. See, for example, Vocadlo et al., In CARBOHYDRATE CHEMISTRY AND BIOLOGY, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)).

Examples of activating groups include fluoro, chloro, bromo, tosylate ester, mesylate ester, triflate ester and the like. Preferred activated leaving groups, for use in the present invention, are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosatninyl fluoride, α-N-acetylgalactosaminyl fluoride, β-galactosyl fluoride, β-mannosyl fluoride, β-glucosyl fluoride, β-fucosyl fluoride, β-xylosyl fluoride, βsialyl fluoride, β-N-acetylglucosaminyl fluoride and β-N-acetylgalactosaminyl fluoride are most preferred. α-lactosyl fluoride and β-lactosyl fluoride can also be used in the invention.

By way of illustration, glycosyl fluorides can be prepared from the free sugar by first acetylating the sugar and then treating it with HF/pyridine. This generates the thermodynamically most stable anomer of the protected (acetylated) glycosyl fluoride (i.e., the α-glycosyl fluoride). If the less stable anomer (i.e., the β-glycosyl fluoride) is desired, it can be prepared by converting the peracetylated sugar with HBr/HOAc or with HCl to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g. NaOMe/MeOH). In addition, many glycosyl fluorides are commercially available.

Other activated glycosyl derivatives can be prepared using conventional methods known to those of skill in the art. For example, glycosyl mesylates can be prepared by treatment of the fully benzylated hemiacetal form of the sugar with mesyl chloride, followed by catalytic hydrogenation to remove the benzyl groups.

Reactive Functional Groups

As discussed above, certain of the compounds of the invention bear a reactive functional group, such as a component of a linker arm, which can be located at any position on any aryl nucleus or on a chain, such as an alkyl chain, attached to an aryl nucleus, or on the backbone of the chelating agent. These compounds are referred to herein as “reactive ligands.” When the reactive group is attached to an alkyl, or substituted alkyl chain tethered to an aryl nucleus, the reactive group is preferably located at a terminal position of an alkyl chain. Reactive groups and classes of reactions useful in practicing the present invention are generally those that are well known in the art of bioconjugate chemistry. Currently favored classes of reactions available with reactive ligands of the invention are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble the oligosaccharide. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

Detectable Labels

In an exemplary embodiment, the compound prepared by a method of the invention includes a detectable label, such as a fluorophores or radioactive isotope. The detectable label can be appended to a glycosyl moiety (e.g., sialic acid) by means of a linker arm in a manner that still allows the labeled glycosyl moiety serves as a substrate for an appropriate glycosyltransferase as discussed herein.

The embodiment of the invention in which a label is utilized is exemplified by the use of a fluorescent label. Fluorescent labels have the advantage of requiring few precautions in their handling, and being amenable to high-throughput visualization techniques (optical analysis including digitization of the image for analysis in an integrated system comprising a computer). Preferred labels are typically characterized by high sensitivity, high stability, low background, long lifetimes, low environmental sensitivity and high specificity in labeling.

Many fluorescent labels can be incorporated into the compositions of the invention. Many such labels are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

Polymers

In another exemplary embodiment, the invention provides a polymer that includes a subunit according to Formula I. The polymer may be a synthetic polymer (e.g., poly(styrene), poly(acrylamide), poly(lysine), polyethers, polyimines, dendrimers, cyclodextrins, and dextran) or a biopolymer, e.g, polypeptides (e.g., antibody, enzyme, serum protein), saccharide, nucleic acid, antigen, hapten, etc. The polymer may have an activity associated with it (e.g., an antibody) or it may simply serve as a carrier molecule (e.g., a dendrimer).

The carrier molecules may also be used as a backbone for compounds of the invention that are poly- or multi-valent species, including, for example, species such as dimers, trimers, tetramers and higher homologs of the compounds of the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of the invention). Moreover, the framework can be derivatized with a single species of the invention or more than one species of the invention.

Moreover, the properties of the carrier molecule can be selected to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to, for example, replace a hydroxyl group with a diol, or an amine with a quaternary amine, hydroxylamine or similar more water-soluble moiety. In a preferred embodiment, additional water solubility is imparted by substitution at a site not essential for the activity towards the ion channel of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

In another embodiment, the compound produced by the method of the invention is attached to an immunogenic carrier. Commonly used carriers are large molecules that are highly immunogenic and capable of imparting their immunogenicity to a hapten coupled to the carrier. Examples of carriers include, but are not limited to, proteins, lipid bilayers (e.g., liposomes), synthetic or natural polymers (e.g., dextran, agarose, poly-L-lysine) or synthetic organic molecules. Preferred immunogenic carriers are those that are immunogenic, have accessible functional groups for conjugation with a hapten, are reasonably water-soluble after derivitization with a hapten, and are substantially non-toxic in vivo. Presently preferred carriers include, for example protein carriers having a molecular weight of greater than or equal to 5000 daltons, more preferably, albumin or hemocyanin.

The immunogenicity of compositions prepared by the methods of the present invention may further be enhanced by linking the composition to one or more peptide sequences that are able to a elicit a cellular immune response (see, e.g., WO 94/20127). Peptides that stimulate cytotoxic T lymphocyte (CTL) responses as well as peptides that stimulate helper T lymphocyte (HTL) responses are useful for linkage to the compounds of the invention. The peptides can be linked by a linker moiety as discussed above. An exemplary linker is typically comprised of relatively small, neutral molecules, such as amino acids or amino acid mimetics, which are uncharged under physiological conditions.

A compound prepared by a method of the invention may be linked to a T helper peptide that is recognized by T helper cells in the majority of the population. This can be accomplished by selecting amino acid sequences that bind to many, most, or all of the HLA class II molecules. An example of such a T helper peptide is tetanus toxoid at positions 830-843 (see, e.g., Panina-Bordignon et al., Eur. J. Immunol. 19: 2237-2242 (1989)).

Further, a compound prepared by a method of the invention may be linked to multiple antigenic determinants to enhance immunogenicity. For example, in order to elicit recognition by T cells of multiple HLA types, a synthetic peptide encoding multiple overlapping T cell antigenic determinants (cluster peptides) may be used to enhance immunogenicity (see, e.g., Ahlers et al., J. Immunol. 150: 5647-5665 (1993)). Such cluster peptides contain overlapping, but distinct antigenic determinants. The cluster peptide may be synthesized colinearly with a peptide of the invention. The cluster peptide may be linked to a compound of the invention by one or more spacer molecules.

A peptide composition comprising a compound of the invention linked to a cluster peptide may also be used in conjunction with a cluster peptide linked to a CTL-inducting epitope. Such compositions may be administered via alternate routes or using different adjuvants.

Alternatively multiple peptides encoding CTL and/or HTL epitopes may be used in conjunction with a compound of the invention.

Many methods are known to those of skill in the art for coupling a hapten to a carrier. In an exemplary embodiment, a disaccharide or oligosaccharide prepared by the method of the invention includes a sulfhydryl group that is readily combined with keyhole limpet hemocyanin, which has been activated by SMCC (succinimidyl-4-(N-maleimidomethyl)cyclohexane-1-carboxylate), Dewey et al., Proc. Natl. Acad. Sci. USA 84: 5374-5378 (1987). The sulfhydryl-bearing carbohydrate useful in this method can be synthesized by a number of art-recognized methods. For example, a carbohydrate bearing a terminal carboxyl group is coupled with cysteamine, using a dehydrating agent, such as dicyclohexylcarbodiimide (DCC), to form a dimeric glycolipid, linked via a disulfide bridge. The disulfide bridge is cleaved by reduction, affording the monomeric sulfhydryl-derivatized product.

In yet another preferred embodiment, the composition includes a linker moiety situated between the product and the carrier. The discussion above regarding the characteristics of linker moieties is substantially applicable to the present embodiment. In an exemplary embodiment, the linker arm includes a poly(ethyleneglycol) (PEG) group. Bifunctional PEG derivative appropriate for use in this method are commercially available (Shearwater Polymers) or can be prepared by methods well known in the art. In an exemplary embodiment, the SMCC activated KLH, infra, is reacted with a PEG-disaccharide or oligosaccharide conjugate, bearing a sulfhydryl group. An appropriate conjugate can be prepared by a number of synthetic routes accessible to those of skill in the art. For example, a commercially available product, such as t-Boc-NH-PEG-NH₂, is reacted with a carboxyl terminal disaccharide or oligosaccharide in the presence of a dehydrating agent (e.g., DCC), thereby forming the PEG amide of the disaccharide or oligosaccharide. The t-Boc group is removed by acid treatment (e.g., trifluoroacetic acid, TFA), to afford the deprotected amino PEG amide of the disaccharide or oligosaccharide. The deprotected disaccharide or oligosaccharide is subsequently reacted with a sulfhydryl protected molecule, such as 3-mercaptopropionic acid or a commercially available thiol and amine protected cysteine, in the presence of a dehydrating agent. The thiol group is then deprotected and the conjugate is reacted with the SMCC activated KLH to provide an autoinducer analogue linked to a carrier via a PEG spacer group.

The exemplary embodiments presented above are intended to illustrate general reaction schemes that are useful in preparing certain of the compounds of the present invention and should not be interpreted as limiting the scope of the invention or the pathways useful to produce the compounds of the invention.

Targeting Moieties

In addition to providing a polymeric “support” or backbone for TIAM and other cheating agents, carrier molecules can be used to target ligands (or complexes) of the invention to a specific region within the body or tissue, or to a selected species or structure in vitro. Selective targeting of an agent by its attachment to a species with an affinity for the targeted region is well known in the art. Both small molecule and polymeric targeting agents are of use in the present invention.

The ligands (or complexes) can be linked to targeting agents that selectively deliver it to a cell, organ or region of the body. Exemplary targeting agents such as antibodies, ligands for receptors, lectins, saccharides, antibodies, and the like are recognized in the art and are useful without limitation in practicing the present invention. Other targeting agents include a class of compounds that do not include specific molecular recognition motifs include macromolecules such as poly(ethylene glycol), polysaccharide, polyamino acids and the like, which add molecular mass to the ligand. The ligand-targeting agent conjugates of the invention are exemplified by the use of a nucleic acid-ligand conjugate. The focus on ligand-oligonucleotide conjugates is for clarity of illustration and is not limiting of the scope of targeting agents to which the ligands (or complexes) of the invention can be conjugated. Moreover, it is understood that “ligand” refers to both the free ligand and its metal complexes.

Exemplary nucleic acid targeting agents include aptamers, antisense compounds, and nucleic acids that form triple helices. Typically, a hydroxyl group of a sugar residue, an amino group from a base residue, or a phosphate oxygen of the nucleotide is utilized as the needed chemical functionality to couple the nucleotide-based targeting agent to the ligand. However, one of skill in the art will readily appreciate that other “non-natural” reactive functionalities can be appended to a nucleic acid by conventional techniques. For example, the hydroxyl group of the sugar residue can be converted to a mercapto or amino group using techniques well known in the art.

Aptamers (or nucleic acid antibody) are single- or double-stranded DNA or single-stranded RNA molecules that bind specific molecular targets. Generally, aptamers function by inhibiting the actions of the molecular target, e.g., proteins, by binding to the pool of the target circulating in the blood. Aptamers possess chemical functionality and thus, can covalently bond to ligands, as described herein.

Although a wide variety of molecular targets are capable of forming non-covalent but specific associations with aptamers, including small molecules drugs, metabolites, cofactors, toxins, saccharide-based drugs, nucleotide-based drugs, glycoproteins, and the like, generally the molecular target will comprise a protein or peptide, including serum proteins, kinins, eicosanoids, cell surface molecules, and the like. Examples of aptamers include Gilead's antithrombin inhibitor GS 522 and its derivatives (Gilead Science, Foster City, Calif.). See also, Macaya et al. Proc. Natl. Acad. Sci. USA 90:3745-9 (1993); Bock et al. Nature (London) 355:564-566 (1992) and Wang et al. Biochem. 32:1899-904 (1993).

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

Cleaveable Groups

The invention also provides methods of preparing oligosaccharide conjugates that are linked to another moiety (e.g., polymer, targeting moiety, detectable label, solid support) via a linkage that is designed to cleave, releasing the saccharide conjugate. Cleaveable groups include bonds that are reversible (e.g., easily hydrolyzed) or partially reversible (e.g., partially or slowly hydrolyzed). Cleavage of the bond can occur through biological or physiological processes. In other embodiments, the physiological processes cleave bonds at other locations within the complex (e.g., removing an ester group or other protecting group that is coupled to an otherwise sensitive chemical functionality) before cleaving the bond between the agent and dendrimer, resulting in partially degraded complexes. Other cleavages can also occur, for example, between a spacer and targeting agent and the spacer and the ligand.

In an exemplary embodiment, the linkage used in the method of the invention is degraded by enzymes such as non-specific aminopeptidases and esterases, dipeptidyl carboxypeptidases, proteases of the blood clotting cascade, and the like.

Alternatively, cleavage is through a nonenzymatic process. For example, chemical hydrolysis may be initiated by differences in pH experienced by the complex. In such a case, the complex may be characterized by a high degree of chemical lability at physiological pH of 7.4, while exhibiting higher stability at an acidic or basic pH in the delivery vehicle. An exemplary complex, which is cleaved in such a process is a complex incorporating a N-Mannich base linkage within its framework.

Another exemplary group of cleaveable compounds are those based on non-covalent protein binding groups discussed herein.

The susceptibility of the cleaveable group to degradation can be ascertained through studies of the hydrolytic or enzymatic conversion of the group. Generally, good correlation between in vitro and in vivo activity is found using this method. See, e.g., Phipps et al., J. Pharm. Sciences 78:365 (1989). The rates of conversion are readily determined, for example, by spectrophotometric methods or by gas-liquid or high-pressure liquid chromatography. Half-lives and other kinetic parameters may then be calculated using standard techniques. See, e.g., Lowry et al. MECHANISM AND THEORY IN ORGANIC CHEMISTRY, 2nd Ed., Harper & Row, Publishers, New York (1981).

### IV. Enzymatic Systems for Synthesizing Oligosaccharides

The invention provides cells and methods of using the cells to produce oligosaccharides. The cells typically express a glycosyltransferase while growing on a defined growth medium and also produce a donor substrate molecule from precursors in the growth medium. The glycosyltransferase catalyzes the transfer of a sugar moiety from the donor substrate to an acceptor saccharide. In some embodiments, the glycosyltransferase is encoded by a heterologous nucleic acid (i.e., a nucleic acid that is not native to the cell, or that is modified from its native form in the cell; such sialyltransferases are referred to herein as “recombinant,” “exogenous,” or “heterologous” glycosyltransferases). The cells can also contain one or more genes that encode enzymes involved in the synthesis of the sugar moiety and/or an enzyme that catalyzes formation of the donor substrate, e.g., an activated sugar molecule, such as CMP-sialic acid, GDP-fucose, UDP-glucose or UDP-galactose. In some embodiments, at least one of the enzymes involved in the synthesis of the donor substrate molecule, i.e., an enzymatic system for forming a donor substrate, is encoded by a heterologous nucleic acid (i.e., as above a nucleic acid that is not native to the cell, or that is modified from its native form in the cell; such enzymes are referred to herein as “recombinant,” “exogenous,” or “heterologous” enzymes, proteins, or polypeptides). The enzymes are typically part of an enzymatic system for producing the sugar or for producing an activated sugar molecule. The heterologous nucleic acids can be, for example, polynucleotides that are not endogenous to the cell, or can be a modified form of a polynucleotide that is endogenous to the cell. In some applications, the cells will contain more than one heterologous glycosyltransferase gene and/or more than one heterologous gene that encodes enzymes involved in the synthesis of the activated sugar molecule. In other embodiments, the cells will also include a sialyltransferase and a second glycosyltransferase

A. Synthesis of Donor Substrates and Precursors of Donor Substrates

Glycosyltransferase reactions require a nucleotide sugar which serves as sugar donor. Enzymes that are involved in synthesis of a nucleotide sugar or synthesis of the sugar are also called accessory enzymes. Accessory enzymes include those enzymes that are involved in the formation of a nucleotide sugar. The accessory enzyme can be involved in attaching the sugar to a nucleotide, or can be involved in making the sugar or the nucleotide, for example. Because the organism continues to produce either the nucleotide or sugar nucleotide and the recombinant enzymes are also present, the continuous production of product can occur starting from low cost raw materials. Recycling of the spent nucleotide produced from the transfer of the sugar from the sugar nucleotide during product formation can also occur as the organism contains the enzymatic processes to reform either the sugar nucleotide or nucleotide. Accessory enzymes that are involved in synthesis of nucleotide sugars are well known to those of skill in the art. For a review of bacterial polysaccharide synthesis and gene nomenclature, see, e.g., Reeves et al., Trends Microbiol. 4: 495-503 (1996).

Generally, the recombinant cells of the invention add one or more sugar moieties to the acceptor saccharide using one or more glycosyltransferases, either exogenous or endogenous. In some embodiments, the recombinant cells of the invention can naturally produce the sugar nucleotide(s) that serves as sugar donor(s) for the glycosyltransferase(s) produced by the cell, as well as the nucleotide to which the sugar molecule is attached. However, some cells do not naturally produce sufficient amounts of either or both of the nucleotide or the nucleotide sugar to produce the desired quantities of product saccharide. In such situations, the recombinant cells of the invention contain at least one heterologous gene that encodes an accessory enzyme for making one or more nucleotide sugars.

For example, sialyltransferases require a CMP-sialic acid molecule, i.e., an activated sialic acid molecule, to serve as a donor of a sialic acid moiety. In some embodiments, the recombinant cells of the invention can naturally produce the CMP-sialic acid molecule that serves as a sugar donor for the sialyltransferase produced by the cell, as well as the nucleotide to which the sialic acid moiety is attached. However, some cells do not naturally produce sufficient amounts of either or both of the CMP or the sialic acid to produce the desired quantities of product saccharide In such situations, the recombinant cells of the invention can contain at least one heterologous gene that encodes an accessory enzyme, involved in synthesis of CMP-sialic acid.

The enzymatic system for forming the nucleotide sugar includes, in presently preferred embodiments, an enzyme encoded by a heterologous gene. Such cells provide a means for forming a desired nucleotide sugar that is not normally produced by the wild-type cell, or is not produced at a sufficiently high level by the wild-type cell. In some instances, the enzyme encoded by the heterologous gene can convert a nucleotide or nucleotide sugar that is produced by the cell into a different nucleotide sugar that can serve as a substrate for the desired coupling reaction. In other cases, the enzyme encoded by the heterologous gene can synthesize a nucleotide sugar from other substrates (e.g., nucleotides) that are found in the cell, either endogenously or as a result of the substrate having been added to the cell. Multiple nucleotide sugar synthesis and/or conversion reactions can be achieved by using a cell that contains more than one heterologous gene that encodes an enzyme involved in nucleotide sugar synthesis.

The genes encoding enzymes for an entire sugar nucleotide regeneration cycle can be introduced into an organism along with the glycosyltransferase of interest. The resulting recombinant cells can thus produce both the desired nucleotide sugar and the desired product. Pathways and enzymes that are involved in synthesis of nucleotide sugars are well known to those of skill in the art. For a review of bacterial polysaccharide synthesis and gene nomenclature, see, e.g., Reeves et al. (1996) Trends Microbiol. 4: 495-503. Examples of cycle enzymes that are of use in producing various nucleotide sugars are listed in Table 2.

TABLE 2
Cycle Enzymes¹

GlcNAc Cycle
UDP-GlcNAc Pyrophosphorylase
GlcNAc/GalNAc Kinase
GlcNAc Transferase
Gal Cycle-1
Gal Kinase
UDP-Gal Pyrophosphorylase
Gal Transferase
Gal Cycle-2
UDP-Gal 4′-Epimerase
UDP-Glc Pyrophosphorylase
Hexokinase Kinase
Phosphoglucomutase
ST Cycle
ST fusion (sialyltransferase fused CMP-SA
synthetase)*
*(or sialyltransferase and CMP-SA synthetase)
NeuAc Aldolase
GlcNAc Epimerase
Fuc Cycle-1
GDP-Fuc Epimerase/reductase
GDP-Fuc Dehydratase
GDP-Man Pyrophosphorylase
Hexokinase
Phosphomannomutase
Fucosyl Transferase
GalNAc Cycle-1
UDP-GalNAc Epimerase
UDP-GlcNAc Pyrophosphorylase
GlcNAc 1-Phospho Kinase*
*(or Hexokinase and GlcNAc
Phosphomutase)
GlcNAc Transferase
GalNAc Cycle-2
UDP-GalNAc Pyrophosphorylase
GlcNAc Transferase
GlcNAc/GalNAc kinase
Man Cycle
GDP-Man Pyrophosphorylase
Hexokinase
Phosphomannomutase
Man Transferase
Fuc Cycle-2
GDP-Fuc Pyrophosphorylase
Fucose 1-phosphokinase
Fucosyl Transferase
¹Each of the cycle processes listed below requires either a nucleotide triphosphate source or the enzymes required to regenerate the nucleotide to its nucleotide triphosphate form.

By introducing a nucleic acid that encodes an accessory enzyme into a cell that contains a substrate for the accessory enzyme, or that takes up a substrate of the accessory enzyme from the medium, one can modify one or more pathways that are involved in nucleotide sugar production. Methods to identify and obtain nucleic acids that encode accessory enzymes are known to those of skill. The methods described below for obtaining glycosyltransferase-encoding nucleic acids are also applicable to obtaining nucleic acids that encode enzymes involved in the formation of nucleotide sugars. For example, one can use directly a nucleic acid known in the art, some of which are listed herein, or one can use the known nucleic acid as a probe to isolate corresponding nucleic acids from other organisms of interest. The isolation of polynucleotides that encode nucleotide sugar synthetic enzymes can be performed by a number of techniques well known to those skilled in the art. For instance, oligonucleotide probes that selectively hybridize to a particular gene described herein can be used to identify the desired gene in DNA isolated from another organism. The use of such hybridization techniques for identifying homologous genes is well known in the art are otherwise as described above.

1. CMP-Sialic Acid Regeneration

To obtain recombinant cells of the invention that are useful for sialylation reactions, one can introduce a gene that encodes a CMP-sialic acid synthetase (EC 2.7.7.43, CMP-N-acetylneuraminic acid synthetase). Such genes are available from, for example, Mus musculus (GenBank AJ006215, Munster et al., Proc. Natl. Acad. Sci. U.S.A. 95: 9140-9145 (1998)), rat (Rodríguez-Aparicio et al. (1992) J. Biol. Chem. 267: 9257-63), Haemophilus ducreyi (Tullius et al. (1996) J. Biol. Chem. 271: 15373-80), Neisseria meningitidis (Ganguli et al. (1994) J. Bacteriol. 176: 4583-9), group B streptococci (Haft et al. (1994) J. Bacteriol. 176: 7372-4), and E. coli (GenBank J05023, Zapata et al. (1989) J. Biol. Chem. 264: 14769-14774). CMP-sialic acid synthetase polypeptides and their encoding nucleic acids have also been identified in Campylobacter, Pasteurella, and Pseudomonas.

In a preferred embodiment for making a sialylated product saccharide, an enzymatic system for synthesizing sialic acid from N-acetylglucosamine (GlcNac) is used. An enzymatic system for synthesizing sialic acid from GlcNac refers to an enzymatic system that converts a precursor of sialic acid to sialic acid. Generally, the precursor of sialic acid is provided in a growth or culture medium and the enzymatic system for synthesizing sialic acid is expressed by the microorganisms of the invention. In some embodiments, the enzymatic system for synthesizing sialic acid from GlcNac is used. The GlcNAc is provided in the culture medium and appropriate enzymes are selected for the conversion of GlcNAc to sialic acid based, at least in part, on the endogenous enzymes of the host cell. Those of skill are aware that more than one pathway exists to convert GlcNAc to sialic acid and that a variety of enzymes can be combined to perform the conversion. For example, in Neisseria, GlcNAc is converted to sialic acid through the actions of at least two enzymes, a GlcNAc epimerase (the SiaA protein, Accession Number M95053 region: 174.1307) and an N-acetyl neuraminic acid (NANA) condensing polypeptide (the SiaC protein, Accession Number M95053 region: 1998.3047). The SiaC protein condenses N-acetyl-D-mannosamine and pyruvate to form NANA. In E. coli K12, for example, UDP-GlcNAc is converted to N-acetyl-D-mannosamine (ManNAc) by UDP-GlcNAc epimerase (the NeuC protein, Accession number M84026). The NeuB gene product (a sialate synthase protein, Accession number AAC43302, encoded by Accession number U05248, region 723-1763) condenses ManNAc and phosphoenol pyruvate to form NANA, which is converted to CMP-NANA by the NeuA gene product (a CMP-sialate synthase protein, Accession number J05023). See, e.g., Ringenberg et al., Glycobiology 11:533-539 (2001). While specific enzymes are listed, those of skill will recognize that other enzymes from different organisms can be used in an enzymatic system for synthesizing sialic acid from GlcNac. In many organisms, sialic acid synthesis proteins are encoded by nucleic acids at localized regions of the chromosomes, e.g., operons. While specific enzymes are listed, those of skill will recognize that homologues of the above enzymes isolated from different organisms can be used in an enzymatic system for synthesizing sialic acid from GlcNAc. Individual nucleic acids that encode sialic acid synthetic enzymes can be included in an expressin cassette, as can an operon the encodes all or a portion of a sialic acid synthetic pathway.

2. UDP-Gal Regeneration

An illustrative example of a recombinant cell that is useful for producing a galactosylated product saccharide contains a heterologous galactosyltransferase gene. However, galactosyltransferases generally use as a galactose donor the activated nucleotide sugar UDP-Gal, which is comparatively expensive. To reduce the expense of the reaction, one can introduce into the cell (or increase the level of expression of) one or more genes that encode enzymes that are involved in the biosynthetic pathway which leads to UDP-Gal or conversely, one can inactivate an enzyme that catalyzes the conversion of UDP-Gal to a molecule that is not a nucleotide sugar.

For example, glucokinase (EC 2.7.1.12) catalyzes the phosphorylation of glucose to form Glc-6-P. Genes that encode glucokinase have been characterized (e.g., E. coli: GenBank AE000497 U00096, Blattner et al. (1997) Science 277: 1453-1474; Bacillus subtilis: GenBank Z99124, AL009126, Kunst et al. (1997) Nature 390, 249-256), and thus can be readily obtained from many organisms by, for example, hybridization or amplification. A recombinant cell that contains this gene, as well as the subsequent enzymes in the pathway as set forth below, will thus be able to form UDP-glucose from readily available glucose, which can be either produced by the organism or added to the reaction mixture.

The next step in the pathway leading to UDP-Gal is catalyzed by phosphoglucomutase (EC 5.4.2.2), which converts Glc-6-P to Glc-1-P. Again, genes encoding this enzyme have been characterized for a wide range of organisms (e.g., Agrobacterium tumefaciens: GenBank AF033856, Uttaro et al. Gene 150: 117-122 (1994) [published erratum appears in Gene (1995) 155:141-3]; Entamoeba histolytica: GenBank Y14444, Ortner et al., Mol. Biochem. Parasitol. 90, 121-129 (1997); Mesembryanthemum crystallinum: GenBank U84888; S. cerevisiae: GenBank X72016, U09499, X74823, Boles et al., Eur. J. Biochem. 220: 83-96 (1994), Fu et al., J. Bacteriol. 177 (11), 3087-3094 (1995); human: GenBank M83088 (PGM1), Whitehouse et al., Proc. Nat'l. Acad. Sci. U.S.A. 89: 411-415 (1992), Xanthomonas campestris: GenBank M83231, Koeplin et al., J. Bacteriol. 174: 191-199 (1992); Acetobacter xylinum: GenBank L24077, Brautaset et al., Microbiology 140 (Pt 5), 1183-1188 (1994); Neisseria meningitidis: GenBank U02490, Zhou et al., J. Biol. Chem. 269 (15), 11162-11169 (1994).

UDP-glucose pyrophosphorylase (EC 2.7.7.9) catalyzes the next step in the pathway, conversion of Glc-1-P to UDP-Glc. Genes encoding UDP-Glc pyrophosphorylase are described for many organisms (e.g., E. coli: GenBank M98830, Weissborn et al., J. Bacteriol. 176: 2611-2618 (1994); Cricetulus griseus: GenBank AF004368, Flores-Diaz et al., J. Biol. Chem. 272: 23784-23791 (1997); Acetobacter xylinum: GenBank M76548, Brede et al., J. Bacteriol. 173, 7042-7045 (1991); Pseudomonas aeruginosa (galU): GenBank AJ010734, U03751; Streptococcus pneumoniae: GenBank AJ004869; Bacillus subtilis: GenBank Z22516, L12272; Soldo et al., J. Gen. Microbiol. 139 (Pt 12), 3185-3195 (1993); Solanum tuberosum: GenBank U20345, L77092, L77094, L77095, L77096, L77098, U59182, Katsube et al., J. Biochem. 108: 321-326 (1990); Hordeum vulgare (barley): GenBank X91347; Shigella flexneri: GenBank L32811, Sandlin et al., Infect. Immun. 63: 229-237 (1995); human: GenBank U27460, Duggleby et al., Eur. J. Biochem. 235 (1-2), 173-179 (1996); bovine: GenBank L14019, Konishi et al., J. Biochem. 114, 61-68 (1993).

Finally, UDP-Glc 4′-epimerase (UDP-Gal 4′-epimerase; EC 5.1.3.2) catalyzes the conversion of UDP-Glc to UDP-Gal. The Streptococcus thermophilus UDPgalactose 4′-epimerase gene described by Poolman et al. (J. Bacteriol 172: 4037-4047 (1990)) is a particular example of a gene that is useful in the present invention. UDPglucose 4′-epimerase-encoding polynucleotides of other organisms can be used in the present invention, so long as polynucleotides are under the control of expression control sequences that function in E. coli or other desired host cell. Exemplary organisms that have genes encoding UDPglucose 4-epimerase include E. coli, K. pneumoniae, S. lividans, and E. stewartii, as well as Salmonella and Streptococcus species. Nucleotide sequences are known for UDP-Glc 4′-epimerases from several organisms, including Pasteurella haemolytica, GenBank U39043, Potter et al., Infect. Immun. 64 (3), 855-860 (1996); Yersinia enterocolitica, GenBank Z47767, X63827, Skurnik et al., Mol. Microbiol. 17: 575-594 (1995); Cyamopsis tetragonoloba: GenBank AJ005082; Pachysolen tannophilus: GenBank X68593, Skrzypek et al., Gene 140 (1), 127-129 (1994); Azospirillum brasilense: GenBank Z25478, De Troch et al., Gene 144 (1), 143-144 (1994); Arabidopsis thaliana: GenBank Z54214, Dormann et al., Arch. Biochem. Biophys. 327: 27-34 (1996); Bacillus subtilis: GenBank X99339, Schrogel et al., FEMS Microbiol. Lett. 145: 341-348 (1996); Rhizobium meliloti: GenBank X58126 S81948, Buendia et al., Mol. Biol. 5: 1519-1530 (1991); Rhizobium leguminosarum: GenBank X96507; Erwinia amylovora: GenBank X76172, Metzger et al., J. Bacteriol. 176: 450-459 (1994); S. cerevisiae: GenBank X81324 (cluster of epimerase and UDP-glucose pyrophosphorylase), Schaaff-Gerstenschlager, Yeast 11: 79-83 (1995); Neisseria meningitidis: GenBank U19895, L20495, Lee et al., Infect. Immun. 63: 2508-2515 (1995), Jennings et al., Mol. Microbiol. 10: 361-369 (1993); and Pisum sativum: GenBank U31544.

Often, genes encoding enzymes that make up a pathway involved in synthesizing nucleotide sugars are found in a single operon or region of chromosomal DNA. For example, the Xanthomonas campestris phosphoglucomutase, phosphomannomutase, (xanA), phosphomannose isomerase, and GDP-mannose pyrophosphorylase (xanB) genes are found on a single contiguous nucleic acid fragment (Koeplin et al., J. Bacteriol. 174, 191-199 (1992)). Klebsiella pneumoniae galactokinase, galactose-1-phosphate uridyltransferase, and UDP-galactose 4′-epimerase are also found in a single operon (Peng et al. (1992) J. Biochem. 112: 604-608). Many other examples are described in the references cited herein.

An alternative way to construct a cell that makes UDP-Gal is to introduce into the cell genes that encode enzymes involved in UDP-Gal synthesis. This pathway begins with UDP-Gal pyrophosphorylase (galactose-1-phosphate uridyltransferase), which converts Gal-1-P to UDP-Gal. Genes that encode UDP-Gal pyrophosphorylase have been characterized for several organisms, including, for example, Rattus norvegicus: GenBank L05541, Heidenreich et al., DNA Seq. 3: 311-318 (1993); Lactobacillus casei: GenBank AF005933 (cluster of galactokinase (galK), UDP-galactose 4′-epimerase (galE), galactose 1-phosphate-uridyltransferase (galT)), Bettenbrock et al., Appl. Environ. Microbiol. 64: 2013-2019 (1998); E. coli: GenBank X06226 (galE and galT for UDP-galactose-4-epimerase and galactose-1-P uridyltransferase), Lemaire et al., Nucleic Acids Res. 14: 7705-7711 (1986)); B. subtilis: GenBank Z99123 AL009126; Neisseria gonorrhoeae: GenBank Z50023, Ullrich et al., J. Bacteriol. 177: 6902-6909 (1995); Haemophilus influenzae: GenBank X65934 (cluster of galactose-1-phosphate uridyltransferase, galactokinase, mutarotase and galactose repressor), Maskell et al., Mol. Microbiol. 6: 3051-3063 (1992), GenBank M12348 and M12999, Tajima et al., Yeast 1: 67-77 (1985)); S. cerevisiae: GenBank X81324, Schaaff-Gerstenschlager et al., Yeast 11: 79-83 (1995); Mus musculus: GenBank U41282; human: GenBank M96264, M18731, Leslie et al., Genomics 14: 474-480 (1992), Reichardt et al., Mol. Biol. Med. 5: 107-122 (1988); Streptomyces lividans: M18953 (galactose 1-phosphate uridyltransferase, UDP-galactose 4-epimerase, and galactokinase), Adams et al., J. Bacteriol. 170: 203-212 (1988).

UDP-GlcNAc 4′ epimerase (UDP-GalNAc 4′-epimerase) (EC 5.1.3.7), which catalyzes the conversion of UDP-GlcNAc to UDP-GalNAc, and the reverse reaction, is also suitable for use in the recombinant cells of the invention. Several loci that encode this enzyme are described above. See also, U.S. Pat. No. 5,516,665.

3. GDP-Fucose Regeneration

Another example of a recombinant cell provided by the invention is used for producing a fucosylated product saccharide. The donor nucleotide sugar for fucosyltransferases is GDP-fucose, which is relatively expensive to produce. In some embodiments of the invention, the cost of obtaining GDP-fucose is reduced by introducing into the recombinant cell one or more exogenous genes that encode enzymes that catalyze a GDP-fucose cycle.

To reduce the cost of producing the fucosylated oligosaccharide, the invention provides cells that can convert the relatively inexpensive GDP-mannose into GDP-fucose. These cells contain at least one exogenous gene that encodes a GDP-mannose dehydratase, a GDP-4-keto-6-deoxy-D-mannose 3,5-epimerase, or a GDP-4-keto-6-deoxy-L-glucose 4-reductase. Cells that contain each of these enzyme activities can convert GDP-mannose into GDP-fucose. The introduction of a fucosyltransferase into the cell results in a cell that can fucosylate an oligosaccharide acceptor using GDP-mannose, rather than GDP-fucose, as the starting material.

The nucleotide sequence of an E. coli gene cluster that encodes GDP-fucose-synthesizing enzymes is described by Stevenson et al. (1996) J. Bacteriol. 178: 4885-4893; GenBank Accession No. U38473). This gene cluster had been reported to include an open reading frame for GDP-mannose dehydratase (nucleotides 8633-9754; Stevenson et al., supra.). It was recently discovered that this gene cluster also contains an open reading frame that encodes an enzyme that has both 3′, 5′ epimerization and 4′-reductase activities (see, PCT Patent Application No. US99/00893, which was published as WO99/36555 on Jul. 22, 1999), and thus is capable of converting the product of the GDP-mannose dehydratase reaction (GDP-4-keto-6-deoxymannose) to GDP-fucose. This ORF, which is designated YEF B, is found between nucleotides 9757-10722. Prior to this discovery that YEF B encodes an enzyme having two activities, it was not known whether one or two enzymes were required for conversion of GDP-4-keto-6-deoxymannose to GDP-fucose. The nucleotide sequence of a gene encoding the human Fx enzyme is found in GenBank Accession No. U58766.

The recombinant cells can also include a gene that encodes GDP-Man pyrophosphorylase (EC 2.7.7.22), which converts Man-1-P to GDP-Man. When present along with an enzyme such as those described above which catalyze the conversion of GDP-Man to GDP-Fuc, such cells can synthesize GDP-Fuc starting from the relatively inexpensive Man-1-P. Suitable genes are known from many organisms, including E. coli: GenBank U13629, AB010294, D43637 D13231, Bastin et al., Gene 164: 17-23 (1995), Sugiyama et al., J. Bacteriol. 180: 2775-2778 (1998), Sugiyama et al., Microbiology 140 (Pt 1): 59-71 (1994), Kido et al., J. Bacteriol. 177: 2178-2187 (1995); Klebsiella pneumoniae: GenBank AB010296, AB010295, Sugiyama et al., J. Bacteriol. 180: 2775-2778 (1998); Salmonella enterica: GenBank X56793 M29713, Stevenson et al., J. Bacteriol. 178: 4885-4893 (1996).

The cells of the invention for fucosylating a saccharide acceptor can also utilize enzymes that provide a minor or “scavenge” pathway for GDP-fucose formation. In this pathway, free fucose is phosphorylated by fucokinase to form fucose 1-phosphate, which, along with guanosine 5′-triphosphate (GTP), is used by GDP-fucose pyrophosphorylase to form GDP-fucose (Ginsburg et al., J. Biol. Chem., 236: 2389-2393 (1961) and Reitman, J. Biol. Chem., 255: 9900-9906 (1980)). GDP-fucose pyrophosphorylase-encoding nucleic acids are described in copending, commonly assigned U.S. patent application Ser. No. 08/826,964, filed Apr. 9, 1997. Fucokinase-encoding nucleic acids are described for, e.g., Haemophilus influenzae (Fleischmann et al. (1995) Science 269:496-512) and E. coli (Lu and Lin (1989) Nucleic Acids Res. 17: 4883-4884).

4. Other Accessory Enzymes

Other pyrophosphorylases are known that convert a sugar phosphate into a nucleotide sugar. For example, UDP-GalNAc pyrophosphorylase catalyzes the conversion of GalNAc to UDP-GalNAc. UDP-GlcNAc pyrophosphorylase (EC 2.7.7.23) converts GlcNAc-1-P to UDP-GlcNAc (B. subtilis: GenBank Z99104 AL009126, Kunst et al., supra.; Candida albicans: GenBank AB011003, Mio et al., J. Biol. Chem. 273 (23), 14392-14397 (1998); Saccharomyces cerevisiae: GenBank AB011272, Mio et al., supra.; human: GenBank AB011004, Mio et al., supra.).

B. Synthesis of Oligosaccharides Using Glycosyltransferases

For enzymatic saccharide syntheses of oligosaccharides, the recombinant cells of the invention contain at least one heterologous gene that encodes a glycosyltransferase. Many glycosyltransferases are known, as are their polynucleotide sequences. See, e.g., “The WWW Guide To Cloned Glycosyltransferases,” (www.vei.co.uk/TGN/gt_guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed in the cells of the invention include, but are not limited to, sialyltransferases, galactosyltransferases, fucosyltransferases, glucosyltransferases, N-acetylgalactosaminyltransferases, N-acetylglucosaminyltransferases, glucuronyltransferases, mannosyltransferases, glucuronic acid transferases, galacturonic acid transferases, and oligosaccharyltransferases. Suitable glycosyltransferases include those obtained from eukaryotes, as well as from prokaryotes.

For example, many mammalian glycosyltransferases have been cloned and expressed and the recombinant proteins have been characterized in terms of donor and acceptor specificity. The glycosyltransferases have also been investigated through site directed mutagenesis in attempts to define residues involved in either donor or acceptor specificity, thus facilitating the identification of catalytic domains that are useful in making recombinant cells that express fusion proteins as discussed herein (Aoki et al. (1990) EMBO. J. 9: 3171-3178; Harduin-Lepers et al. (1995) Glycobiology 5(8): 741-758; Natsuka and Lowe (1994) Current Opinion in Structural Biology 4: 683-691; Zu et al. (1995) Biochem. Biophys. Res. Comm. 206(1): 362-369; Seto et al. (1995) Eur. J. Biochem. 234: 323-328; Seto et al. (1997) J. Biol. Chem. 272: 14133-141388).

Glycosyltransferase nucleic acids and methods of obtaining such nucleic acids, are known to those of skill in the art. Glycosyltransferase nucleic acids (e.g., cDNA, genomic, or subsequences probes)) can be cloned, or amplified by in vitro methods such as the polymerase chain reaction (PCR), the ligase chain reaction (LCR), the transcription-based amplification system (TAS), or the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al. (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Nat'l. Acad. Sci. USA 86: 1173; Guatelli et al. (1990) Proc. Nat'l. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

DNA that encodes glycosyltransferase proteins or subsequences, including sialyltransferases, as well as DNA that encodes the enzymes involved in formation of nucleotide sugars described above, can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. In one preferred embodiment, a nucleic acid encoding a glycosyltransferase can be isolated by routine cloning methods. A nucleotide sequence of a glycosyltransferase as provided in, for example, GenBank or other sequence database can be used to provide probes that specifically hybridize to a glycosyltransferase gene in a genomic DNA sample, or to a glycosyltransferase mRNA in a total RNA sample (e.g., in a Southern or Northern blot). Once the target glycosyltransferase nucleic acid is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York).

A glycosyltransferase nucleic acid can also be cloned by detecting its expressed product by means of assays based on the physical, chemical, or immunological properties. For example, one can identify a cloned glycosyltransferase nucleic acid by the ability of a polypeptide encoded by the nucleic acid to catalyze the transfer of a monosaccharide from a donor to an acceptor moiety. In a preferred method, capillary electrophoresis is employed to detect the reaction products. This highly sensitive assay involves using either monosaccharide or disaccharide aminophenyl derivatives which are labeled with fluorescein as described in Wakarchuk et al. (1996) J. Biol. Chem. 271 (45): 28271-276. For example, to assay for a Neisseria IgtC enzyme, either FCHASE-AP-Lac or FCHASE-AP-Gal can be used, whereas for the Neisseria IgtB enzyme an appropriate reagent is FCHASE-AP-GlcNAc (Id.).

As an alternative to cloning a glycosyltransferase gene, a glycosyltransferase nucleic acid can be chemically synthesized from a known sequence that encodes a glycosyltransferase. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is often limited to sequences of about 100 bases, longer sequences may be obtained by the ligation of shorter sequences.

Alternatively, subsequences can be cloned and the appropriate subsequences cleaved using appropriate restriction enzymes. The fragments may then be ligated to produce the desired DNA sequence.

In one embodiment, glycosyltransferase nucleic acids can be cloned using DNA amplification methods such as polymerase chain reaction (PCR). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired glycosyltransferase sequence or subsequence and having terminal restriction sites. This nucleic acid can then be easily ligated into a vector containing a nucleic acid encoding the second molecule and having the appropriate corresponding restriction sites. Suitable PCR primers can be determined by one of skill in the art using the sequence information provided in GenBank or other sources. Appropriate restriction sites can also be added to the nucleic acid encoding the glycosyltransferase protein or protein subsequence by site-directed mutagenesis. The plasmid containing the glycosyltransferase-encoding nucleotide sequence or subsequence is cleaved with the appropriate restriction endonuclease and then ligated into an appropriate vector for amplification and/or expression according to standard methods.

Other physical properties of a polypeptide expressed from a particular nucleic acid can be compared to properties of known glycosyltransferases to provide another method of identifying glycosyltransferase-encoding nucleic acids. Alternatively, a putative glycosyltransferase gene can be mutated, and its role as a glycosyltransferase established by detecting a variation in the structure of an oligosaccharide normally produced by the glycosyltransferase.

In some embodiments, it may be desirable to modify the glycosyltransferase or accessory enzyme nucleic acids. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

In a preferred embodiment, the recombinant nucleic acids present in the cells of the invention are modified to include preferred codons which enhance translation of the nucleic acid in a selected organism (e.g., yeast preferred codons are substituted into a coding nucleic acid for expression in yeast).

Nearly any glycosyltransferase can be used in the reaction mixtures and methods of the present invention. The appropriate sialyltransferase or glycosyltransferase is selected based upon the particular oligosaccharide or sialylated product saccharide that is desired.

The following list of sialyltransferases and glycosyltransferases is intended to be illustrative, but not limiting.

1. Sialyltransferases and Other Glycosyltransferases

Endogenous and exogenous sialyltransferases are useful in the recombinant cells of the invention. Cells that produce recombinant sialyltransferases will also produce CMP-sialic acid, which is a sialic acid donor for sialyltransferases. In preferred embodiments, bacterial sialyltransferases are used in the present invention. For example, α2,3-sialyltransferases have been isolated from Neisseria meningitides and Neisseria gonorrhea and are disclosed in U.S. Pat. Nos. 6,096,529, issued Aug. 1, 2000 and 6,210,933, issued Apr. 3, 2001; both of which are herein incorporated by reference for all purposes. α2,3-sialyltransferases and bifunctional α2,3-2,8-sialyltransferases have been isolated from Campylobacter jejuni and are disclosed in U.S. Pat. No. 6,699,705, issued Mar. 2, 2004, herein incorporated by reference for all purposes. Other bacterial sialyltransferases are known, e.g., from Haemophilus, for example Accession number X57315; and from Pasteurella multocida, for example Accession number AE006157. An ST6Gal II sialyltransferase from Photobacterium damsela has also been identified and can be used in the disclosed methods. (Yamamoto et al. (1996) J. Biochem. 120: 104-110)

Eukaryotic sialyltransferases can also be used in the invention. Examples of suitable eukaryotic sialyltransferases for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al. (1996) Glycobiology 6: v-xiv). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem., 256:3159 (1981), Weinstein et al., J. Biol. Chem., 257:13845 (1982) and Wen et al., J. Biol. Chem., 267:21011 (1992). Another exemplary α2,3-sialyltransferase (EC 2.4.99.4) transfers sialic acid to the non-reducing terminal Gal of the disaccharide or glycoside. See, Rearick et al., J. Biol. Chem., 254:4444 (1979) and Gillespie et al., J. Biol. Chem., 267:21004 (1992). Further exemplary enzymes include Gal-β-1,4-GlcNAc α-2,6 sialyltransferase (See, Kurosawa et al. Eur. J. Biochem. 219: 375-381 (1994)). Eukaryotic sialyltransferases generally comprise different functional domains, e.g., a cytoplasmic domain, a signal-anchor domain, a stem region and a catalytic domain. In preferred embodiments, the catalytic domain of a eukaryotic sialyltransferase is expressed in a host cell. Other sialyltransferases that can be used in the invention are found in Tables 3 and 4, below.

TABLE 3

| Sialyltransferase | Accession number |
|---|---|
| ST3Gal I | X73523 |
| ST3Gal II | BC015264 |
| ST3Gal II | X76989 |
| ST3Gal III | BC006710 |
| ST3Gal IV | BC011121 |
| ST3Gal V | AF119416 |
| ST3Gal VI | NM_018784 |
| ST6Gal I | BB768706 |
| ST6Gal I | BB768706 |
| ST6Gal I | D16106 |
| ST6GalNAc I | NM_011371 |
| ST6GalNAc I | NM_011371 |
| ST6GalNAc II | X93999 |
| ST6GalNAc III | Y11342 |
| ST6GalNAc IV | NM_011373 |
| ST6GalNAc IV | Y15779 |
| ST6GalNAc IV | Y15779 |
| ST6GalNAc V | AB028840 |
| ST6GalNAc VI | AB035123 |
| ST6GalNAc VI | AV101836 |
| ST6GalNAc VI | BB772604 |
| ST8Sia I | AW490593 |
| ST8Sia I | NM_011374 |
| ST8Sia II | X83562 |
| ST8Sia II | X83562 |
| ST8Sia III | X80502 |
| ST8Sia IV | X86000 |
| ST8Sia V | X98014 |
| ST8Sia VI | AB059554 |

TABLE 4

| Protein | | Organism | EC# | GenBank/GenPept | | SwissProt | PDB/3D |
|---|---|---|---|---|---|---|---|
| At1g08280 | | Arabidopsis | n.d. | AC011438 | AAF18241.1 | Q84W00 | |
| | | thaliana | | BT004583 | AAO42829.1 | Q9SGD2 | |
| | | | | NC_003070 | NP_172305.1 | | |
| At1g08660/F22O13.14 | | Arabidopsis | n.d. | AC003981 | AAF99778.1 | Q8VZJ0 | |
| | | thaliana | | AY064135 | AAL36042.1 | Q9FRR9 | |
| | | | | AY124807 | AAM70516.1 | | |
| | | | | NC_003070 | NP_172342.1 | | |
| | | | | NM_180609 | NP_850940.1 | | |
| At3g48820/T21J18_90 | | Arabidopsis | n.d. | AY080589 | AAL85966.1 | Q8RY00 | |
| | | thaliana | | AY133816 | AAM91750.1 | Q9M301 | |
| | | | | AL132963 | CAB87910.1 | | |
| | | | | NM_114741 | NP_190451.1 | | |
| α-2,3-sialyltransferase | | Bos taurus | n.d. | AJ584673 | CAE48298.1 | | |
| (ST3GAL-IV) | | | | | | | |
| α-2,3-sialyltransferase | | Bos taurus | n.d. | AJ585768 | CAE51392.1 | | |
| (St3Gal-V) | | | | | | | |
| α-2,6-sialyltransferase | | Bos taurus | n.d. | AJ620651 | CAF05850.1 | | |
| (Siat7b) | | | | | | | |
| α-2,8-sialyltransferase | | Bos taurus | 2.4.99.8 | AJ699418 | CAG27880.1 | | |
| (SIAT8A) | | | | | | | |
| α-2,8-sialyltransferase | | Bos taurus | n.d. | AJ699421 | CAG27883.1 | | |
| (Siat8D) | | | | | | | |
| α-2,8-sialyltransferase | | Bos taurus | n.d. | AJ704563 | CAG28696.1 | | |
| ST8Siα-III (Siat8C) | | | | | | | |
| CMP α-2,6- | | Bos taurus | 2.4.99.1 | Y15111 | CAA75385.1 | O18974 | |
| sialyltransferase (ST6Gal | | | | NM_177517 | NP_803483.1 | | |
| I) | | | | | | | |
| sialyltransferase 8 | | Bos taurus | n.d. | AF450088 | AAL47018.1 | Q8WN13 | |
| (fragment) | | | | | | | |
| sialyltransferase ST3Gal- | | Bos taurus | n.d. | AJ748841 | CAG44450.1 | | |
| II (Siat4B) | | | | | | | |
| sialyltransferase ST3Gal- | | Bos taurus | n.d. | AJ748842 | CAG44451.1 | | |
| III (Siat6) | | | | | | | |
| sialyltransferase ST3Gal- | | Bos taurus | n.d. | AJ748843 | CAG44452.1 | | |
| VI (Siat10) | | | | | | | |
| ST3Gal I | | Bos taurus | n.d. | AJ305086 | CAC24698.1 | Q9BEG4 | |
| St6GalNAc-VI | | Bos taurus | n.d. | AJ620949 | CAF06586.1 | | |
| CDS4 | | Branchiostoma | n.d. | AF391289 | AAM18873.1 | Q8T771 | |
| | | floridae | | | | | |
| polysialyltransferase | | Cercopithecus | 2.4.99.— | AF210729 | AAF17105.1 | Q9TT09 | |
| (PST) (fragment) ST8Sia | | aethiops | | | | | |
| IV | | | | | | | |
| polysialyltransferase | | Cercopithecus | 2.4.99.— | AF210318 | AAF17104.1 | Q9TT10 | |
| (STX) (fragment) ST8Sia | | aethiops | | | | | |
| II | | | | | | | |
| α-2,3-sialyltransferase | | Ciona intestinalis | n.d. | AJ626815 | CAF25173.1 | | |
| ST3Gal I (Siat4) | | | | | | | |
| α-2,3-sialyltransferase | | Ciona savignyi | n.d. | AJ626814 | CAF25172.1 | | |
| ST3Gal I (Siat4) | | | | | | | |
| α-2,8- | | Cricetulus griseus | 2.4.99.— | — | AAE28634 | Q64690 | |
| polysialyltransferase | | | | Z46801 | CAA86822.1 | | |
| ST8Sia IV | | | | | | | |
| Gal β-1,3/4-GlcNAc α- | | Cricetulus griseus | n.d. | AY266675 | AAP22942.1 | Q80WL0 | |
| 2,3-sialyltransferase | | | | | | | |
| St3Gal I | | | | | | | |
| Gal β-1,3/4-GlcNAc α- | | Cricetulus griseus | n.d. | AY266676 | AAP22943.1 | Q80WK9 | |
| 2,3-sialyltransferase | | | | | | | |
| St3Gal II (fragment) | | | | | | | |
| α-2,3-sialyltransferase | | Danio rerio | n.d. | AJ783740 | CAH04017.1 | | |
| ST3Gal I (Siat4) | | | | | | | |
| α-2,3-sialyltransferase | | Danio rerio | n.d. | AJ783741 | CAH04018.1 | | |
| ST3Gal II (Siat5) | | | | | | | |
| α-2,3-sialyltransferase | | Danio rerio | n.d. | AJ626821 | CAF25179.1 | | |
| ST3Gal III (Siat6) | | | | | | | |
| α-2,3-sialyltransferase | | Danio rerio | n.d. | AJ744809 | CAG32845.1 | | |
| ST3Gal IV (Siat4c) | | | | | | | |
| α-2,3-sialyltransferase | | Danio rerio | n.d. | AJ783742 | CAH04019.1 | | |
| ST3Gal V-r (Siat5-related) | | | | | | | |
| α-2,6-sialyltransferase | | Danio rerio | n.d. | AJ744801 | CAG32837.1 | | |
| ST6Gal I (Siat1) | | | | | | | |
| α-2,6-sialyltransferase | | Danio rerio | n.d. | AJ634459 | CAG25680.1 | | |
| ST6GalNAc II (Siat7B) | | | | | | | |
| α-2,6-sialyltransferase | | Danio rerio | n.d. | AJ646874 | CAG26703.1 | | |
| ST6GalNAc V (Siat7E) | | | | | | | |
| (fragment) | | | | | | | |
| α-2,6-sialyltransferase | | Danio rerio | n.d. | AJ646883 | CAG26712.1 | | |
| ST6GalNAc VI (Siat7F) | | | | | | | |
| (fragment) | | | | | | | |
| α-2,8-sialyltransferase | | Danio rerio | n.d. | AJ715535 | CAG29374.1 | | |
| ST8Sia I (Siat 8A) | | | | | | | |
| (fragment) | | | | | | | |
| α-2,8-sialyltransferase | | Danio rerio | n.d. | AJ715543 | CAG29382.1 | | |
| ST8Sia III (Siat 8C) | | | | | | | |
| (fragment) | | | | | | | |
| α-2,8-sialyltransferase | | Danio rerio | n.d. | AJ715545 | CAG29384.1 | | |
| ST8Sia IV (Siat 8D) | | | | | | | |
| (fragment) | | | | | | | |
| α-2,8-sialyltransferase | | Danio rerio | n.d. | AJ715546 | CAG29385.1 | | |
| ST8SiaV (Siat 8E) | | | | | | | |
| (fragment) | | | | | | | |
| α-2,8-sialyltransferase | | Danio rerio | n.d. | AJ715551 | CAG29390.1 | | |
| ST8Sia VI (Siat 8F) | | | | | | | |
| (fragment) | | | | | | | |
| β-galactosamide α-2,6- | | Danio rerio | n.d. | AJ627627 | CAF29495.1 | | |
| sialyltransferase II | | | | | | | |
| (ST6Gal II) | | | | | | | |
| N-glycan α-2,8- | | Danio rerio | n.d. | BC050483 | AAH50483.1 | Q7ZU51 | |
| sialyltransferase | | | | AY055462 | AAL17875.1 | Q8QH83 | |
| | | | | NM_153662 | NP_705948.1 | | |
| ST3Gal III-related (siat6r) | | Danio rerio | n.d. | BC053179 | AAH53179.1 | Q7T3B9 | |
| | | | | AJ626820 | CAF25178.1 | | |
| | | | | NM_200355 | NP_956649.1 | | |
| St3Gal-V | | Danio rerio | n.d. | AJ619960 | CAF04061.1 | | |
| st6GalNAc-VI | | Danio rerio | n.d. | BC060932 | AAH60932.1 | | |
| | | | | AJ620947 | CAF06584.1 | | |
| α-2,6-sialyltransferase | | Drosophila | 2.4.99.1 | AE003465 | AAF47256.1 | Q9GU23 | |
| (CG4871) ST6Gal I | | melanogaster | | AF218237 | AAG13185.1 | Q9W121 | |
| | | | | AF397532 | AAK92126.1 | | |
| | | | | AE003465 | AAM70791.1 | | |
| | | | | NM_079129 | NP_523853.1 | | |
| | | | | NM_166684 | NP_726474.1 | | |
| α-2,3-sialyltransferase | | Gallus gallus | n.d. | AJ585767 | CAE51391.1 | | |
| (ST3Gal-VI) | | | | AJ627204 | CAF25503.1 | | |
| α-2,3-sialyltransferase | | Gallus gallus | 2.4.99.4 | X80503 | CAA56666.1 | Q11200 | |
| ST3Gal I | | | | NM_205217 | NP_990548.1 | | |
| α-2,3-sialyltransferase | | Gallus gallus | 2.4.99.— | AF035250 | AAC14163.1 | O73724 | |
| ST3Gal IV (fragment) | | | | | | | |
| α-2,3-sialytransferase | | Gallus gallus | n.d. | AJ585761 | CAE51385.2 | | |
| (ST3GAL-II) | | | | | | | |
| α-2,6-sialyltransferase | | Gallus gallus | n.d. | AJ620653 | CAF05852.1 | | |
| (Siat7b) | | | | | | | |
| α-2,6-sialyltransferase | | Gallus gallus | 2.4.99.1 | X75558 | CAA53235.1 | Q92182 | |
| ST6Gal I | | | | NM_205241 | NP_990572.1 | | |
| α-2,6-sialyltransferase | | Gallus gallus | 2.4.99.3 | — | AAE68028.1 | Q92183 | |
| ST6GalNAc I | | | | — | AAE68029.1 | | |
| | | | | X74946 | CAA52902.1 | | |
| | | | | NM_205240 | NP_990571.1 | | |
| α-2,6-sialyltransferase | | Gallus gallus | 2.4.99.— | X77775 | AAE68030.1 | Q92184 | |
| ST6GalNAc II | | | | NM_205233 | CAA54813.1 | | |
| | | | | | NP_990564.1 | | |
| α-2,6-sialyltransferase | | Gallus gallus | n.d. | AJ634455 | CAG25677.1 | | |
| ST6GalNAc III (SIAT7C) | | | | | | | |
| (fragment) | | | | | | | |
| α-2,6-sialyltransferase | | Gallus gallus | n.d. | AJ646877 | CAG26706.1 | | |
| ST6GalNAc V (SIAT7E) | | | | | | | |
| (fragment) | | | | | | | |
| α-2,8-sialyltransferase | | Gallus gallus | 2.4.99.— | U73176 | AAC28888.1 | P79783 | |
| (GD3 Synthase) ST8Sia I | | | | | | | |
| α-2,8-sialyltransferase | | Gallus gallus | n.d. | AJ699419 | CAG27881.1 | | |
| (SIAT8B) | | | | | | | |
| α-2,8-sialyltransferase | | Gallus gallus | n.d. | AJ699420 | CAG27882.1 | | |
| (SIAT8C) | | | | | | | |
| α-2,8-sialyltransferase | | Gallus gallus | n.d. | AJ699424 | CAG27886.1 | | |
| (SIAT8F) | | | | | | | |
| α-2,8-syalyltransferase | | Gallus gallus | n.d. | AJ704564 | CAG28697.1 | | |
| ST8Siα-V (SIAT8C) | | | | | | | |
| β-galactosamide α-2,6- | | Gallus gallus | n.d. | AJ627629 | CAF29497.1 | | |
| sialyltransferase II | | | | | | | |
| (ST6Gal II) | | | | | | | |
| GM3 synthase (SIAT9) | | Gallus gallus | 2.4.99.9 | AY515255 | AAS83519.1 | | |
| polysialyltransferase | | Gallus gallus | 2.4.99.— | AF008194 | AAB95120.1 | O42399 | |
| ST8Sia IV | | | | | | | |
| α-2,3-sialyltransferase | | Homo sapiens | 2.4.99.4 | L29555 | AAA36612.1 | Q11201 | |
| ST3Gal I | | | | AF059321 | AAC17874.1 | O60677 | |
| | | | | L13972 | AAC37574.1 | Q9UN51 | |
| | | | | AF155238 | AAD39238.1 | | |
| | | | | AF186191 | AAG29876.1 | | |
| | | | | BC018357 | AAH18357.1 | | |
| | | | | NM_003033 | NP_003024.1 | | |
| | | | | NM_173344 | NP_775479.1 | | |
| α-2,3-sialyltransferase | | Homo sapiens | 2.4.99.4 | U63090 | AAB40389.1 | Q16842 | |
| ST3Gal II | | | | BC036777 | AAH36777.1 | O00654 | |
| | | | | X96667 | CAA65447.1 | | |
| | | | | NM_006927 | NP_008858.1 | | |
| α-2,3-sialyltransferase | | Homo sapiens | 2.4.99.6 | L23768 | AAA35778.1 | Q11203 | |
| ST3Gal III (SiaT6) | | | | BC050380 | AAH50380.1 | Q86UR6 | |
| | | | | AF425851 | AAO13859.1 | Q86UR7 | |
| | | | | AF425852 | AAO13860.1 | Q86UR8 | |
| | | | | AF425853 | AAO13861.1 | Q86UR9 | |
| | | | | AF425854 | AAO13862.1 | Q86US0 | |
| | | | | AF425855 | AAO13863.1 | Q86US1 | |
| | | | | AF425856 | AAO13864.1 | Q86US2 | |
| | | | | AF425857 | AAO13865.1 | Q8IX43 | |
| | | | | AF425858 | AAO13866.1 | Q8IX44 | |
| | | | | AF425859 | AAO13867.1 | Q8IX45 | |
| | | | | AF425860 | AAO13868.1 | Q8IX46 | |
| | | | | AF425861 | AAO13869.1 | Q8IX47 | |
| | | | | AF425862 | AAO13870.1 | Q8IX48 | |
| | | | | AF425863 | AAO13871.1 | Q8IX49 | |
| | | | | AF425864 | AAO13872.1 | Q8IX50 | |
| | | | | AF425865 | AAO13873.1 | Q8IX51 | |
| | | | | AF425866 | AAO13874.1 | Q8IX52 | |
| | | | | AF425867 | AAO13875.1 | Q8IX53 | |
| | | | | AY167992 | AAO38806.1 | Q8IX54 | |
| | | | | AY167993 | AAO38807.1 | Q8IX55 | |
| | | | | AY167994 | AAO38808.1 | Q8IX56 | |
| | | | | AY167995 | AAO38809.1 | Q8IX57 | |
| | | | | AY167996 | AAO38810.1 | Q8IX58 | |
| | | | | AY167997 | AAO38811.1 | | |
| | | | | AY167998 | AAO38812.1 | | |
| | | | | NM_006279 | NP_006270.1 | | |
| | | | | NM_174964 | NP_777624.1 | | |
| | | | | NM_174965 | NP_777625.1 | | |
| | | | | NM_174966 | NP_777626.1 | | |
| | | | | NM_174967 | NP_777627.1 | | |
| | | | | NM_174969 | NP_777629.1 | | |
| | | | | NM_174970 | NP_777630.1 | | |
| | | | | NM_174972 | NP_777632.1 | | |
| α-2,3-sialyltransferase | | Homo sapiens | 2.4.99.— | L23767 | AAA16460.1 | Q11206 | |
| ST3Gal IV | | | | AF035249 | AAC14162.1 | O60497 | |
| | | | | BC010645 | AAH10645.1 | Q96QQ9 | |
| | | | | AY040826 | AAK93790.1 | Q8N6A6 | |
| | | | | AF516602 | AAM66431.1 | Q8N6A7 | |
| | | | | AF516603 | AAM66432.1 | Q8NFD3 | |
| | | | | AF516604 | AAM66433.1 | Q8NFG7 | |
| | | | | AF525084 | AAM81378.1 | | |
| | | | | X74570 | CAA52662.1 | | |
| | | | | CR456858 | CAG33139.1 | | |
| | | | | NM_006278 | NP_006269.1 | | |
| α-2,3-sialyltransferase | | Homo sapiens | 2.4.99.4 | AF119391 | AAD39131.1 | Q9Y274 | |
| ST3Gal VI | | | | BC023312 | AAH23312.1 | | |
| | | | | AB022918 | BAA77609.1 | | |
| | | | | AX877828 | CAE89895.1 | | |
| | | | | AX886023 | CAF00161.1 | | |
| | | | | NM_006100 | NP_006091.1 | | |
| α-2,6-sialyltransferase | | Homo sapiens | n.d. | BC008680 | AAH08680.1 | Q86Y44 | |
| (ST6Gal II; KIAA1877) | | | | AB058780 | BAB47506.1 | Q8IUG7 | |
| | | | | AB059555 | BAC24793.1 | Q96HE4 | |
| | | | | AJ512141 | CAD54408.1 | Q96JF0 | |
| | | | | AX795193 | CAE48260.1 | | |
| | | | | AX795193 | CAE48261.1 | | |
| | | | | NM_032528 | NP_115917.1 | | |
| α-2,6-sialyltransferase | | Homo sapiens | n.d. | BC059363 | AAH59363.1 | Q8N259 | |
| (ST6GALNAC III) | | | | AY358540 | AAQ88904.1 | Q8NDV1 | |
| | | | | AK091215 | BAC03611.1 | | |
| | | | | AJ507291 | CAD45371.1 | | |
| | | | | NM_152996 | NP_694541.1 | | |
| α-2,6-sialyltransferase | | Homo sapiens | n.d. | BC001201 | AAH01201.1 | Q9BVH7 | |
| (ST6GalNAc V) | | | | AK056241 | BAB71127.1 | | |
| | | | | AL035409 | CAB72344.1 | | |
| | | | | AJ507292 | CAD45372.1 | | |
| | | | | NM_030965 | NP_112227.1 | | |
| α-2,6-sialyltransferase | | Homo sapiens | 2.4.99.— | U14550 | AAA52228.1 | Q9UJ37 | |
| (SThM) ST6GalNAc II | | | | BC040455 | AAH40455.1 | Q12971 | |
| | | | | AJ251053 | CAB61434.1 | | |
| | | | | NM_006456 | NP_006447.1 | | |
| α-2,6-sialyltransferase | | Homo sapiens | 2.4.99.1 | BC031476 | AAH31476.1 | P15907 | |
| ST6Gal I | | | | BC040009 | AAH40009.1 | | |
| | | | | A17362 | CAA01327.1 | | |
| | | | | A23699 | CAA01686.1 | | |
| | | | | X17247 | CAA35111.1 | | |
| | | | | X54363 | CAA38246.1 | | |
| | | | | X62822 | CAA44634.1 | | |
| | | | | NM_003032 | NP_003023.1 | | |
| | | | | NM_173216 | NP_775323.1 | | |
| α-2,6-sialyltransferase | | Homo sapiens | 2.4.99.3 | BC022462 | AAH22462.1 | Q8TBJ6 | |
| ST6GalNAc I | | | | AY096001 | AAM22800.1 | Q9NSC7 | |
| | | | | AY358918 | AAQ89277.1 | Q9NXQ7 | |
| | | | | AK000113 | BAA90953.1 | | |
| | | | | Y11339 | CAA72179.2 | | |
| | | | | NM_018414 | NP_060884.1 | | |
| α-2,8- | | Homo sapiens | 2.4.99.— | L41680 | AAC41775.1 | Q8N1F4 | |
| polysialyltransferase | | | | BC027866 | AAH27866.1 | Q92187 | |
| ST8Sia IV | | | | BC053657 | AAH53657.1 | Q92693 | |
| | | | | NM_005668 | NP_005659.1 | | |
| α-2,8-sialyltransferase | | Homo sapiens | 2.4.99.8 | L32867 | AAA62366.1 | Q86X71 | |
| (GD3 synthase) ST8Sia I | | | | L43494 | AAC37586.1 | Q92185 | |
| | | | | BC046158 | AAH46158.1 | Q93064 | |
| | | | | — | AAQ53140.1 | | |
| | | | | AY569975 | AAS75783.1 | | |
| | | | | D26360 | BAA05391.1 | | |
| | | | | X77922 | CAA54891.1 | | |
| | | | | NM_003034 | NP_003025.1 | | |
| α-2,8-sialyltransferase | | Homo sapiens | 2.4.99.— | L29556 | AAA36613.1 | Q92186 | |
| ST8Sia II | | | | U82762 | AAB51242.1 | Q92470 | |
| | | | | U33551 | AAC24458.1 | Q92746 | |
| | | | | BC069584 | AAH69584.1 | | |
| | | | | NM_006011 | NP_006002.1 | | |
| α-2,8-sialyltransferase | | Homo sapiens | 2.4.99.— | AF004668 | AAB87642.1 | O43173 | |
| ST8Sia III | | | | AF003092 | AAC15901.2 | Q9NS41 | |
| | | | | NM_015879 | NP_056963.1 | | |
| α-2,8-sialyltransferase | | Homo sapiens | 2.4.99.— | U91641 | AAC51727.1 | O15466 | |
| ST8Sia V | | | | CR457037 | CAG33318.1 | | |
| | | | | NM_013305 | NP_037437.1 | | |
| ENSP00000020221 | | Homo sapiens | n.d. | AC023295 | — | | |
| (fragment) | | | | | | | |
| lactosylceramide α-2,3- | | Homo sapiens | 2.4.99.9 | AF105026 | AAD14634.1 | Q9UNP4 | |
| sialyltransferase (ST3Gal | | | | AF119415 | AAF66146.1 | O94902 | |
| V) | | | | BC065936 | AAH65936.1 | | |
| | | | | AY152815 | AAO16866.1 | | |
| | | | | AAP65066 | AAP65066.1 | | |
| | | | | AY359105 | AAQ89463.1 | | |
| | | | | AB018356 | BAA33950.1 | | |
| | | | | AX876536 | CAE89320.1 | | |
| | | | | NM_003896 | NP_003887.2 | | |
| N-acetylgalactosaminide | | Homo sapiens | 2.4.99.— | BC006564 | AAH06564.1 | Q969X2 | |
| α-2,6-sialyltransferase | | | | BC007802 | AAH07802.1 | Q9H8A2 | |
| (ST6GalNAc VI) | | | | BC016299 | AAH16299.1 | Q9ULB8 | |
| | | | | AY358672 | AAQ89035.1 | | |
| | | | | AB035173 | BAA87035.1 | | |
| | | | | AK023900 | BAB14715.1 | | |
| | | | | AJ507293 | CAD45373.1 | | |
| | | | | AX880950 | CAE91145.1 | | |
| | | | | CR457318 | CAG33599.1 | | |
| | | | | NM_013443 | NP_038471.2 | | |
| N-acetylgalactosaminide | | Homo sapiens | 2.4.99.— | AF127142 | AAF00102.1 | Q9H4F1 | |
| α-2,6-sialyltransferase IV | | | | BC036705 | AAH36705.1 | Q9NWU6 | |
| (ST6GalNAc IV) | | | | — | AAP63349.1 | Q9UKU1 | |
| | | | | AB035172 | BAA87034.1 | Q9ULB9 | |
| | | | | AK000600 | BAA91281.1 | Q9Y3G3 | |
| | | | | Y17461 | CAB44354.1 | Q9Y3G4 | |
| | | | | AJ271734 | CAC07404.1 | | |
| | | | | AX061620 | CAC24981.1 | | |
| | | | | AX068265 | CAC27250.1 | | |
| | | | | AX969252 | CAF14360.1 | | |
| | | | | NM_014403 | NP_055218.3 | | |
| | | | | NM_175039 | NP_778204.1 | | |
| ST8SIA-VI (fragment) | | Homo sapiens | n.d. | AJ621583 | CAF21722.1 | | |
| | | | | XM_291725 | XP_291725.2 | | |
| unnamed protein product | | Homo sapiens | n.d. | AK021929 | BAB13940.1 | Q9HAA9 | |
| | | | | AX881696 | CAE91353.1 | | |
| Gal β-1,3/4-GlcNAc α- | | Mesocricetus | 2.4.99.6 | AJ245699 | CAB53394.1 | Q9QXF6 | |
| 2,3-sialyltransferase | | auratus | | | | | |
| (ST3Gal III) | | | | | | | |
| Gal β-1,3/4-GlcNAc α- | | Mesocricetus | 2.4.99.6 | AJ245700 | CAB53395.1 | Q9QXF5 | |
| 2,3-sialyltransferase | | auratus | | | | | |
| (ST3Gal IV) | | | | | | | |
| GD3 synthase (fragment) | | Mesocricetus | n.d. | AF141657 | AAD33879.1 | Q9WUL1 | |
| ST8Sia I | | auratus | | | | | |
| polysialyltransferase | | Mesocricetus | 2.4.99.— | AJ245701 | CAB53396.1 | Q9QXF4 | |
| (ST8Sia IV) | | auratus | | | | | |
| α-2,3-sialyltransferase | St3gal1 | Mus musculus | 2.4.99.4 | AF214028 | AAF60973.1 | P54751 | |
| ST3Gal I | | | | AK031344 | BAC27356.1 | Q11202 | |
| | | | | AK078469 | BAC37290.1 | Q9JL30 | |
| | | | | X73523 | CAA51919.1 | | |
| | | | | NM_009177 | NP_033203.1 | | |
| α-2,3-sialyltransferase | St3gal2 | Mus musculus | 2.4.99.4 | BC015264 | AAH15264.1 | Q11204 | |
| ST3Gal II | | | | BC066064 | AAH66064.1 | Q8BPL0 | |
| | | | | AK034554 | BAC28752.1 | Q8BSA0 | |
| | | | | AK034863 | BAC28859.1 | Q8BSE9 | |
| | | | | AK053827 | BAC35543.1 | Q91WH6 | |
| | | | | X76989 | CAA54294.1 | | |
| | | | | NM_009179 | NP_033205.1 | | |
| | | | | NM_178048 | NP_835149.1 | | |
| α-2,3-sialyltransferase | St3gal3 | Mus musculus | 2.4.99.— | BC006710 | AAH06710.1 | P97325 | |
| ST3Gal III | | | | AK005053 | BAB23779.1 | Q922X5 | |
| | | | | AK013016 | BAB28598.1 | Q9CZ48 | |
| | | | | X84234 | CAA59013.1 | Q9DBB6 | |
| | | | | NM_009176 | NP_033202.2 | | |
| α-2,3-sialyltransferase | St3gal4 | Mus musculus | 2.4.99.4 | BC011121 | AAH11121.1 | P97354 | |
| ST3Gal IV | | | | BC050773 | AAH50773.1 | Q61325 | |
| | | | | D28941 | BAA06068.1 | Q91Y74 | |
| | | | | AK008543 | BAB25732.1 | Q921R5 | |
| | | | | AK061305 | BAB47508.1 | Q9CVE8 | |
| | | | | X95809 | CAA65076.1 | | |
| | | | | NM_009178 | NP_033204.2 | | |
| α-2,3-sialyltransferase | St3gal6 | Mus musculus | 2.4.99.4 | AF119390 | AAD39130.1 | Q80UR7 | |
| ST3Gal VI | | | | BC052338 | AAH52338.1 | Q8BLV1 | |
| | | | | AB063326 | BAB79494.1 | Q8VIB3 | |
| | | | | AK033562 | BAC28360.1 | Q9WVG2 | |
| | | | | AK041173 | BAC30851.1 | | |
| | | | | NM_018784 | NP_061254 | | |
| α-2,6-sialyltransferase | St6galnac2 | Mus musculus | 2.4.99.— | NM_009180 | 6677963 | P70277 | |
| ST6GalNAc II | | | | BC010208 | AAH10208.1 | Q9DC24 | |
| | | | | AB027198 | BAB00637.1 | Q9JJM5 | |
| | | | | AK004613 | BAB23410.1 | | |
| | | | | X93999 | CAA63821.1 | | |
| | | | | X94000 | CAA63822.1 | | |
| | | | | NM_009180 | NP_033206.2 | | |
| α-2,6-sialyltransferase | St6gal1 | Mus musculus | 2.4.99.1 | — | AAE68031.1 | Q64685 | |
| ST6Gal I | | | | BC027833 | AAH27833.1 | Q8BM62 | |
| | | | | D16106 | BAA03680.1 | Q8K1L1 | |
| | | | | AK034768 | BAC28828.1 | | |
| | | | | AK084124 | BAC39120.1 | | |
| | | | | NM_145933 | NP_666045.1 | | |
| α-2,6-sialyltransferase | St6gal2 | Mus musculus | n.d. | AK082566 | BAC38534.1 | Q8BUU4 | |
| ST6Gal II | | | | AB095093 | BAC87752.1 | | |
| | | | | AK129462 | BAC98272.1 | | |
| | | | | NM_172829 | NP_766417.1 | | |
| α-2,6-sialyltransferase | St6galnac1 | Mus musculus | 2.4.99.3 | Y11274 | CAA72137.1 | Q9QZ39 | |
| ST6GalNAc I | | | | NM_011371 | NP_035501.1 | Q9JJP5 | |
| α-2,6-sialyltransferase | St6galnac3 | Mus musculus | n.d. | BC058387 | AAH58387.1 | Q9WUV2 | |
| ST6GalNAc III | | | | AK034804 | BAC28836.1 | Q9JHP5 | |
| | | | | Y11342 | CAA72181.2 | | |
| | | | | Y11343 | CAB95031.1 | | |
| | | | | NM_011372 | NP_035502 | | |
| α-2,6-sialyltransferase | St6galnac4 | Mus musculus | 2.4.99.7 | BC056451 | AAH56451.1 | Q8C3J2 | |
| ST6GalNAc IV | | | | AK085730 | BAC39523.1 | Q9JHP2 | |
| | | | | AJ007310 | CAA07446.1 | Q9R2B6 | |
| | | | | Y15779 | CAB43507.1 | O88725 | |
| | | | | Y15780 | CAB43514.1 | Q9JHP0 | |
| | | | | Y19055 | CAB93946.1 | Q9QUP9 | |
| | | | | Y19057 | CAB93948.1 | Q9R2B5 | |
| | | | | NM_011373 | NP_035503.1 | | |
| α-2,8-sialyltransferase | St8sia1 | Mus musculus | 2.4.99.8 | L38677 | AAA91869.1 | Q64468 | |
| (GD3 synthase) ST8Sia I | | | | BC024821 | AAH24821.1 | Q64687 | |
| | | | | AK046188 | BAC32625.1 | Q8BL76 | |
| | | | | AK052444 | BAC34994.1 | Q8BWI0 | |
| | | | | X84235 | CAA59014.1 | Q8K1C1 | |
| | | | | AJ401102 | CAC20706.1 | Q9EPK0 | |
| | | | | NM_011374 | NP_035504.1 | | |
| α-2,8-sialyltransferase | St8sia6 | Mus musculus | n.d. | AB059554 | BAC01265.1 | Q8BI43 | |
| (ST8Sia VI) | | | | AK085105 | BAC39367.1 | Q8K4T1 | |
| | | | | NM_145838 | NP_665837.1 | | |
| α-2,8-sialyltransferase | St8sia2 | Mus musculus | 2.4.99.— | X83562 | CAA58548.1 | O35696 | |
| ST8Sia II | | | | X99646 | CAA67965.1 | | |
| | | | | X99647 | CAA67965.1 | | |
| | | | | X99648 | CAA67965.1 | | |
| | | | | X99649 | CAA67965.1 | | |
| | | | | X99650 | CAA67965.1 | | |
| | | | | X99651 | CAA67965.1 | | |
| | | | | NM_009181 | NP_033207.1 | | |
| α-2,8-sialyltransferase | St8sia4 | Mus musculus | 2.4.99.8 | BC060112 | AAH60112.1 | Q64692 | |
| ST8Sia IV | | | | AK003690 | BAB22941.1 | Q8BY70 | |
| | | | | AK041723 | BAC31044.1 | | |
| | | | | AJ223956 | CAA11685.1 | | |
| | | | | X86000 | CAA59992.1 | | |
| | | | | Y09484 | CAA70692.1 | | |
| | | | | NM_009183 | NP_033209.1 | | |
| α-2,8-sialyltransferase | St8sia5 | Mus musculus | 2.4.99.— | BC034855 | AAH34855.1 | P70126 | |
| ST8Sia V | | | | AK078670 | BAC37354.1 | P70127 | |
| | | | | X98014 | CAA66642.1 | P70128 | |
| | | | | X98014 | CAA66643.1 | Q8BJW0 | |
| | | | | X98014 | CAA66644.1 | Q8JZQ3 | |
| | | | | NM_013666 | NP_038694.1 | | |
| | | | | NM_153124 | NP_694764.1 | | |
| | | | | NM_177416 | NP_803135.1 | | |
| α-2,8-sialytransferase | St8sia3 | Mus musculus | 2.4.99.— | BC075645 | AAH75645.1 | Q64689 | |
| ST8Sia III | | | | AK015874 | BAB30012.1 | Q9CUJ6 | |
| | | | | X80502 | CAA56665.1 | | |
| | | | | NM_009182 | NP_033208.1 | | |
| GD1 synthase | St6galnac5 | Mus musculus | n.d. | BC055737 | AAH55737.1 | Q8CAM7 | |
| (ST6GalNAc V) | | | | AB030836 | BAA85747.1 | Q8CBX1 | |
| | | | | AB028840 | BAA89292.1 | Q9QYJ1 | |
| | | | | AK034387 | BAC28693.1 | Q9R0K6 | |
| | | | | AK038434 | BAC29997.1 | | |
| | | | | AK042683 | BAC31331.1 | | |
| | | | | NM_012028 | NP_036158.2 | | |
| GM3 synthase (α-2,3- | St3gal5 | Mus musculus | 2.4.99.9 | AF119416 | AAF66147.1 | O88829 | |
| sialyltransferase) ST3Gal V | | | | — | AAP65063.1 | Q9CZ65 | |
| | | | | AB018048 | BAA33491.1 | Q9QWF9 | |
| | | | | AB013302 | BAA76467.1 | | |
| | | | | AK012961 | BAB28571.1 | | |
| | | | | Y15003 | CAA75235.1 | | |
| | | | | NM_011375 | NP_035505.1 | | |
| N-acetylgalactosaminide | St6galnac6 | Mus musculus | 2.4.99.— | BC036985 | AAH36985.1 | Q8CDC3 | |
| α-2,6-sialyltransferase | | | | AB035174 | BAA87036.1 | Q8JZW3 | |
| (ST6GalNAc VI) | | | | AB035123 | BAA95940.1 | Q9JM95 | |
| | | | | AK030648 | BAC27064.1 | Q9R0G9 | |
| | | | | NM_016973 | NP_058669.1 | | |
| M138L | | Myxoma virus | n.d. | U46578 | AAD00069.1 | | |
| | | | | AF170726 | AAE61323.1 | | |
| | | | | NC_001132 | AAE61326.1 | | |
| | | | | | AAF15026.1 | | |
| | | | | | NP_051852.1 | | |
| α-2,3-sialyltransferase | | Oncorhynchus | n.d. | AJ585760 | CAE51384.1 | | |
| (St3Gal-I) | | mykiss | | | | | |
| α-2,6-sialyltransferase | | Oncorhynchus | n.d. | AJ620649 | CAF05848.1 | | |
| (Siat1) | | mykiss | | | | | |
| α-2,8- | | Oncorhynchus | n.d. | AB094402 | BAC77411.1 | Q7T2X5 | |
| polysialyltransferase IV | | mykiss | | | | | |
| (ST8Sia IV) | | | | | | | |
| GalNAc α-2,6- | | Oncorhynchus | n.d. | AB097943 | BAC77520.1 | Q7T2X4 | |
| sialyltransferase | | mykiss | | | | | |
| (RtST6GalNAc) | | | | | | | |
| α-2,3-sialyltransferase | | Oryctolagus | 2.4.99.— | AF121967 | AAF28871.1 | Q9N257 | |
| ST3Gal IV | | cuniculus | | | | | |
| OJ1217_F02.7 | | Oryza sativa | n.d. | AP004084 | BAD07616.1 | | |
| | | (japonica cultivar- | | | | | |
| | | group) | | | | | |
| OSJNBa0043L24.2 or | | Oryza sativa | n.d. | AL731626 | CAD41185.1 | | |
| OSJNBb0002J11.9 | | (japonica cultivar- | | AL662969 | CAE04714.1 | | |
| | | group) | | | | | |
| P0683f02.18 or | | Oryza sativa | n.d. | AP003289 | BAB63715.1 | | |
| P0489B03.1 | | (japonica cultivar- | | AP003794 | BAB90552.1 | | |
| | | group) | | | | | |
| α-2,6-sialyltransferase | | Oryzias latipes | n.d. | AJ646876 | CAG26705.1 | | |
| ST6GalNAc V (Siat7E) | | | | | | | |
| (fragment) | | | | | | | |
| α-2,3-sialyltransferase | | Pan troglodytes | n.d. | AJ744803 | CAG32839.1 | | |
| ST3Gal I (Siat4) | | | | | | | |
| α-2,3-sialyltransferase | | Pan troglodytes | n.d. | AJ744804 | CAG32840.1 | | |
| ST3Gal II (Siat5) | | | | | | | |
| α-2,3-sialyltransferase | | Pan troglodytes | n.d. | AJ626819 | CAF25177.1 | | |
| ST3Gal III (Siat6) | | | | | | | |
| α-2,3-sialyltransferase | | Pan troglodytes | n.d. | AJ626824 | CAF25182.1 | | |
| ST3Gal IV (Siat4c) | | | | | | | |
| α-2,3-sialyltransferase | | Pan troglodytes | n.d. | AJ744808 | CAG32844.1 | | |
| ST3Gal VI (Siat10) | | | | | | | |
| α-2,6-sialyltransferase | | Pan troglodytes | n.d. | AJ748740 | CAG38615.1 | | |
| (Sia7A) | | | | | | | |
| α-2,6-sialyltransferase | | Pan troglodytes | n.d. | AJ748741 | CAG38616.1 | | |
| (Sia7B) | | | | | | | |
| α-2,6-sialyltransferase | | Pan troglodytes | n.d. | AJ634454 | CAG25676.1 | | |
| ST6GalNAc III (Siat7C) | | | | | | | |
| α-2,6-sialyltransferase | | Pan troglodytes | n.d. | AJ646870 | CAG26699.1 | | |
| ST6GalNAc IV (Siat7D) | | | | | | | |
| (fragment) | | | | | | | |
| α-2,6-sialyltransferase | | Pan troglodytes | n.d. | AJ646875 | CAG26704.1 | | |
| ST6GalNAc V (Siat7E) | | | | | | | |
| α-2,6-sialyltransferase | | Pan troglodytes | n.d. | AJ646882 | CAG26711.1 | | |
| ST6GalNAc VI (Siat7F) | | | | | | | |
| (fragment) | | | | | | | |
| α-2,8-sialyltransferase | | Pan troglodytes | 2.4.99.8 | AJ697658 | CAG26896.1 | | |
| 8A (Siat8A) | | | | | | | |
| α-2,8-sialyltransferase | | Pan troglodytes | n.d. | AJ697659 | CAG26897.1 | | |
| 8B (Siat8B) | | | | | | | |
| α-2,8-sialyltransferase | | Pan troglodytes | n.d. | AJ697660 | CAG26898.1 | | |
| 8C (Siat8C) | | | | | | | |
| α-2,8-sialyltransferase | | Pan troglodytes | n.d. | AJ697661 | CAG26899.1 | | |
| 8D (Siat8D) | | | | | | | |
| α-2,8-sialyltransferase | | Pan troglodytes | n.d. | AJ697662 | CAG26900.1 | | |
| 8E (Siat8E) | | | | | | | |
| α-2,8-sialyltransferase | | Pan troglodytes | n.d. | AJ697663 | CAG26901.1 | | |
| 8F (Siat8F) | | | | | | | |
| β-galactosamide α-2,6- | | Pan troglodytes | 2.4.99.1 | AJ627624 | CAF29492.1 | | |
| sialyltransferase I | | | | | | | |
| (ST6Gal I; Siat1) | | | | | | | |
| β-galactosamide α-2,6- | | Pan troglodytes | n.d. | AJ627625 | CAF29493.1 | | |
| sialyltransferase II | | | | | | | |
| (ST6Gal II) | | | | | | | |
| GM3 synthase ST3Gal V | | Pan troglodytes | n.d. | AJ744807 | CAG32843.1 | | |
| (Siat9) | | | | | | | |
| S138L | | Rabbit fibroma | n.d. | NC_001266 | NP_052025 | | |
| | | virus Kasza | | | | | |
| α-2,3-sialyltransferase | | Rattus norvegicus | 2.4.99.6 | M97754 | AAA42146.1 | Q02734 | |
| ST3Gal III | | | | NM_031697 | NP_113885.1 | | |
| α-2,3-sialyltransferase | | Rattus norvegicus | n.d. | AJ626825 | CAF25183.1 | | |
| ST3Gal IV (Siat4c) | | | | | | | |
| α-2,3-sialyltransferase | | Rattus norvegicus | n.d. | AJ626743 | CAF25053.1 | | |
| ST3Gal VI | | | | | | | |
| α-2,6-sialyltransferase | | Rattus norvegicus | 2.4.99.— | X76988 | CAA54293.1 | Q11205 | |
| ST3Gal II | | | | NM_031695 | NP_113883.1 | | |
| α-2,6-sialyltransferase | | Rattus norvegicus | 2.4.99.1 | M18769 | AAA41196.1 | P13721 | |
| ST6Gal I | | | | M83143 | AAB07233.1 | | |
| α-2,6-sialyltransferase | | Rattus norvegicus | n.d. | AJ634458 | CAG25684.1 | | |
| ST6GalNAc I (Siat7A) | | | | | | | |
| α-2,6-sialyltransferase | | Rattus norvegicus | n.d. | AJ634457 | CAG25679.1 | | |
| ST6GalNAc II (Siat7B) | | | | | | | |
| α-2,6-sialyltransferase | | Rattus norvegicus | 2.4.99.— | L29554 | AAC42086.1 | Q64686 | |
| ST6GalNAc III | | | | BC072501 | AAH72501.1 | | |
| | | | | NM_019123 | NP_061996.1 | | |
| α-2,6-sialyltransferase | | Rattus norvegicus | n.d. | AJ646871 | CAG26700.1 | | |
| ST6GalNAc IV (Siat7D) | | | | | | | |
| (fragment) | | | | | | | |
| α-2,6-sialyltransferase | | Rattus norvegicus | n.d. | AJ646872 | CAG26701.1 | | |
| ST6GalNAc V (Siat7E) | | | | | | | |
| α-2,6-sialyltransferase | | Rattus norvegicus | n.d. | AJ646881 | CAG26710.1 | | |
| ST6GalNAc VI (Siat7F) | | | | | | | |
| (fragment) | | | | | | | |
| α-2,8-sialyltransferase | | Rattus norvegicus | 2.4.99.— | U53883 | AAC27541.1 | P70554 | |
| (GD3 synthase) ST8Sia I | | | | D45255 | BAA08213.1 | P97713 | |
| α-2,8-sialyltransferase | | Rattus norvegicus | n.d. | AJ699422 | CAG27884.1 | | |
| (SIAT8E) | | | | | | | |
| α-2,8-sialyltransferase | | Rattus norvegicus | n.d. | AJ699423 | CAG27885.1 | | |
| (SIAT8F) | | | | | | | |
| α-2,8-sialyltransferase | | Rattus norvegicus | 2.4.99.— | L13445 | AAA42147.1 | Q07977 | |
| ST8Sia II | | | | NM_057156 | NP_476497.1 | Q64688 | |
| α-2,8-sialyltransferase | | Rattus norvegicus | 2.4.99.— | U55938 | AAB50061.1 | P97877 | |
| ST8Sia III | | | | NM_013029 | NP_037161.1 | | |
| α-2,8-sialyltransferase | | Rattus norvegicus | 2.4.99.— | U90215 | AAB49989.1 | O08563 | |
| ST8Sia IV | | | | | | | |
| β-galactosamide α-2,6- | | Rattus norvegicus | n.d. | AJ627626 | CAF29494.1 | | |
| sialyltransferase II | | | | | | | |
| (ST6Gal II) | | | | | | | |
| GM3 synthase ST3Gal V | | Rattus norvegicus | n.d. | AB018049 | BAA33492.1 | O88830 | |
| | | | | NM_031337 | NP_112627.1 | | |
| sialyltransferase ST3Gal- | | Rattus norvegicus | n.d. | AJ748840 | CAG44449.1 | | |
| I (Siat4A) | | | | | | | |
| α-2,3-sialyltransferase | | Silurana tropicalis | n.d. | AJ585763 | CAE51387.1 | | |
| (St3Gal-II) | | | | | | | |
| α-2,6-sialyltransferase | | Silurana tropicalis | n.d. | AJ620650 | CAF05849.1 | | |
| (Siat7b) | | | | | | | |
| α-2,6-sialyltransferase | | Strongylocentrotus | n.d. | AJ699425 | CAG27887.1 | | |
| (St6galnac) | | purpuratus | | | | | |
| α-2,3-sialyltransferase | | Sus scrofa | n.d. | AJ585765 | CAE51389.1 | | |
| (ST3GAL-III) | | | | | | | |
| α-2,3-sialyltransferase | | Sus scrofa | n.d. | AJ584674 | CAE48299.1 | | |
| (ST3GAL-IV) | | | | | | | |
| α-2,3-sialyltransferase | | Sus scrofa | 2.4.99.4 | M97753 | AAA31125.1 | Q02745 | |
| ST3Gal I | | | | | | | |
| α-2,6-sialyltransferase | | Sus scrofa | 2.4.99.1 | AF136746 | AAD33059.1 | Q9XSG8 | |
| (fragment) ST6Gal I | | | | | | | |
| β-galactosamide α-2,6- | | Sus scrofa | n.d. | AJ620948 | CAF06585.2 | | |
| sialyltransferase | | | | | | | |
| (ST6GalNAc-V) | | | | | | | |
| sialyltransferase | | Sus scrofa | n.d. | AF041031 | AAC15633.1 | O62717 | |
| (fragment) ST6Gal I | | | | | | | |
| ST6GALNAC-V | | Sus scrofa | n.d. | AJ620948 | CAF06585.1 | | |
| α-2,3-sialyltransferase | | Takifugu rubripes | n.d. | AJ744805 | CAG32841.1 | | |
| (Siat5-r) | | | | | | | |
| α-2,3-sialyltransferase | | Takifugu rubripes | n.d. | AJ626816 | CAF25174.1 | | |
| ST3Gal I (Siat4) | | | | | | | |
| α-2,3-sialyltransferase | | Takifugu rubripes | n.d. | AJ626817 | CAF25175.1 | | |
| ST3Gal II (Siat5) | | | | | | | |
| (fragment) | | | | | | | |
| α-2,3-sialyltransferase | | Takifugu rubripes | n.d. | AJ626818 | CAF25176.1 | | |
| ST3Gal III (Siat6) | | | | | | | |
| α-2,6-sialyltransferase | | Takifugu rubripes | n.d. | AJ744800 | CAG32836.1 | | |
| ST6Gal I (Siat1) | | | | | | | |
| α-2,6-sialyltransferase | | Takifugu rubripes | n.d. | AJ634460 | CAG25681.1 | | |
| ST6GalNAc II (Siat7B) | | | | | | | |
| α-2,6-sialyltransferase | | Takifugu rubripes | n.d. | AJ634461 | CAG25682.1 | | |
| ST6GalNAc II B (Siat7B- | | | | | | | |
| related) | | | | | | | |
| α-2,6-sialyltransferase | | Takifugu rubripes | n.d. | AJ634456 | CAG25678.1 | | |
| ST6GalNAc III (Siat7C) | | | | | | | |
| (fragment) | | | | | | | |
| α-2,6-sialyltransferase | | Takifugu rubripes | 2.4.99.3 | Y17466 | CAB44338.1 | Q9W6U6 | |
| ST6GalNAc IV (siat7D) | | | | AJ646869 | CAG26698.1 | | |
| (fragment) | | | | | | | |
| α-2,6-sialyltransferase | | Takifugu rubripes | n.d. | AJ646873 | CAG26702.1 | | |
| ST6GalNAc V (Siat7E) | | | | | | | |
| (fragment) | | | | | | | |
| α-2,6-sialyltransferase | | Takifugu rubripes | n.d. | AJ646880 | CAG26709.1 | | |
| ST6GalNAc VI (Siat7F) | | | | | | | |
| (fragment) | | | | | | | |
| α-2,8-sialyltransferase | | Takifugu rubripes | n.d. | AJ715534 | CAG29373.1 | | |
| ST8Sia I (Siat 8A) | | | | | | | |
| (fragment) | | | | | | | |
| α-2,8-sialyltransferase | | Takifugu rubripes | n.d. | AJ715538 | CAG29377.1 | | |
| ST8Sia II (Siat 8B) | | | | | | | |
| (fragment) | | | | | | | |
| α-2,8-sialyltransferase | | Takifugu rubripes | n.d. | AJ715541 | CAG29380.1 | | |
| ST8Sia III (Siat 8C) | | | | | | | |
| (fragment) | | | | | | | |
| α-2,8-sialyltransferase | | Takifugu rubripes | n.d. | AJ715542 | CAG29381.1 | | |
| ST8Sia IIIr (Siat 8Cr) | | | | | | | |
| α-2,8-sialyltransferase | | Takifugu rubripes | n.d. | AJ715547 | CAG29386.1 | | |
| ST8Sia V (Siat 8E) | | | | | | | |
| (fragment) | | | | | | | |
| α-2,8-sialyltransferase | | Takifugu rubripes | n.d. | AJ715549 | CAG29388.1 | | |
| ST8Sia VI (Siat 8F) | | | | | | | |
| (fragment) | | | | | | | |
| α-2,8-sialyltransferase | | Takifugu rubripes | n.d. | AJ715550 | CAG29389.1 | | |
| ST8Sia VIr (Siat 8Fr) | | | | | | | |
| α-2,3-sialyltransferase | | Tetraodon | n.d. | AJ744806 | CAG32842.1 | | |
| (Siat5-r) | | nigroviridis | | | | | |
| α-2,3-sialyltransferase | | Tetraodon | n.d. | AJ744802 | CAG32838.1 | | |
| ST3Gal I (Siat4) | | nigroviridis | | | | | |
| α-2,3-sialyltransferase | | Tetraodon | n.d. | AJ626822 | CAF25180.1 | | |
| ST3Gal III (Siat6) | | nigroviridis | | | | | |
| α-2,6-sialyltransferase | | Tetraodon | n.d. | AJ634462 | CAG25683.1 | | |
| ST6GalNAc II (Siat7B) | | nigroviridis | | | | | |
| α-2,6-sialyltransferase | | Tetraodon | n.d. | AJ646879 | CAG26708.1 | | |
| ST6GalNAc V (Siat7E) | | nigroviridis | | | | | |
| (fragment) | | | | | | | |
| α-2,8-sialyltransferase | | Tetraodon | n.d. | AJ715536 | CAG29375.1 | | |
| ST8Sia I (Siat 8A) | | nigroviridis | | | | | |
| (fragment) | | | | | | | |
| α-2,8-sialyltransferase | | Tetraodon | n.d. | AJ715537 | CAG29376.1 | | |
| ST8Sia II (Siat 8B) | | nigroviridis | | | | | |
| (fragment) | | | | | | | |
| α-2,8-sialyltransferase | | Tetraodon | n.d. | AJ715539 | CAG29378.1 | | |
| ST8Sia III (Siat 8C) | | nigroviridis | | | | | |
| (fragment) | | | | | | | |
| α-2,8-sialyltransferase | | Tetraodon | n.d. | AJ715540 | CAG29379.1 | | |
| ST8Sia IIIr (Siat 8Cr) | | nigroviridis | | | | | |
| (fragment) | | | | | | | |
| α-2,8-sialyltransferase | | Tetraodon | n.d. | AJ715548 | CAG29387.1 | | |
| ST8Sia V (Siat 8E) | | nigroviridis | | | | | |
| (fragment) | | | | | | | |
| α-2,3-sialyltransferase | | Xenopus laevis | n.d. | AJ585762 | CAE51386.1 | | |
| (St3Gal-II) | | | | | | | |
| α-2,3-sialyltransferase | | Xenopus laevis | n.d. | AJ585766 | CAE51390.1 | | |
| (St3Gal-VI) | | | | | | | |
| α-2,3-sialyltransferase | | Xenopus laevis | n.d. | AJ585764 | CAE51388.1 | | |
| St3Gal-III (Siat6) | | | | AJ626823 | CAF25181.1 | | |
| α-2,8- | | Xenopus laevis | 2.4.99.— | AB007468 | BAA32617.1 | O93234 | |
| polysialyltransferase | | | | | | | |
| α-2,8-sialyltransferase | | Xenopus laevis | n.d. | AY272056 | AAQ16162.1 | | |
| ST8Siα-I (Siat8A; GD3 | | | | AY272057 | AAQ16163.1 | | |
| synthase) | | | | AJ704562 | CAG28695.1 | | |
| Unknown (protein for | | Xenopus laevis | n.d. | BC068760 | AAH68760.1 | | |
| MGC: 81265) | | | | | | | |
| α-2,3-sialyltransferase | | Xenopus tropicalis | n.d. | AJ626744 | CAF25054.1 | | |
| (3Gal-VI) | | | | | | | |
| α-2,3-sialyltransferase | | Xenopus tropicalis | n.d. | AJ622908 | CAF22058.1 | | |
| (Siat4c) | | | | | | | |
| α-2,6-sialyltransferase | | Xenopus tropicalis | n.d. | AJ646878 | CAG26707.1 | | |
| ST6GalNAc V (Siat7E) | | | | | | | |
| (fragment) | | | | | | | |
| α-2,8-sialyltransferase | | Xenopus tropicalis | n.d. | AJ715544 | CAG29383.1 | | |
| ST8Sia III (Siat 8C) | | | | | | | |
| (fragment) | | | | | | | |
| β-galactosamide α-2,6- | | Xenopus tropicalis | n.d. | AJ627628 | CAF29496.1 | | |
| sialyltransferase II | | | | | | | |
| (ST6Gal II) | | | | | | | |
| sialytransferase St8Sial | | Xenopus tropicalis | n.d. | AY652775 | AAT67042 | | |
| poly-α-2,8-sialosyl sialyltransferase | | Escherichia coli K1 | 2.4.—.— | M76370 | AAA24213.1 | Q57269 | |
| (NeuS) | | | | X60598 | CAA43053.1 | | |
| polysialyltransferase | | Escherichia coli K92 | 2.4.—.— | M88479 | AAA24215.1 | Q47404 | |
| α-2,8 polysialyltransferase SiaD | | Neisseria meningitidis | 2.4.—.— | M95053 | AAA20478.1 | Q51281 | |
| | | B1940 | | X78068 | CAA54985.1 | Q51145 | |
| SynE | | Neisseria meningitidis | n.d. | U75650 | AAB53842.1 | O06435 | |
| | | FAM18 | | | | | |
| polysialyltransferase (SiaD)(fragment) | | Neisseria meningitidis | n.d. | AY234192 | AAO85290.1 | | |
| | | M1019 | | | | | |
| SiaD (fragment) | | Neisseria meningitidis | n.d. | AY281046 | AAP34769.1 | | |
| | | M209 | | | | | |
| SiaD (fragment) | | Neisseria meningitidis | nd. | AY281044 | AAP34767.1 | | |
| | | M3045 | | | | | |
| polysialyltransferase (SiaD)(fragment) | | Neisseria meningitidis | n.d. | AY234191 | AAO85289.1 | | |
| | | M3315 | | | | | |
| SiaD (fragment) | | Neisseria meningitidis | n.d. | AY281047 | AAP34770.1 | | |
| | | M3515 | | | | | |
| polysialyltransferase (SiaD)(fragment) | | Neisseria meningitidis | n.d. | AY234190 | AAO85288.1 | | |
| | | M4211 | | | | | |
| SiaD (fragment) | | Neisseria meningitidis | n.d. | AY281048 | AAP34771.1 | | |
| | | M4642 | | | | | |
| polysialyltransferase (SiaD)(fragment) | | Neisseria meningitidis | n.d. | AY234193 | AAO85291.1 | | |
| | | M5177 | | | | | |
| SiaD | | Neisseria meningitidis | n.d. | AY281043 | AAP34766.1 | | |
| | | M5178 | | | | | |
| SiaD (fragment) | | Neisseria meningitidis | n.d. | AY281045 | AAP34768.1 | | |
| | | M980 | | | | | |
| NMB0067 | | Neisseria meningitidis | n.d. | NC_003112 | NP_273131 | | |
| | | MC58 | | | | | |
| Lst | | Aeromonas punctata | n.d. | AF126256 | AAS66624.1 | | |
| | | Sch3 | | | | | |
| ORF2 | | Haemophilus influenzae | n.d. | M94855 | AAA24979.1 | | |
| | | A2 | | | | | |
| HI1699 | | Haemophilus influenzae | n.d. | U32842 | AAC23345.1 | Q48211 | |
| | | Rd | | NC_000907 | NP_439841.1 | | |
| α-2,3-sialyltransferase | | Neisseria gonorrhoeae | 2.4.99.4 | U60664 | AAC44539.1 | P72074 | |
| | | F62 | | | AAE67205.1 | | |
| α-2,3-sialyltransferase | | Neisseria meningitidis | 2.4.99.4 | U60662 | AAC44544.2 | | |
| | | 126E, NRCC 4010 | | | | | |
| α-2,3-sialyltransferase | | Neisseria meningitidis | 2.4.99.4 | U60661 | AAC44543.1 | | |
| | | 406Y, NRCC 4030 | | | | | |
| α-2,3-sialyltransferase | | Neisseria meningitidis | 2.4.99.4 | U60660 | AAC44541.1 | P72097 | |
| (NMB0922) | | MC58 | | AE002443 | AAF41330.1 | | |
| | | | | NC_003112 | NP_273962.1 | | |
| NMA1118 | | Neisseria meningitidis | n.d. | AL162755 | CAB84380.1 | Q9JUV5 | |
| | | Z2491 | | NC_003116 | NP_283887.1 | | |
| PM0508 | | Pasteurella multocida | n.d. | AE006086 | AAK02592.1 | Q9CNC4 | |
| | | PM70 | | NC_002663 | NP_245445.1 | | |
| WaaH | | Salmonella enterica | n.d. | AF519787 | AAM82550.1 | Q8KS93 | |
| | | SARB25 | | | | | |
| WaaH | | Salmonella enterica | n.d. | AF519788 | AAM82551.1 | Q8KS92 | |
| | | SARB3 | | | | | |
| WaaH | | Salmonella enterica | n.d. | AF519789 | AAM82552.1 | | |
| | | SARB39 | | | | | |
| WaaH | | Salmonella enterica | n.d. | AF519790 | AAM82553.1 | | |
| | | SARB53 | | | | | |
| WaaH | | Salmonella enterica | n.d. | AF519791 | AAM82554.1 | Q8KS91 | |
| | | SARB57 | | | | | |
| WaaH | | Salmonella enterica | n.d. | AF519793 | AAM82556.1 | Q8KS89 | |
| | | SARB71 | | | | | |
| WaaH | | Salmonella enterica | n.d. | AF519792 | AAM82555.1 | Q8KS90 | |
| | | SARB8 | | | | | |
| WaaH | | Salmonella enterica | n.d. | AF519779 | AAM88840.1 | Q8KS99 | |
| | | SARC10V | | | | | |
| WaaH (fragment) | | Salmonella enterica | n.d. | AF519781 | AAM88842.1 | | |
| | | SARC12 | | | | | |
| WaaH (fragment) | | Salmonella enterica | n.d. | AF519782 | AAM88843.1 | Q8KS98 | |
| | | SARC13I | | | | | |
| WaaH (fragment) | | Salmonella enterica | n.d. | AF519783 | AAM88844.1 | Q8KS97 | |
| | | SARC14I | | | | | |
| WaaH | | Salmonella enterica | n.d. | AF519784 | AAM88845.1 | Q8KS96 | |
| | | SARC15II | | | | | |
| WaaH | | Salmonella enterica | n.d. | AF519785 | AAM88846.1 | Q8KS95 | |
| | | SARC16II | | | | | |
| WaaH (fragment) | | Salmonella enterica | n.d. | AF519772 | AAM88834.1 | Q8KSA4 | |
| | | SARC3I | | | | | |
| WaaH (fragment) | | Salmonella enterica | n.d. | AF519773 | AAM88835.1 | Q8KSA3 | |
| | | SARC4I | | | | | |
| WaaH | | Salmonella enterica | n.d. | AF519774 | AAM88836.1 | | |
| | | SARC5IIa | | | | | |
| WaaH | | Salmonella enterica | n.d. | AF519775 | AAM88837.1 | Q8KSA2 | |
| | | SARC6IIa | | | | | |
| WaaH | | Salmonella enterica | n.d. | AF519777 | AAM88838.1 | Q8KSA1 | |
| | | SARC8 | | | | | |
| WaaH | | Salmonella enterica | n.d. | AF519778 | AAM88839.1 | Q8KSA0 | |
| | | SARC9V | | | | | |
| UDP-glucose: α-1,2- | | Salmonella enterica | 2.4.1.— | AF511116 | AAM48166.1 | | |
| glucosyltransferase (WaaH) | | subsp. arizonae SARC5 | | | | | |
| bifunctional α-2,3/-2,8- | | Campylobacter jejuni | n.d. | AF401529 | AAL06004.1 | Q930Z5 | |
| sialyltransferase (Cst-II) | | ATCC 43449 | | | | | |
| Cst | | Campylobacter jejuni | n.d. | AF305571 | AAL09368.1 | | |
| | | 81-176 | | | | | |
| α-2,3-sialyltransferase (Cst- | | Campylobacter jejuni | 2.4.99.— | AY044156 | AAK73183.1 | | |
| III) | | ATCC 43429 | | | | | |
| α-2,3-sialyltransferase (Cst- | | Campylobacter jejuni | 2.4.99.— | AF400047 | AAK85419.1 | | |
| III) | | ATCC 43430 | | | | | |
| α-2,3-sialyltransferase (Cst- | | Campylobacter jejuni | 2.4.99.— | AF215659 | AAG43979.1 | Q9F0M9 | |
| II) | | ATCC 43432 | | | | | |
| α-2,3/8-sialyltransferase | | Campylobacter jejuni | n.d. | AF400048 | AAK91725.1 | Q93MQ0 | |
| (CstII) | | ATCC 43438 | | | | | |
| α-2,3-sialyltransferase cst-II | | Campylobacter jejuni | 2.4.99.— | AF167344 | AAF34137.1 | | |
| | | ATCC 43446 | | | | | |
| α-2,3-sialyltransferase (Cst- | | Campylobacter jejuni | 2.4.99.— | AF401528 | AAL05990.1 | Q93D05 | |
| II) | | ATCC 43456 | | | | | |
| α-2,3-/α-2,8-sialyltransferase | | Campylobacter jejuni | 2.4.99.— | AY044868 | AAK96001.1 | Q938X6 | |
| (CstII) | | ATCC 43460 | | | | | |
| α-2,3/8-sialyltransferase | | Campylobacter jejuni | n.d. | AF216647 | AAL36462.1 | | |
| (Cst-II) | | ATCC 700297 | | | | | |
| ORF | | Campylobacter jejuni | n.d. | AY422197 | AAR82875.1 | | |
| | | GB11 | | | | | |
| α-2,3-sialyltransferase cstIII | | Campylobacter jejuni | 24.99.— | AF195055 | AAG29922.1 | | |
| | | MSC57360 | | | | | |
| α-2,3-sialyltransferase cstIII | | Campylobacter jejuni | 2.4.99.— | AL139077 | CAB73395.1 | Q9PNF4 | |
| Cj1140 | | NCTC 11168 | | NC_002163 | NP_282288.1 | | |
| α-2,3/α-2,8-sialyltransferase | | Campylobacter jejuni | n.d. | — | AAO96669.1 | | |
| II (cstII) | | O: 10 | | AX934427 | CAF04167.1 | | |
| α-2,3/α-2,8-sialyltransferase | | Campylobacter jejuni | n.d. | AX934431 | CAF04169.1 | | |
| II (CstII) | | O: 19 | | | | | |
| α-2,3/α-2,8-sialyltransferase | | Campylobacter jejuni | n.d. | AX934436 | CAF04171.1 | | |
| II (CstII) | | O: 36 | | | | | |
| α-2,3/α-2,8-sialyltransferase | | Campylobacter jeluni | n.d. | AX934434 | CAF04170.1 | | |
| II (CstII) | | O: 4 | | | | | |
| α-2,3/α-2,8-sialyltransferase | | Campylobacter jejuni | n.d. | — | AAO96670.1 | | |
| II (CstII) | | O: 41 | | — | AAT17967.1 | | |
| | | | | AX934429 | CAF04168.1 | | |
| α-2,3-sialyltransferase cst-I | | Campylobacter jejuni | 2.4.99.— | AF130466 | AAF13495.1 | Q9RGF1 | |
| | | OH4384 | | — | AAS36261.1 | | |
| bifunctional α-2,3/-2,8- | | Campylobacter jejuni | 2.4.99.— | AF130984 | AAF31771.1 | | 1RO7 C |
| sialyltransferase (Cst-Il) | | OH4384 | | AX934425 | CAF04166.1 | | 1RO8 A |
| HI0352 (fragment) | | Haemophilus | n.d. | U32720 | AAC22013.1 | P24324 | |
| | | influenzae Rd | | X57315 | CAA40567.1 | | |
| | | | | NC_000907 | NP_438516.1 | | |
| PM1174 | | Pasteurella multocida | n.d. | AE006157 | AAK03258.1 | Q9CLP3 | |
| | | PM70 | | NC_002663 | NP_246111.1 | | |
| Sequence 10 from patent US | | Unknown. | n.d. | — | AAO96672.1 | | |
| 6503744 | | | | | | | |
| Sequence 10 from patent US | | Unknown. | n.d. | — | AAT17969.1 | | |
| 6699705 | | | | | | | |
| Sequence 12 from patent US | | Unknown. | n.d. | — | AAT17970.1 | | |
| 6699705 | | | | | | | |
| Sequence 2 from patent US | | Unknown. | n.d. | — | AAT23232.1 | | |
| 6709834 | | | | | | | |
| Sequence 3 from patent US | | Unknown. | n.d. | — | AAO96668.1 | | |
| 6503744 | | | | | | | |
| Sequence 3 from patent US | | Unknown. | n.d. | — | AAT17965.1 | | |
| 6699705 | | | | | | | |
| Sequence 34 from patent US | | Unknown. | nd. | — | AAO96684.1 | | |
| 6503744 | | | | | | | |
| Sequence 35 from patent US | | Unknown. | n.d. | — | AAO96685.1 | | |
| 6503744 (fragment) | | | | — | AAS36262.1 | | |
| Sequence 48 from patent US | | Unknown. | n.d. | — | AAT17988.1 | | |
| 6699705 | | | | | | | |
| Sequence 5 from patent US | | Unknown. | n.d. | — | AAT17966.1 | | |
| 6699705 | | | | | | | |
| Sequence 9 from patent US | | Unknown. | n.d. | — | AAO96671.1 | | |
| 6503744 | | | | | | | |

In addition to the sialyltransferases listed in Tables 3 and 4, the invention also includes use of the following sialyltransferases: protein encoded by the siaA protein of Haemophilus influenzae, accession number AAL38659; an α2,6-sialyltransferase gene from Photobacterium damsela, accession number BAA25316; protein from Pasteurella multocida, accession number NP_{—}245125; and protein from Haemophilus ducreyi, accession number NP_{—}872679.

2. Fucosyltransferases

In some embodiments, the glycosyltransferase is a fucosyltransferase. A number of fucosyltransferases are known to those of skill in the art. Briefly, fucosyltransferases include any of those enzymes which transfer L-fucose from GDP-fucose to a hydroxy position of an acceptor sugar. In some embodiments, the acceptor sugar is, for example, the GlcNAc in a Galβ(1→4)GlcNAcβ-group in an oligosaccharide glycoside.

Bacterial fucosyltransferases are known and are useful in the present invention. An α1,3-fucosyltransferase gene from Helicobacter pylori has also been characterized (Martin et al. (1997) J. Biol. Chem. 272: 21349-21356), as have α1,3-4-fucosyltransferase gene and α1,2-fucosyltransferase gene. For example fucosyltransferases from Helicobacter pylori are disclosed in U.S. Pat. Nos. 6,534,298 and 6,238,894; WO2004009838, published Jan. 29, 2004; U.S. Ser. No. 10/764,212, filed Jan. 22, 2004; each of which are herein incorporated by reference for all purposes.

Suitable eukaryotic fucosyltransferases for this reaction include the Galβ(1→3,4)GlcNAcβ1-α(1→3,4)fucosyltransferase (FTIII E.C. No. 2.4.1.65), which was first characterized from human milk (see, Palcic, et al., Carbohydrate Res. 190:1-11 (1989); Prieels, et al., J. Biol. Chem. 256:10456-10463 (1981); and Nunez, et al., Can. J. Chem. 59:2086-2095 (1981)) and the Galβ(1→4)GlcNAcβ-α(1→3)fucosyltransferases (FTIV, FTV, FTVI, and FTVII, E.C. No. 2.4.1.65) which are found in human serum. A recombinant form of the Galβ(1→3,4) GlcNAcβ-α(1→3,4)fucosyltransferase has also been characterized (see, Dumas, et al., Bioorg. Med. Letters 1:425-428 (1991) and Kukowska-Latallo, et al., Genes and Development 4:1288-1303 (1990)). Other exemplary fucosyltransferases include, for example, α1,2 fucosyltransferase (E.C. No. 2.4.1.69). Enzymatic fucosylation can be carried out by the methods described in Mollicone, et al., Eur. J. Biochem. 191:169-176 (1990) or U.S. Pat. No. 5,374,655. In some embodiments, cells that are used to produce a fucosyltransferase will also include an enzymatic system for synthesizing GDP-fucose. Human fucosyltransferases include α(1→3,3, or 4)fucosyltransferases and can be used in the methods of the invention. Eukaryotic fucosyltransferases generally comprise different functional domains, e.g., a cytoplasmic domain, a signal-anchor domain, a stem region and a catalytic domain. In preferred embodiments, the catalytic domain of a eukaryotic fucosyltransferase is expressed in a host cell.

3. Galactosyltransferases

In another group of embodiments, a galactosyltransferase is used in the invention. When a galactosyltransferase is used, the cell that contains the exogenous galactosyltransferase gene will, in some embodiments, also contain an enzymatic system for synthesizing UDP-Gal.

In some embodiments, galactosyltransferases are used to make a lactose disaccharide from glucose. In one embodiment a β(1,4) galactosyltransferase from Neisseria meningitides/gonorrhoeae is used (lgtB). See, e.g., Park et al., J Biochem Mol Biol. 35:330-6 (2002), which is herein incorporated by reference for all purposes. In another embodiment an α(1,4) galactosyltransferase from Neisseria is used, see, e.g., lgtC accession number U14554. Also suitable for use in the methods and recombinant cells of the invention are β(1,4) galactosyltransferases, which include, for example, EC 2.4.1.90 (LacNAc synthetase) and EC 2.4.1.22 (lactose synthetase) (bovine (D'Agostaro et al (1989) Eur. J. Biochem. 183:211-217), human (Masri et al. (1988) Biochem. Biophys. Res. Commun. 157:657-663), murine (Nakazawa et al. (1988) J. Biochem. 104:165-168), as well as E.C. 2.4.1.38 and the ceramide galactosyltransferase (EC 2.4.1.45, Stahl et al. (1994) J. Neurosci. Res. 38:234-242). Other suitable galactosyltransferases include, for example, α1,2 galactosyltransferases (from e.g., Schizosaccharomyces pombe, Chapell et al (1994) Mol. Biol. Cell 5:519-528).

Other bacterial galactosyltransferases useful in the invention include e.g., β1,3-galactosyltransferases from C. jejuni, disclosed in U.S. Pat. No. 6,699,705, issued Mar. 2, 2004, herein incorporated by reference for all purposes.

Other galactosyltransferases include α(1,3) galactosyltransferases (E.C. No. 2.4.1.151, see, e.g., Dabkowski et al., Transplant Proc. 25:2921 (1993) and Yamamoto et al. Nature 345:229-233 (1990), bovine (GenBank j04989, Joziasse et al. (1989) J. Biol. Chem. 264:14290-14297), murine (GenBank m26925; Larsen et al. (1989) Proc. Nat'l. Acad. Sci. USA 86:8227-8231), porcine (GenBank L36152; Strahan et al (1995) Immunogenetics 41:101-105)). Another suitable α1,3 galactosyltransferase is that which is involved in synthesis of the blood group B antigen (EC 2.4.1.37, Yamamoto et al. (1990) J. Biol. Chem. 265:1146-1151 (human)).

4. Other Glycosyltransferases

Other glycosyltransferases that can be contained by the recombinant host cells of the invention have been described in detail, as for the sialyltransferases, galactosyltransferases, and fucosyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, α(1,3) N-acetylgalactosaminyltransferase, β(1,4) N-acetylgalactosaminyltransferases (Nagata et al. J. Biol. Chem. 267:12082-12089 (1992) and Smith et al. J. Biol. Chem. 269:15162 (1994)) and polypeptide N-acetylgalactosaminyltransferase (Homa et al. J. Biol. Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J. 285:985 (1992), and hyaluronan synthase. Suitable mannosyltransferases include ((1,2) mannosyltransferase, α(1,3) mannosyltransferase, β(1,4) mannosyltransferase, Dol-P-Man synthase, OCh1, and Pmt1.

Prokaryotic glycosyltransferases are also useful in the recombinant cells and reaction mixtures of the invention. Such glycosyltransferases include enzymes involved in synthesis of lipooligosaccharides (LOS), which are produced by many gram negative bacteria. The LOS typically have terminal glycan sequences that mimic glycoconjugates found on the surface of human epithelial cells or in host secretions (Preston et al. (1996) Critical Reviews in Microbiology 23(3): 139-180). Such enzymes include, but are not limited to, the proteins of the rfa operons of species such as E. coli and Salmonella typhimurium, which include a β1,6 galactosyltransferase and a β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an β1,2-glucosyltransferase (rfaJ) (Swiss-Prot Accession No. P27129 (E. coli) and Swiss-Prot Accession No. P19817 (S. typhimurium)), and an β1,2-N-acetylglucosaminyltransferase (rfaK) (EMBL Accession No. U00039 (E. coli). Prokaryotic glycosyltransferases from the LOS locus of C. jejuni can also be used in the invention are disclosed in U.S. Pat. No. 6,699,705, issued Mar. 2, 2004, herein incorporated by reference for all purposes; and include e.g., β1,4-GalNAc transferases, such as cgtA. Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rh1 operon of Pseudomonas aeruginosa.

Also suitable for use in the cells of the invention are glycosyltransferases that are involved in producing structures containing lacto-N-neotetraose, D-galactosyl-β-1,4-N-acetyl-D-glucosaminyl-P-1,3-D-galactosyl-β-1,4-D-glucose, and the P^{k} blood group trisaccharide sequence, D-galactosyl-β-1,4-D-galactosyl-β-1,4-D-glucose, which have been identified in the LOS of the mucosal pathogens Neisseria gonnorhoeae and N. meningitidis (Scholten et al. (1994) J. Med. Microbiol. 41: 236-243). The genes from N. meningitidis and N. gonorrhoeae that encode the glycosyltransferases involved in the biosynthesis of these structures have been identified from N. meningitidis immunotypes L3 and L1 (Jennings et al. (1995) Mol. Microbiol. 18: 729-740) and the N. gonorrhoeae mutant F62 (Gotshlich (1994) J. Exp. Med. 180: 2181-2190). In N. meningitidis, a locus consisting of three genes, lgtA, lgtB and lgE, encodes the glycosyltransferase enzymes required for addition of the last three of the sugars in the lacto-N-neotetraose chain (Wakarchuk et al. (1996) J. Biol. Chem. 271: 19166-73). Recently the enzymatic activity of the lgtB and lgtA gene product was demonstrated, providing the first direct evidence for their proposed glycosyltransferase function (Wakarchuk et al. (1996) J. Biol. Chem. 271 (45): 28271-276). In N. gonorrhoeae, there are two additional genes, lgtD which adds β-D-GalNAc to the 3 position of the terminal galactose of the lacto-N-neotetraose structure and lgtC which adds a terminal α-D-Gal to the 4 position of the lactose element of a truncated LOS, thus creating the P^{k} blood group antigen structure (Gotshlich (1994), supra.). In N. meningitidis, a separate immunotype L1 also expresses the P^{k} blood group antigen and has been shown to carry an lgtC gene (Jennings et al. (1995), supra.). Neisseria glycosyltransferases and associated genes are also described in U.S. Pat. No. 5,545,553 (Gotschlich).

C. Fusion Proteins Comprising a Glycosyltransferases and an Accessory Enzyme

In some embodiments, the recombinant cells of the invention express fusion proteins that have more than one enzymatic activity that is involved in synthesis of a desired sialylated oligosaccharide. The fusion polypeptides can be composed of, for example, a catalytic domain of a sialyltransferase that is joined to a catalytic domain of an accessory enzyme, e.g., CMP-sialic acid synthase. For example, a polynucleotide that encodes a sialyltransferase can be joined, in-frame, to a polynucleotide that encodes an enzyme involved in CMP-sialic acid synthesis. The resulting fusion protein can then catalyze not only the synthesis of the activated sialic acid molecule, but also the transfer of the sialic acid moiety to the acceptor molecule. The fusion protein can be two or more sialic acid cycle enzymes linked into one expressable nucleotide sequence. The fusion sialyltransferase polypeptides of the present invention can be readily designed and manufactured utilizing various recombinant DNA techniques well known to those skilled in the art. Suitable fusion proteins are described in PCT Patent Application PCT/CA98/01180, which was published as WO99/31224 on Jun. 24, 1999 and which discloses CMP-sialic acid synthase from Neisseria fused with an α2,3-sialyltransferase from Neisseria. Those of skill will recognize that many other CMP-sialic acid synthase polypeptides and sialyltransferases can be fused for use in the invention. In some embodiments, a CMP-sialic acid synthase from Neisseria is fused to a sialyltransferase from C. jejuni. The C. jejuni sialyltransferase (Cst) can be a CstI, CstII, or CstIII enzyme. A full-length or truncated version of the C. jejuni sialyltransferase polypeptide can be used in the fusion sialyltransferase protein. In some embodiments, more that one fusion sialyltransferase polypeptide is expressed in the cell.

In some embodiments, the recombinant cells of the invention express fusion proteins that have more than one enzymatic activity that is involved in addition of at least one additional sugar residue, e.g., a non-sialic acid residue. These fusion polypeptides can be composed of, for example, a catalytic domain of a glycosyltransferase, e.g., not a sialyltransferase, that is joined to a catalytic domain of an accessory enzyme. The accessory enzyme catalytic domain can, for example, catalyze a step in the formation of a nucleotide sugar which is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressable nucleotide sequence. The polypeptides of the present invention can be readily designed and manufactured utilizing various recombinant DNA techniques well known to those skilled in the art. Suitable fusion proteins are described in PCT Patent Application PCT/CA98/01180, which was published as WO99/31224 on Jun. 24, 1999, which is herein incorporated by reference for all purposes. The disclosed fusion proteins include e.g., a UDP glucose epimerase fused in frame to a galactosyltransferase and a sialyltransferase fused in frame to a CMP-sialic acid synthase. Eukaryotic enzymes (e.g., galactosyl transferases) can also be sued in the fusion glycosyltransferases of the invention. (See, e.g., Chen et al., J. Biol. Chem. 275:31594-31600 (2000))

### V. Microorganisms for Use in the Present Invention

The recombinant cells of the invention are generally made by creating or otherwise obtaining a polynucleotide that encodes the particular enzyme(s) of interest, modifying the polynucleotide as desired, placing the polynucleotide in an expression cassette under the control of a promoter and other appropriate control signals, and introducing the expression cassette into a cell. More than one of the enzymes can be expressed in the same host cells using a variety of methods. For example, a single extrachromosomal vector can include multiple expression cassettes or more that one compatible extrachromosomal vector can be used maintain an expression cassette in a host cell. Expression cassettes can also be inserted into a host cell chromosome, using methods known to those of skill in the art. Those of skill will recognize that combinations of expression cassettes in extrachromosomal vectors and expression cassettes inserted into a host cell chromosome can also be used. Other modification of the host cell, described in detail below, can be performed to enhance production of the desired oligosaccharide.

The invention includes recombinant cells that can be constructed using methods known to those of skill in the art. The recombinant cells of the invention can contain a heterologous gene that encodes a glycosyltransferase and a heterologous gene that encodes an accessory enzyme involved in synthesis of a donor sugar. As an example, a recombinant cell can contain a sialyltransferase, or an exogenous CMP-sialic acid synthase, or an enzymatic system for synthesizing sialic acid that is wholly or in part exogenous, or some combination of the three.

In some embodiments, the recombinant cells of the invention can produce multiple nucleotide sugars or nucleotides, thus allowing the introduction of multiple glycosyltransferases or multiple glycosyltransferases with supporting cycle enzymes, respectively, to produce the target oligosaccharide. This allows the production of multiple glycosidic linkages in a product using a single organism. For example, if the organism produces both UDP-Gal and UDP-GlcNAc, then addition of a Gal transferase and a GlcNAc transferase would allow the production of two new glycosidic linkages from the same organism. As another example, if an organism produces elevated levels of UTP, then by adding genes that encode enzymes for the production of UDP-Gal and UDP-GlcNAc, as well as genes that encode a Gal-transferase and a GlcNAc transferase two new glycosidic linkages can be formed from a single organism. In these examples, if the transferases allow glycosidic polymerization, then long chain oligosaccharides and polysaccharides can be formed.

Typically, the polynucleotide that encodes the heterologous glycosyltransferase or the heterologous accessory enzyme, is placed under the control of a promoter that is functional in the desired host cell. An extremely wide variety of promoters are well known, and can be used to regulate expression of recombinant proteins, depending on the particular application. Ordinarily, the promoter selected depends upon the cell in which the promoter is to be active. Other expression control sequences such as ribosome binding sites, transcription termination sites and the like are also optionally included. Expression control sequences that are suitable for use in a particular host cell are often obtained by cloning a gene that is expressed in that cell.

The recombinant cells of the invention are generally microorganisms, such as, for example, yeast cells, bacterial cells, or fungal cells. Examples of suitable cells include, for example, Azotobacter sp. (e.g., A. vinelandii), Pseudomonas sp., Rhizobium sp., Erwinia sp., Bacillus sp., Streptomyces sp., Escherichia sp. (e.g., E. coli), and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus).

A promoter and other control signals can be derived from a gene that is under investigation, or can be a heterologous promoter or other signal that is obtained from a different gene, or from a different species. Where continuous expression of a gene is desired, one can use a “constitutive” promoter, which is generally active under most environmental conditions and states of development or cell differentiation.

Promoters for use in E. coli include the T7, trp, or lambda promoters. A ribosome binding site and preferably a transcription termination signal are also provided. For expression of heterologous proteins in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli. Methods of transforming prokaryotes other than E. coli are well known. For example, methods of transforming Bacillus species and promoters that can be used to express proteins are taught in U.S. Pat. No. 6,255,076 and U.S. Pat. No. 6,770,475, both of which are herein incorporated by reference for all purposes.

In yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4: 2093 2099 (1985)) and the tpiA promoter. An example of a suitable terminator is the ADH3 terminator (McKnight et al.).

In some embodiments, the polynucleotides are placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter. A particularly preferred inducible promoter for expression in prokaryotes is a dual promoter that includes a tac promoter component linked to a promoter component obtained from a gene or genes that encode enzymes involved in galactose metabolism (e.g., a promoter from a UDPgalactose 4-epimerase gene (galE)). The dual tac-gal promoter, which is described in U.S. Ser. No. 08/965,850, filed Nov. 7, 1997, provides a level of expression that is greater than that provided by either promoter alone. Expression vectors of the pcWin family, e.g., pcWin1, pcWin2, and pcWin2-MBP, can also be used in the present invention. See, e.g., U.S. 60/535,263; filed Jan. 9, 2004; which is herein incorporated by reference for all purposes.

Inducible promoters for other organisms are also well known to those of skill in the art. These include, for example, the arabinose promoter, the lacZ promoter, the metallothionein promoter, and the heat shock promoter, as well as many others.

A construct that includes a polynucleotide of interest operably linked to gene expression control signals that, when placed in an appropriate host cell, drive expression of the polynucleotide is termed an “expression cassette.” Expression cassettes that encode the glycosyltransferase and/or enzyme involved in nucleotide sugar synthesis are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in U.S. Ser. No. 08/965,850, filed Nov. 7, 1997. Other expression vectors are the pcWin vectors, disclosed in U.S. Ser. No. 60/535,263, filed Jan. 9, 2004, which is herein incorporated by reference for all purposes.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to construct the cells of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the target cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in culture medium, thus removing selective pressure and allowing the culture to become overgrown with cells that do not contain the vector.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable for constructing the recombinant cells of the invention are well known in the art. For cloning in bacteria, common vectors include pBR322 derived vectors such as pBLUESCRIPT™, and λ-phage derived vectors. In yeast, vectors include Yeast Integrating plasmids (e.g., YIp5) and Yeast Replicating plasmids (the YRp series plasmids) and pGPD-2.

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

In some embodiments, a single expression vector is constructed for expression of an enzymatic system for synthesizing sialic acid, a CMP-sialic acid synthase, and a sialyltransferase. The nucleic acids encoding the above proteins are cloned into an expression cassette, under the control of a single promoter. In an alternative embodiment, the CMP-sialic acid synthase, and the sialyltransferase are fused to form a single fusion protein. As an example of a single expression vector, nucleic acids encoding a GlcNAc epimerase, an N-acetyl neuraminic acid (NANA) condensing polypeptide, a fusion between a CMP-sialic acid synthase, and a sialyltransferase are cloned into single expression vector and expressed in bacteria for use in the methods of the invention.

In some embodiments, production of oligosaccharides is enhanced by manipulation of the host microorganism. For example, in E. coli, break down of sialic acid can be minimized by using a host strain that has diminished CMP-sialic acid synthase activity (NanA-). (In E. coli, CMP-sialic acid synthase appears to be a catabolic enzyme.) Diminishing the sialic acid degradative pathway in a host cell can be accomplished by disrupting the N-acetylneuraminate lyase gene (NanA, Accession number AE000402 region 70-963) or the N-acetylmannosamine kinase gene (NanK, see, e.g., Ringenberg et al., Mol Microbiol. 50:961-75 (2003)). As another example, Escherichia sp., including E. coli, can produce a membrane-bound polysialic acid. Mutant strains in which synthesis of the polysialic acid is disrupted accumulate CMP-sialic acid (Vimr and Troy (1985) J. Bact. 164: 854-860; Gonzalez-Clemente et al. (1990) Biol. Chem. 371: 1101-1106; Cho et al. (1994) Proc. Nat'l. Acad. Sci. USA 91: 11427-11431). Introduction of a sialyltransferase gene into these mutant strains results in a recombinant cell that is capable of producing large amounts of a sialylated product saccharide.

In some embodiments a heterologous pyrG gene is introduced into a host cell. pyrG genes encode CTP synthethase proteins. Expression of a heterologous pyrG protein increases CTP pools from UTP, leading to increases in the CMP-sialic acid pool. pyrG genes have been identified in a number of organisms, including e.g., E. coli, Weng et al., J. Biol. Chem. 261:5568-5574 (1986); accession number M12843.

If a fucosylated product is being synthesized, appropriate metabolic pathways can be manipulated. For example, the extracellular polysaccharide colanic acid is produced by E. coli, using GDP-fucose as a precursor. Accordingly, one can disrupt the activity of an enzyme involved in the conversion of GDP-fucose to colanic acid (e.g., GDP-Man 4,6-dehydratase; Stevenson et al. (1996) J. Bacteriol. 178: 4885-4893).

In some embodiments, the microorganisms are manipulated to enhance transport of an acceptor saccharide into the cell. For example, where lactose is the acceptor saccharide, E. coli cells that express or overexpress the LacY permease can be used. Also in E. coli, when lactose is the acceptor saccharide or an intermediate in synthesizing the sialylated product, lactose breakdown can be minimized by using host cells that are LacZ-.

In additional embodiments, the recombinant cells of the invention produce a nucleotide sugar at an elevated level compared to a wild-type cell, and/or a nucleotide sugar produced by the cell is diverted from, for example, production of a polysaccharide to production of a desired product saccharide. For example, Azobacter vinelandii and Pseudomonas aeruginosa produce relatively large amounts of GDP-Man, the majority of which is used in the synthesis of the polysaccharide alginate. By disrupting the ability of the cells to produce alginate, one can obtain cells that produce increased levels of GDP-Man. Alginate synthesis in Pseudomonas and Azobacter involves GDP-mannose dehydrogenase, which converts GDP-Man to GDP-mannuronic acid, which is a direct precursor of alginate (Tatnell et al. (1994) Microbiol. 140: 1745-1754; Tatnell et al. (1993) J. Gen. Microbiol. 139(Pt. 1): 119-127; Lloret et al. (1996) Mol. Microbiol. 21: 449-457). By introducing a mutation that disrupts GDP-Man dehydrogenase activity, for example, one can obtain a cell that produces a higher level of GDP-Man than a wild-type cell. If a gene that encodes a glycosyltransferase that uses GDP-Man as a substrate is introduced into the cell, the GDP-Man that is no longer used for alginate synthesis is diverted to the synthesis of a desired mannosylated oligosaccharide. Alternatively, one can introduce genes that encode one or more enzymes such as those described above that convert GDP-Man to a different activated sugar, such as GDP-Fuc. The resulting recombinant cells can then be used for producing a fucosylated oligosaccharide of interest.

Similarly, one can construct a recombinant cell in which UDP-GlcNAc utilization is diverted from synthesis of peptidoglycan to synthesis of a desired GlcNAc-containing oligosaccharide. In E. coli, for example, a series of six enzymes, which act sequentially, are involved in conversion of UDP-GlcNAc into precursors of peptidoglycans (Mengin-Lecreulx et al. (1983) J. Bact. 154: 1284-1290). By disrupting one of these enzymes, preferably the first-acting enzyme, and introducing a GlcNAc transferase into the cell, one can divert the large quantities of UDP-GlcNAc produced by the cell to production of a desired GlcNAc-containing oligosaccharide. Alternatively, introduction of a gene encoding UDP-GlcNAc 4′-epimerase can result in conversion of UDP-GlcNAc to UDP-GalNAc, which can then serve as a sugar donor for a UDP-GalNAc transferase, which is encoded by an exogenous gene that is also introduced into the cell.

Bacteria belonging to the genera Azorhizobium, Bradyrhizobium, Rhizobium, and Sinorhizobium can produce lipo-chitooligosaccharides (LCOs). In at least some of these genera, a fucosyltransferase is encoded that uses GDP-fucose as a donor for transfer of fucose to LCO precursors (Mergaert et al. (1997) FEBS Lett. 409: 312-316). Accordingly, by disrupting the activity of this fucosyltransferase, one can divert the GDP-fucose produced by the cells to other uses. For example, a different fucosyltransferase gene can be introduced into the cells, thus obtaining a recombinant cell that produces a desired fucosylated saccharide.

Other examples of organisms and associated nucleotide sugars that one can divert to production of a desired saccharide by disruption of polymer synthesis are: Azotobacter vinelandii/GDP-Man; Pseudomonas sp./UDP-Glc and GDP-Man; Rhizobium sp./UDP-Glc, UDP-Gal, GDP-Man; Erwinia sp./UDP-Gal, UDP-Glc; Escherichia sp./UDP-GlcNAc, UDP-Gal, CMP-NeuAc, GDP-Fuc; Klebsiella sp./UDP-Gal, UDP-GlcNAc, UDP-Glc, UDP-GlcNAc (see, e.g., Hamadeh et al. (1996) Infect. Immun. 64: 528-534); Hansenula jadiniil GDP-Man, GDP-Fuc; Candida famata/UDP-Glc, UDP-Gal, UDP-GlcNAc (Ko et al. (1996) Appl. Biochem. Biotechnol. 60: 41-48); Acetobacter xylinum/GDP-Man (Petroni et al. (1996) J. Bacteriol. 178: 4814-4121) and Saccharomyces cerevisiae/UDP-Glc, UDP-Gal, GDP-Man, GDP-GlcNAc.

Methods of introducing mutations into a target gene are well known to those of skill in the art, and are described in, for example, Ausubel, Sambrook, and Berger, all supra.

In some embodiments, the recombinant cells of the invention can produce multiple nucleotide sugars or nucleotides in addition to the sialic acid, thus allowing the introduction of multiple glycosyltransferases or multiple glycosyltransferases with supporting cycle enzymes, respectively, to produce the target sugar. This allows the production of multiple glycosidic linkages in a product using a single organism. For example, if the organism produces both UDP-Gal and UDP-GlcNAc, then addition of a Gal transferase and a GlcNAc transferase would allow the production of two new glycosidic linkages from the same organism. As another example, if an organism produces elevated levels of UTP, then by adding genes that encode enzymes for the production of UDP-Gal and UDP-GlcNAc, as well as genes that encode a Gal-transferase and a GlcNAc transferase two new glycosidic linkages can be formed from a single organism. In these examples, if the transferases allow glycosidic polymerization, then long chain oligosaccharides and polysaccharides can be formed.

### VI. Methods for Producing Product Saccharides

The invention also provides methods in which the microorganisms, including recombinant cells, of the invention are used to prepare product oligosaccharides, (which are composed of two or more saccharide residues) including sialylated product saccharides. The microorganisms used in the reaction mixtures express at least one glycosyltransferase, and, in some embodiments, accessory enzyme(s) to convert glucose taken up from the medium into a donor substrate or acceptor substrate. The culture medium then includes glucose and possibly other precursors to donor substrates or acceptor substrates. In another embodiment, the microorganisms expresses at least one sialyltransferase and a CMP-sialic acid synthase, and an enzymatic system for synthesizing sialic acid. The culture media includes an acceptor saccharide and a precursor of sialic acid, e.g., GlcNAc or pyruvate.

Those of skill will recognize that culture medium for microorganisms can be e.g., rich mediums, such as Luria broth, animal free Luria broth, or Terrific broth or synthetic medium or semi-synthetic medium, such as M9 medium.

When a sialylated product saccharide is being synthesized, one component of the growth medium is e.g., GlcNAc. Concentrations of GlcNAc can be between 0.1-200 mM. In some embodiments, GlcNAc concentrations are between 1 and 100 mM; in other embodiments, GlcNAc concentrations are between 2 and 50 mM. In a further embodiment, GlcNAc concentrations are between 5 and 15 mM. In a preferred embodiment, the GlcNAc concentration is about 10 mM.

In some embodiments, another component of the growth medium is an acceptor saccharide, e.g., lactose or glucose. Concentrations of the acceptor saccharide can be between 0.1-200 mM. In some embodiments, acceptor saccharide concentrations are between 1 and 100 mM; in other embodiments, acceptor saccharide concentrations are between 2 and 50 mM. In a further embodiment, acceptor saccharide concentrations are between 5 and 15 mM. In a preferred embodiment, the acceptor saccharide concentration is 10about mM. In some embodiments the lactose or glucose comprises a reactive moiety. In a preferred embodiment the growth medium comprises glucose-1-F. In another preferred embodiment, the growth medium comprises lactose-1-F

When both acceptor substrate and donor substrate are medium components, those of skill will recognize that the ratio of donor substrate to acceptor substrate in the medium can sometimes be optimized for oligosaccharide production, depending on the donor and acceptor substrates used and the desired oligosaccharide product. For example, when lactose and GlcNAc are included in the culture medium, and 3′sialylactose is the sialylated product, the molar ratio of GlcNAc:Lactose can range between 10:1 and 1:10. In one preferred embodiment, the molar ratio of GlcNAc:Lactose is 1:1. In a further preferred embodiment, the concentration of GlcNAc:Lactose is about 10 mM:10 mM.

In some embodiments, for production of a sialylated product saccharide, the culture medium includes pyruvate. Concentrations of pyruvate can be between 0.01-100 mM. In some embodiments, pyruvate concentrations are between 0.1 and 10 mM; in other embodiments, pyruvate concentrations are between 0.5 and 2 mM. In a preferred embodiment, the pyruvate concentration is about 1 mM.

The microorganisms, e.g., recombinant cells, of the invention are grown in culture to obtain a sufficient number of cells to produce the oligosaccharide product on a desired scale. In one embodiment, the oligosaccharide products are produced on a commercial scale. The methods of the invention are capable of producing large amounts of a desired product saccharide. For example, in some embodiments, at least 90 mg product are produced per liter of culture. In other embodiment, at least 1.6 g of product are produced intracellularly per liter and at least 1.2 grams of product are produced extracellularly per liter of culture (i.e., at least 2.8 total grams of product per liter). In other embodiments, at least 3, 4, 5, 6, 7, 10, 11, 12, 13, 14, 15, 16, 17, 18, 19, 10, or 25 grams of total product are produced per liter of culture. In some embodiments at least 20%, 30%, 40%, 50%, 60%, 70%, 75%, 80%, 85%, 90%, or 95% of the acceptor saccharide is converted to a sialylated product.

Methods and culture media for growth of microorganisms are well known to those of skill in the art. Culture can be conducted in, for example, aerated spinner or shaking culture, or, more preferably, in a fermentor.

In some embodiments, the glycosyltransferase nucleic acid and the accessory enzyme nucleic acid are under the control of an inducible promoter(s). Expression of the polypeptide(s) is then generally induced by appropriate methods, e.g., addition of inducers, such as IPTG, or changes in temperature, before harvesting the sialylated product saccharide from the cells and/or the medium. IPTG concentrations can range from between 0.01 mM to 10 mM and are preferably 0.5 mM. Other inducible promoters include e.g., the phoA promoter, which is induced on phosphate starvation, and the arabinose promoter. Those of skill will recognize that constitutive promoters can also be used in the invention.

In one embodiment, a sialyltransferase, a CMP-sialic acid synthase, and an enzymatic system for synthesizing sialic acid are under the control of an inducible promoter(s).

Upon growth of the recombinant cells to a desired cell density or to a desired level of sialylated product saccharide, the cells and medium are harvested. In some embodiments, a heterologous nucleic acids are expressed under the control of an inducible promoter. The time of induction will vary, depending on the promoter and the cells used, but will range from two hours to 240 hours. In some embodiments the induction will take place over night, e.g., between 5-20 hours. In some embodiments the induction will be performed at a different temperature then cell growth, e.g. in E. coli, cell growth can be at about 37° C. and induction can occur at a lower temperature, for example, room temperature, e.g., between 15° C. and 30° C. Cells can be subjected to concentration, and then lysed to release the sialylated product saccharide, e.g., by drying, lyophilization, treatment with surfactants, ultrasonic treatment, mechanical disruption, e.g., French press or microfluidizer, enzymatic treatment, and the like. In some embodiments, the sialylated product saccharide is isolated from the culture medium.

The products produced by the above processes can be used without purification. However, it is usually preferred to recover the product. Standard, well known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins. Nanofiltration or reverse osmosis can then be used to remove salts and/or purify the product saccharides (see, e.g., U.S. patent application Ser. No. 08/947,775, filed Oct. 9, 1997). Nanofilter membranes are a class of reverse osmosis membranes which pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

### VII. Product Oligosaccharides and Their Uses

The microorganisms, e.g., recombinant cells, and methods of the invention are useful for synthesizing a wide range of oligosaccharides that have many uses. Products that can be produced using this method include, for example, disaccharides, oligosaccharides, polysaccharides, lipopolysaccharides, glycoproteins, glycopeptides, and glycolipids including gangliosides. Any sialic acid linkage can be made using this approach, and can be combined with any glycosidic linkage. Such linkages include, but are not limited to, the addition of such sugars as fucose, sialic acid, galactose, GlcNAc, GalNAc, mannose, glucose, uronic acid forms of these sugars (e.g., glucuronic acid, galacturonic acid, etc.), xylose and fructose.

Product oligosaccharides that can be produced using the methods and reaction mixtures of the invention and are of particular interest include, but are not limited to:

A. Oligosaccharides Synthesized from Glucose in the Medium

The reaction mixtures and methods are useful for producing a wide range of oligosaccharides, a non-limiting list of oligosaccharide products is provided in Table 5.

TABLE 5
Oligosaccharide Formulas and Enzyme Activities Needed

| | Enzymes that can |
|---|---|
| Structure | be used for synthesis |
| Galβ1-4Glc | A |
| (Galβ1-4GlcNAc)ₙ where n = 1-3 | A, E |
| Fucα1-2Galβ1-4Glc | A, G |
| Galβ1-4(Fucα1-3)Glc | A, H |
| Fucα1-2Galβ1-4(Fucα1-3)Glc | A, G, H |
| Galβ1-4(Fucα1-3)GlcNAc | A, H |
| Galβ1-3(Fucα1-4)GlcNAc | B, H |
| GlcNAcβ1-3Galβ1-4Glc | A, E |
| Galβ1-4 GlcNAcβ1-3Galβ1-4Glc | A, E |
| Galα1-4Galβ1-4 GlcNAcβ1-3Galβ1-4Glc | A, C, E |
| Galβ1-3 GlcNAcβ1-3Galβ1-4Glc | A, B, E |
| Fucα1-2Galβ1-3 GlcNAcβ1-3Galβ1-4Glc | A, B, E, G |
| Galβ1-3 (Fucα1-4)GlcNAcβ1-3Galβ1-4Glc | A, B, E, H |
| Galβ1-4 (Fucα1-3)GlcNAcβ1-3Galβ1-4Glc | A, E, H |
| Galβ1-3 GlcNAcβ1-3Galβ1-4(Fucα1-3)Glc | A, B, E, H |
| Fucα1-2Galβ1-3 (Fucα1-4)GlcNAcβ1- | A, B, E, G, H |
| 3Galβ1-4Glc | |
| Galβ1-3 (Fucα1-4)GlcNAcβ1-3Galβ1- | A, B, E, H |
| 4(Fucα1-3)Glc | |
| Galα1-3Galβ1-4Glc | A, D |
| Galα1-3Galβ1-4GlcNAc | A, D |
| Galα1-3Galβ1-4 GlcNAcβ1-3Galβ1-4Glc | A, D, E |
| Siaα2-3Galβ1-4Glc | A, I |
| Siaα2-6Galβ1-4Glc | A, J |
| Siaα2-3Galβ1-4GlcNAc | A, I |
| Siaα2-6Galβ1-4GlcNAc | A, J |
| Siaα2-3Galβ1-4(Fucα1-3)Glc | A, H, I |
| Siaα2-3Galβ1-3 GlcNAcβ1-3Galβ1-4Glc | A, E, I |
| Galβ1-3 (Siaα2-6)GlcNAcβ1-3Galβ1-4Glc | A, B, E, J |
| Siaα2-6Galβ1-4 GlcNAcβ1-3Galβ1-4Glc | A, B, E, J |
| Siaα2-3Galβ1-4 GlcNAcβ1-3Galβ1-4Glc | A, B, E, I |
| Siaα2-3(Siaα2-6)Galβ1-4 GlcNAcβ1- | A, B, E, I, J |
| 3Galβ1-4Glc | |
| Siaα2-3Galβ1-4(Fucα1-3)GlcNAc | A, H, I |
| Siaα2-3Galβ1-3(Fucα1-4)GlcNAc | B, H, I |
| Galα1-4Galβ1-4Glc | A, C |
| GalNAcβ1-4Galα1-4Galβ1-4Glc | A, C, F |
| Galβ1-3GalNAcβ1-4Galα1-4Galβ1-4Glc | A, B, C, F |
| Fucα1-2Galβ1-3GalNAcβ1-4Galα1-4Galβ1- | A, B, C, F, G |
| 4Glc | |
| Siaα2-3Galβ1-3GalNAcβ1-4Galα1-4Galβ1- | A, B, C, F, G, I |
| 4Glc | |
| GalNAcβ1-3Galα1-3Galβ1-4Glc | A, D, F |
| Galβ1-3GalNAcβ1-3Galα1-3Galβ1-4Glc | A, B, D, F |
| Siaα2-3Galβ1-3GalNAcβ1-3Galα1-3Galβ1- | A, B, D, F, I |
| 4Glc | |
| Galα1-4Gal | C |
| GalNAcβ1-4Galβ1-4Glc | A, F |
| Galβ1-3GalNAcβ1-4Galβ1-4Glc | A, B, F |
| Siaα2-3Galβ1-3GalNAcβ1-4Galβ1-4Glc | A, B, F, I |
| Siaα2-3Galβ1-3(Siaα2-6)GalNAcβ1- | A, B, F, I, J |
| 4Galβ1-4Glc | |
| Siaα2-3Galβ1-3(Siaα2-8Siaα2- | A, B, F, I, J, K |
| 6)GalNAcβ1-4Galβ1-4Glc | |
| Siaα2-8Siaα2-3Galβ1-3(Siaα2-8Siaα2- | A, B, F, I, J, K |
| 6)GalNAcβ1-4Galβ1-4Glc | |
| GalNAcβ1-4(Siaα2-3)Galβ1-4Glc | A, F, I |
| Galβ1-3GalNAcβ1-4(Siaα2-3)Galβ1-4Glc | A, B, F, I |
| Siaα2-3Galβ1-3GalNAcβ1-4(Siaα2- | A, B, F, I |
| 3)Galβ1-4Glc | |
| Siaα2-8Siaα2-3Galβ1-3GalNAcβ1-4(Siaα2- | A, B, F, I, K |
| 3)Galβ1-4Glc | |
| Siaα2-8Siaα2-3Galβ1-4Glc | A, I, K |
| GalNAcβ1-4(Siaα2-8Siaα2-3)Galβ1-4Glc | A, F, I, K |
| Galβ1-3GalNAcβ1-4(Siaα2-8Siaα2- | A, B, F, I, K |
| 3)Galβ1-4Glc | |
| Siaα2-3Galβ1-3 GalNAcβ1-4(Siaα2-8Siaα2- | A, B, F, I, K |
| 3)Gal β1-4Glc | |
| Siaα2-8Siaα2-3Galβ1-3GalNAcβ1-4(Siaα2- | A, B, F, I, K |
| 8Siaα2-3)Galβ1-4Glc | |
| Siaα2-8Siaα2-8Siaα2-3Galβ1-4Glc | A, I, K |
| GalNAcβ1-4(Siaα2-8Siaα2-8Siaα2- | A, F, I, K |
| 3)Galβ1-4Glc | |
| Galβ1-3GalNAcβ1-4(Siaα2-8Siaα2-8Siaα2- | A, B, F, I, K |
| 3)Galβ1-4Glc | |
| Siaα2-3Galβ1-3GalNAcβ1-4(Siaα2-8Siaα2- | A, B, F, I, K |
| 8Siaα2-3)Galβ1-4Glc | |
| Fucα1-2Galβ1-3GalNAcβ1-4(Siaα2- | A, B, F, G, I |
| 3)Galβ1-4Glc | |

Key:
A = β1,4Galactosyltransferase (e.g., lgtB-Neisseria meningitidis/gonorrhoeae)
B = β1,3Galactsoyltransferase (e.g., cgtB-Campylobacter jejuni)
C = α1,4Galactosyltraferase (e.g., lgtC-Neisseria meningitidis/gonorrhoeae)
D = α1,3Galactosaminyltransferase (e.g., mouse or bovine enzyme)
E = β1,3N-actylglucosaminyltransferase (e.g., lgtA-Neisseria meningitidis/gonorrhoeae)
F = β1,4N-acetylgalactosaminyltransferase (e.g., cgtA-Campylobacter jejuni)
G = α1,2Fucosyltransferase (e.g., futC-H. pylori)
H = α1,3/4Fucosyltransferase (e.g., futA/b-H. pylori)
I = α2,3Sialyltransferase (e.g., lst Neisseria meningitidis/gonorrhoeae; CstI, CstII, or CstIII Campylobacter)
J = α2,6Sialyltransferase (e.g., Photobacterium)
K = α2,8Sialyltransferase (e.g., CstII, Campylobacter)

Generally, the user will decide on an oligosaccharide product for synthesis and then select appropriate acceptor substrates and donor substrates for use in the methods of the invention. The choice of oligosaccharide product and substrates will allow the user to select accessory enzyme(s) and glycosyltransferase(s) for use in the invention.

In some embodiments, the oligosaccharides or a portion of the oligosaccharides are synthesized from glucose in the growth medium. After being taken up by the host cells, glucose can be used by the cells as an acceptor saccharide for synthesis of an oligosaccharide or a precursor of an oligosaccharide, or as a donor saccharide after activation by an appropriate enzyme to form UDP-Glu, or as a precursor of a different donor saccharide, e.g., UDP-Gal, UDP-GalNAc, or UDP-GlcNAc.

In some embodiments, glucose is used an acceptor saccharide. Many examples of oligosaccharides that comprise glucose as an acceptor saccharide are found in Table 4.

For example, a β1,4-galactosyltransferase gene (e.g., lgtB gene from Neisseria, Campylobacter or Haemophilus) and UDP-glucose-4′epimerase gene (e.g., Streptococcus thermophilus (Accession number M38175), rat, Pseudomonas) are expressed in a host cell. The β1,4-galactosyltransferase gene and UDP-glucose-4′epimerase gene can be heterologous genes and are then expressed on the same plasmid, on different plasmids, or are integrated into the bacterial cell genome. The β1,4-galactosyltransferase gene and UDP-glucose-4′epimerase gene can be expressed under the control of the same or different promoters. The β1,4-galactosyltransferase gene and UDP-glucose-4′epimerase gene can be expressed as two separate proteins or can be joined to form a fusion protein. See, e.g., PCT/CA98/01180 and Chen et al., J. Biol. Chem. 275:31594-31600 (2000)). The cells produce UDP-galactose from UDP-glucose using the activity of the UDP-glucose-4′ epimerase protein. If the cells are unable to produce sufficient galactose, galactose can be added to the growth medium to facilitate synthesis of UDP-galactose. The β1,4-galactosyltransferase catalyzes the in vivo formation of lactose from glucose and UDP-galactose.

Once lactose has been made, oligosaccharides that comprise lactose can be synthesized by addition of appropriate additional glycosyltransferase(s) and/or accessory enzyme(s) can be added to the cells, depending on the selected oligosaccharide. For example, sialylated oligosaccharides can be made using the disclosed methods. A host cell is constructed to allow production of sialyl-lactose (SL) from glucose and other precursors in growth medium. The bacterial strain comprises a β1,4-galactosyltransferase gene and a UDP-glucose-4′epimerase gene, as described above. At a minimum, the host cell also comprises a nucleic acid that encodes a sialyltransferase. The host cell can also comprise a gene that encodes a CMP-NAN synthetase (e.g., neuA, siaB) polypeptide. If the host cell is unable to produce sufficient sialic acid endogenously, an enzymatic system for synthesis of sialic acid can also be expressed in the host cell, e.g., an N-acetylglucosamine 6-phosphate-2′-epimerase (e.g., siaA, neuC), and a gene encoding an activity to allow condensation of N-acetylmannosamine and phophoenolpyruvate and generate sialic acid (e.g., neuB, siaC genes). Where an enzymatic system to produce sialic acid is inserted into the cell, it can be beneficial to add precursors of sialic acid to the medium, e.g., the following genes: CMP-NAN synthetase (e.g., neuA, siaB), an α2,3-sialyltransferase (e.g., from N. meningitides, N. gonorrhoea, Campylobacter, Haemophilis, or Pasteurella), an N-acetylglucosamine 6-phosphate-2′-epimerase (e.g., siaA, neuC), and a gene encoding an activity to allow condensation of N-acetylmannosamine and phophoenolpyruvate and generate sialic acid (e.g., neuB, siaC genes). The CMP-NAN synthetase and the α2,3-sialyltransferase can be expressed as two separate proteins or as a fusion protein. For synthesis of sialic acid, a sialic acid operon or gene cluster from a bacterial strain can be transformed into the host cell, e.g., the entire region 2 of the kps gene cluster from E. coli K1 (NeuD, NeuB, NeuA, NeuC, NeuF, and NeuS) or the polysialic acid capsule gene cluster from Neisseria or Campylobacter. The sialic acid operon or gene cluster can be cloned into an expression vector or stably incorporated into the genome of the host cell.

Additional sugar residues can be added to make a product oligosaccharide. For example, beginning with the sialylactose synthesis described above, a GM1 oligosaccharide (Gal3GalNAc4(Neu5Ac3)Gal4Glc) can be synthesized by adding genes that encode a β1,4N-acetylgalactosaminyltransferase and a β1,3galactosaminyltransferase into the host bacteria. The β1,4N-acetylgalactosaminyltransferase and the β1,3galactosaminyltransferase are known to those of skill, e.g., Campylobacter genes, i.e., encoded by cgtA and cgtB genes. Other sugar residues can be included using, e.g., the enzymes listed in Table 4.

Because of the central position of glucose in sugar metabolic pathways, oligosaccharides that do not comprise glucose can by synthesized from glucose in the growth medium. In some embodiments glucose is converted to another sugar, e.g., GlcNAc or Gal, which is then used as an acceptor saccharide.

Any suitable host cell can be used, e.g., E. coli, or Bacillus subtilis. The bacteria are typically cultured in a defined growth medium, e.g., M9 supplemented with glucose, and glucose is taken up by the cell (e.g., active or passive transport, endocytosis, transporter mediated).

In additional embodiments enzymes that allow addition of fucose to a product oligosaccharide are found in the host cells. The host cells can include a fucosyltransferase as described herein. If the host cells are unable to produce sufficient fucose, appropriate accessory enzymes or an enzymatic system for fucose synthesis can be inserted into the cells. In one embodiment, the host cells are genetically manipulated to enhance fucose production. For example, fucose is an intermediate in colonic acid production by E. coli. Fucose synthesis is enhance by manipulation of the genes encoding the colonic acid biosynthetic pathway. A gene the encodes a positive regulator of the colonic acid operon (e.g., rcsA) can be overexpressed, while the downstream wcaJ gene can be inactivated to divert the fucose intermediate from the production of colonic acid. See, e.g., Dumon et al., Glyconjugate J. 18:465-474 (2001). A fucose residue is added to the product oligosaccharide by an appropriate fucosyltransferase.

In preferred embodiments, a fucosylated, sialylated oligosaccharide product is synthesized in host cells that comprise a fucosyltransferase, a sialyltransferase, an enzymatic system for synthesizing fucose, and an enzymatic system for synthesizing sialic acid.

B. Synthesis of Sialylated Product Saccharides

In addition to the methods described above, where sialylated product saccharides are synthesized using glucose or a monosaccharide derived from glucose as a first acceptor substrate, sialylated product saccharides can also be synthesized using lactose or other di- or tri- or oligosaccharides as acceptor saccharides. The acceptor saccharides are components of the growth medium and are taken up by the cells, acted on by glycosyltransferases in the cells, including sialyltransferases, to form the desired sialylated product saccharide.

In a preferred embodiment, lactose is a component of the growth medium and is taken up by the cells. In some embodiments, the cells are genetically manipulated to enhance use of lactose. For example, where lactose is the acceptor saccharide, E. coli cells that express or overexpress the LacY permease can be used. Also in E. coli, when lactose is the acceptor saccharide or an intermediate in synthesizing the sialylated product, lactose breakdown can be minimized by using host cells that are LacZ-.

In preferred embodiments, the host cells comprise a nucleic acid that encodes CMP-NAN synthetase and a nucleic acid that encodes a sialyltransferase. The CMP-NAN synthetase and the sialyltransferase can be expressed as a fusion protein. The host cell also preferably include an enzymatic system for synthesis of sialic acid, e.g., from inexpensive precursors in the growth medium such as N-acetylglucosamine and/or pyruvate. Examples of enzymatic systems for synthesis of sialic acid are e.g., from Neisseria, a GlcNAc epimerase (the SiaA protein, Accession Number M95053 region: 174.1307) and an N-acetyl neuraminic acid (NANA) condensing polypeptide (the SiaC protein, Accession Number M95053 region: 1998.3047), and from E. coli K12, UDP-GlcNAc epimerase (the NeuC protein, Accession number M84026), NeuB gene product (a sialate synthase protein, Accession number AAC43302, encoded by Accession number U05248, region 723-1763), and the NeuA gene product (a CMP-sialate synthase protein, Accession number J05023). See, e.g., Ringenberg et al., Glycobiology 11:533-539 (2001). In some embodiments, the host cell further comprises a heterologous CTP synthetase polypeptide.

In some embodiments 3′sialylactose is synthesized using the methods disclosed herein. Other sialylated product saccharides can be synthesized, including those found in Table 5. The additional glycosyltransferases that may be required are also listed in Table 5 Accessory enzymes can also be included to enhance production of a donor substrate of a glycosyltransferase that is not a sialyltransferase. One preferred group of sialylated product saccharides are headgroups of glycolipids, gangliosides and related structures shown in Table 6.

In another preferred embodiment a fucose residue is added to a sialylated product saccharide according to the methods described herein. Thus, the host cells can be grown on a growth medium that includes an acceptor sugar, e.g., lactose, and precursors of sialic acid and/or fucose. The host cells comprise enzymatic systems for synthesizing sialic acid or fucose and in some embodiments for synthesizing activated forms of those sugars that serve as donor substrates. The host cells also comprise a sialyltransferase and a fucosyltransferase and one or both of these enzymes are heterologous enzymes.

TABLE 6
Ganglioside Formulas and Abbreviations

| | Abbre- |
|---|---|
| Structure | viation |
| Neu5Ac3Gal4GlcCer | GM3 |
| GalNAc4(Neu5Ac3)Gal4GlcCer | GM2 |
| Gal3GalNAc4(Neu5Ac3)Gal4GlcCer | GM1a |
| Neu5Ac3Gal3GalNAc4Gal4GlcCer | GM1b |
| Neu5Ac8Neu5Ac3Gal4GlcCer | GD3 |
| GalNAc4(Neu5Ac8Neu5Ac3)Gal4GlcCer | GD2 |
| Neu5Ac3Gal3GalNAc4(Neu5Ac3)Gal4GlcCer | GD1a |
| Neu5Ac3Gal3(Neu5Ac6)GalNAc4Gal4GlcCer | GD1α |
| Gal3GalNAc4(Neu5Ac8Neu5Ac3)Gal4GlcCer | GD1b |
| Neu5Ac8Neu5Ac3Gal3GalNAc4(Neu5Ac3)Gal4GlcCer | GT1a |
| Neu5Ac3Gal3GalNAc4(Neu5Ac8Neu5Ac3)Gal4GlcCer | GT1b |
| Gal3GalNAc4(Neu5Ac8Neu5Ac8Neu5Ac3)Gal4GlcCer | GT1c |
| Neu5Ac8Neu5Ac3Gal3GalNAc4(Neu5Ac8Neu5c3)Gal4GlcCer | GQ1b |

Nomenclature of Glycolipids, IUPAC-IUB Joint Commission on Biochemical Nomenclature (Recommendations 1997); Pure Appl. Chem. (1997) 69: 2475-2487; Eur. J. Biochem (1998) 257: 293-298) (www.chem.qmw.ac.uk/iupac/misc/glylp.html).

C. Pharmaceutical and Other Applications

The compounds described above can then be used in a variety of applications, e.g., as antigens, diagnostic reagents, foodstuffs, or as therapeutics. Thus, the present invention also provides pharmaceutical compositions which can be used in treating a variety of conditions. The pharmaceutical compositions are comprised of oligosaccharides made according to the methods described above.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the oligosaccharides of the invention can be incorporated into liposomes formed from standard vesicle-forming lipids. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

The compositions containing the oligosaccharides can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 40 g of oligosaccharide per day for a 70 kg patient, with dosages of from about 5 mg to about 20 g of the compounds per day being more commonly used.

Single or multiple administrations of the compositions can be carried out with dose levels and pattern being selected by the treating physician. In any event, the pharmaceutical formulations should provide a quantity of the oligosaccharides of this invention sufficient to effectively treat the patient.

The oligosaccharides may also find use as diagnostic reagents. For example, labeled compounds can be used to locate areas of inflammation or tumor metastasis in a patient suspected of having an inflammation. For this use, the compounds can be labeled with appropriate radioisotopes, for example, ¹²⁵I, ¹⁴C, or tritium.

The oligosaccharide of the invention can be used as an immunogen for the production of monoclonal or polyclonal antibodies specifically reactive with the compounds of the invention. The multitude of techniques available to those skilled in the art for production and manipulation of various immunoglobulin molecules can be used in the present invention. Antibodies may be produced by a variety of means well known to those of skill in the art.

The production of non-human monoclonal antibodies, e.g., murine, lagomorpha, equine, etc., is well known and may be accomplished by, for example, immunizing the animal with a preparation containing the oligosaccharide of the invention. Antibody-producing cells obtained from the immunized animals are immortalized and screened, or screened first for the production of the desired antibody and then immortalized. For a discussion of general procedures of monoclonal antibody production, see, Harlow and Lane, Antibodies, A Laboratory Manual, Cold Spring Harbor Publications, N.Y. (1988).

It must be noted that as used herein and in the appended claims, the singular forms “a”, “and”, and “the” include plural referents unless the context clearly dictates otherwise. Thus, for example, reference to “a bacteriophage” includes a plurality of such bacteriophage and reference to “the host bacterium” includes reference to one or more host bacteria and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations are incorporated herein by reference.

### EXAMPLES

### Example 1

### Generation of Plasmids and Host Strains for Synthesis of Sialylated Products

Host strains for production of sialylated products were constructed by transforming an E. coli strain, JM109, with a plasmid encoding four enzymes involved in sialylation. The four enzymes were SiaA (GlcNAc epimerase from Neisseria), SiaC (NAN condensing enzyme from Neisseria), ST (Sialyltransferase from Neisseria) and CNS (CMP-NAN synthetase from Neisseria). The ST and CNS were expressed as a fusion protein. (See, e.g., WP99/31224 and Gilbert et al., Nat. Biotechnol. 16:769-72 (1998)).

Two plasmids were constructed. The first used the pNT1-RMK plasmid as a starting plasmid; the second used pcWIN2 as a starting plasmid. Both plasmids have expression cassettes with lacZ promoters that are induced on addition of compounds such as IPTG. The pNT1-RMK plasmid was constructed first. Six PCR primers were designed to add 5′ and 3′ restriction sites and 5′ ribosomal binding sites (RBS) to the SiaC nucleic acid and the ST/CNS fusion nucleic acid. SiaA did not include a RBS because it forms a fusion with RMK (rabbit myokinase) from the pNT1-RMK vector. SiaA was designed with a 5′ BamHI site and a 3′ SacI site. SiaC was designed with a 5′ HindIII site and a 3′ XbaI site. CNS/ST was designed with a 5′ XbaI site and a 3′ MluI site.

Three PCR reactions were setup each with 1 μL 10 mM dNTP Mix, 1 μL NEB 10× ThermoPol Buffer, 81 μL Gibco/BRL DNAse Free H₂O and 1 μL of the appropriate DNA template. The reactions were run in the Thermocycler under the following conditions:

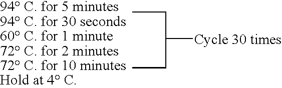

After the initial 5 minutes at 94° C., the program was paused and 1 μL VENT Polymerase was added to each reaction. The program was then continued and allowed to run to completion. PCR products were isolated and ligated together with the pNT1-RMK plasmid, to make the pNT1-RMK SiaA SiaC CNS/ST plasmid. The inserts from the pNT1-RMK SiaA SiaC CNS/ST plasmid were then used to construct a similar plasmid using a pCWIN2 backbone, the pCWIN2 SiaA SiaC CNS/ST plasmid. Both plasmids were used to transform E. coli strain JM109, which is a lacZ- strain. Transformants were selected using kanamycin.

### Example 2

### Synthesis of 3′-Sialylactose

A JM109 pNT1-RMK SiaA SiaC CNS/ST colony was inoculated into 2 mL animal free LB culture, containing 201g/mL kanamycin sulfate, and incubated overnight at 37° C., 250 rpm. A 400 mL animal free LB culture, containing 201g/mL kanamycin sulfate, was inoculated with 400 μL of the JM109 pNT1-RMK SiaA SiaC CNS/ST starter culture. This culture was grown approximately 5 hours and the OD600 was measured by UV Spectrophotometer and found to be mid-log (e.g., 0.2-1.5 OD). Four milliliters of 100 mM GlcNAc (final concentration 1 mM) and 8 mL 500 mM Lactose (final concentration 10 mM) was added to the culture, as well as 400 μL IPTG (final concentration 0.5 mM) to induce the culture. The culture was removed from the 37° C. incubator and placed in the 25° C. incubator at 250 rpm, overnight.

The 400 mL JM109 pNT1-RMK SiaA SiaC CNS/ST culture was removed from the incubator and divided into two 250 mL centrifuge bottles. The culture was centrifuged at 6000 rpm, at 4° C. for 15 minutes. The supernatant was removed and four 1 mL aliquots were saved for analysis. The pellet was resuspended in 3.3 mL water per gram, for a total volume of 25 mL resuspended pellet, and lysed using a French Press at 1200 psi.

A 1 mL aliquot of JM109 pNT1-RMK SiaA SiaC CNS/ST lysate and JM109 pNT1-RMK SiaA SiaC CNS/ST culture supernatant were taken. The JM109 pNT1-RMK SiaA SiaC CNS/ST lysate sample was centrifuged at 14000 rpm for 10 minutes. One μL of the cleared lysate and of supernatant were spotted on a TLC plate along with 1 μL of the following standards: 10 mM Lactose, 10 mM NAN, 50 mM IPTG, 5 mM 3′SL, 50 mM GlcNAc, 50 mM ManNAc and 50mM GlcNAc-6-P. The plate was run in 70:20:10 IPA:H2O:Acetic Acid for approximately 1 hour. The plate was then dried, dipped in anisaldehyde and heated to develop stain. Samples were also analyzed by TLC using resorcinol stain. The plate was run using the same solvent, but resorcinol stain was sprayed on the plate and it was heated with a glass cover to develop. Samples of JM109 pCWIN2 SiaA SiaC CNS/ST cells were also grown, harvested and analyzed as described above.

E. coli cells that expressed pNT1-RMK SiaA SiaC CNS/ST or pCWIN2 SiaA SiaC CNS/ST were able to synthesize 3′-sialylactose (3′SL), while cells transformed with a control plasmid did not. 3′SL was recovered in lysates, in cleared lysates, and in the culture supernatant from the cells expressing pNT1-RMK SiaA SiaC CNS/ST or pCWIN2 SiaA SiaC CNS/ST. Under some conditions, cells transformed with pCWIN2 SiaA SiaC CNS/ST appeared to produce more 3′SL.

### Example 3

### Optimization of 3′Sialylactose Production

### Growth Conditions

The effects of concentration of four intermediates in the production of 3′SL by JM109 E. coli transformed with pNT1-RMK SiaA SiaC CNS/ST were investigated. Concentrations of N-acetyl glucosamine (GlcNAc), pyruvate, lactose and cytosine triphosphate (CTP) were varied. Cultures were inoculated with 200 μL (for 200 mL cultures) or 1 mL (for 1 L cultures) of culture from a 20 μg/mL Kanamycin sulfate animal free LB starter culture of JM109 pNT1-RMK SiaA SiaC CNS/ST. Cultures were incubated for about 5 hours at 37° C., 250 rpm. The culture density was monitored for each culture by measuring OD₆₀₀ on UV Spectrophotometer. The cultures were all induced in a range of 0.7≦OD₆₀₀≦1.2 with the specified amounts of GlcNAc, Lactose, IPTG, Pyruvate and CTP as shown in Table 7.

TABLE 7
JM109 pNT1-RMK SiaA SiaC CNS/ST Culture Growth Experiments

| | Volume Added (mL) | | | | | | | | | |
|---|---|---|---|---|---|---|---|---|---|---|
| Experiment | GlcNAc | Lactose | IPTG | | | Component Final Concentration (mM) | | | | |
| (Volume) | (100 mM) | (500 mM) | (500 mM) | Pyruvate | CTP | GlcNAc | Lactose | IPTG | Pyruvate | CTP |
| JM109 | 2 | 20* | 0.200 | | | 1 | 50* | 0.5 | | |
| Parental | | | | | | | | | | |
| 1 (200 mL) | 2 | 20* | 0.200 | | | 1 | 50* | 0.5 | | |
| 2 (200 mL) | 4 | 4 | 0.200 | | | 2 | 10 | 0.5 | | |
| 3 (200 mL) | 20 | 4 | 0.200 | | | 10 | 10 | 0.5 | | |
| 4 (200 mL) | 4 | 4 | 0.200 | | | 10 | 10 | 0.5 | | |
| 5 (200 mL) | 8 | 4 | 0.200 | | | 20 | 10 | 0.5 | | |
| 6 (200 mL) | 40 | 4 | 0.200 | | | 100 | 10 | 0.5 | | |
| 7 (200 mL) | 0.4 | 0.4 | 0.200 | | | 1 | 1 | 0.5 | | |
| 8 (200 mL) | 2 | 2 | 0.200 | | | 5 | 5 | 0.5 | | |
| 9 (200 mL) | 4 | 4 | 0.200 | | | 10 | 10 | 0.5 | | |
| 10 (200 mL) | 4 | 4 | 0.200 | | 0.4 | 10 | 10 | 0.5 | | 1 |
| 11 (200 mL) | 4 | 4 | 0.200 | 0.4 | | 10 | 10 | 0.5 | 1 | |
| 12 (200 mL) | 4 | 4 | 0.200 | 0.4 | 0.4 | 10 | 10 | 0.5 | 1 | 1 |
| 13 (1 L) | 20 | 20 | 1 | 2 | | 10 | 10 | 0.5 | 1 | |
| 14 (1 L)** | 20 | 20 | 1 | 2 | | 10 | 10 | 0.5 | 1 | |
| 15 (2 × 1 L) | 20 | 20 | 1 | 2 | | 10 | 10 | 0.5 | 1 | |

*In these two cases 20 mL of 500 mM Lactose was added for a final concentration of 50 mM.
**This culture was prepared using M9CA M9 Salts media from Teknova for the culture media in place of the animal free LB media.

Cultures were harvested by centrifuging at 6000 rpm for 30 minutes at 4° C. The pellets were resuspended in 3.3 mL of water per gram, and were then lysed by French Press. One mL aliquots were taken from the lysates and centrifuged at 14,000 rpm for 10 minutes to clear the lysate for analysis.

Two μL of the cleared lysate were spotted on a TLC plate along with 1 μL of standards as above. The plates were run in 70:20:10 IPA:H2O:Acetic Acid for approximately 1 hour. The plates were then dried, sprayed with Resorcinol and heated to develop stain.

Based on TLC analysis, 3′SL was produced under all experimental conditions tested. Based on the intensity of 3′SL bands in small scale experiments, JM109 E. coli transformed with pNT1-RMK SiaA SiaC CNS/ST appeared to produce the highest levels of 3′SL when grown on animal free LB with 10 mM Lactose, 10 mM GlcNAc, 0.5 mM IPTG and 1 mM Pyruvate. A 1:1 ratio of lactose to GlcNAc gave good production of 3′SL and a 10 mM:10 mM ratio of lactose to GlcNAc provided the best production of 3′SL, using this strain of E. coli. Addition of pyruvate to the culture also seemed to increase production of 3′SL. Finally, CTP apparently had an inhibitory effect from the decrease in intensity of 3′SL in those cultures.

Based on these results, the larger scale experiments 13 through 15, e.g., 1 L cultures, were grown with these conditions. Scaling the cultures up from 200 mL to 1000 mL provided evidence that this fermentation process is scaleable. The levels of 3′SL in the culture remained constant as the culture volume was increased.

### Purification of 3′SL

3′SL from a JM109 pNT1-RMK SiaA SiaC CNS/ST 1 liter culture was purified using charcoal. Lysate from JM109 pNT1-RMK SiaA SiaC CNS/ST 1 L culture was diluted to 400 mL with RO H₂O. 48 g of activated charcoal (Norit) and 48 g Celite were combined in a beaker and the 400 mL lysate sample was applied. The mixture was stirred with a stir bar on a stir plate for about 15 minutes. The sample/charcoal/celite slurry was applied to a column with vacuum applied. The slurry was washed with 2×400 mL H₂O. The sample was then eluted from charcoal/celite with 4×400 mL 50:50 v/v EtOH: H₂O. All four elutions were collected and saved as separate fractions. Fractions were concentrated by rotovap to approximately 50 mL.

The four concentrated fractions were then analyzed by TLC. Two μL of the each concentrated fraction was spotted on a TLC plate along with 1 μL of the following standards: 10 mM Lactose, 10 mM NAN, 5 mM 3′SL, 50 mM GlcNAc and 50 mM ManNAc. The plate was run in 70:20:10 IPA:H2O:Acetic Acid for approximately 1 hour. The plate was then dried, sprayed with Resorcinol and heated with a glass cover to develop stain.

All four fractions were pooled for lyophilization. The concentrated fractions were quick frozen to the cylinder by rotating the cylinder in an acetone/dry ice bath. The sample was lyophilized at <500 mT and <−40° C. for approximately 16 hours.

The lyophilized material was resuspended in 6 mL RO H₂O. The resulting mixture had some insoluble material in it that most likely was celite. This material was allowed to settle out and 300 μL of the upper layer was analyzed by capillary electrophoresis (CE) method. Results from this analysis, indicated that from the total lysate of the 1 L culture, approximately 90 mg of 3′SL were isolated. In addition this sample was analyzed by HPLC. The concentration of 3′SL was approximately 100 mg/L, which correlated well to the CE results.

3′SL was also purified by nanofiltration. Two JM109 pNT1-RMK SiaA SiaC CNS/ST 1 liter cultures were homogenized by two passages through the homogenizer at 9000 psi. Approximately 700 mL H₂O were used to recover any culture held up in the homogenizer void volume. The pH of the culture was then determined to be approximately 5 and was adjusted to ˜6.5 with 5N NaOH. The culture was then transferred equally to two 1 L water jacketed glass vessels at about 90° C. to precipitate any easily removable solids, e.g., proteins. The cultures were also stirred using a magnetic stir bar. The culture reached 85° C. in approximately 1.5 hours. Solids, most likely proteins, were observed in the cultures. The culture was then equally aliquoted to six 500 mL centrifuge bottles. Culture was centrifuged at 5000 rpm, 4° C. for 30 minutes to remove solids. The resulting supernatant was processed through a 0.22 μm vacuum filter.

The permeate was passed through a 10K Pelicon PLCGC Mini 0.1 m² filtration area Hollow Fiber Filter and the permeate was collected. The processing time for the 3 L sample was about 1 hour. Nanofiltration was then performed using a flat sheet tester. An Osmonics GE membrane was then cut to fit the tester, and was placed on the tester. The tester housing was then tightened by use of a torque wrench and set at 200 in/lbs. The system was then flushed with RO water. Permeate from 10K filtration was processed on the GE membrane running at approximately 350 psi. The total input volume was processed down to about 300 mL in approximately 7 hours.

The concentrated retentate was diafiltered four times with 250 mL of H₂O. For each diafiltration, the H₂O was added, the sample was processed back down to 250 mL total volume and the next diafiltration volume was added. At the end of the processing, the 300 mL of retentate was collected as the final sample.

The retentate was analyzed by TLC as in the experiments before. Two μL of retentate was spotted on a TLC plate along with 1 μL of the following standards: 10 mM Lactose, 10 mM NAN, 5 mM 3′SL, 50 mM GlcNAc and 50mM ManNAc. The plate was run in 70:20:10 IPA:H₂O:Acetic Acid for approximately 1 hour. The plate was then dried, sprayed with Resorcinol and heated covered to develop stain. The sample was also analyzed by HPLC. Results of pooled sample analyzed by CE indicated that from the total lysate of the 1 L culture, approximately 90 mg of 3′SL were isolated. In addition results of HPLC analysis determined that the concentration of 3′SL was approximately 100 mg/L.

### Example 4

### Production of Oligosaccharides Using Glucose as a Starting Material

Glucose is added to the medium for growth of an appropriate bacterial strain. After being taken up by the bacterial cells, glucose is used by the cells as an acceptor saccharide for synthesis of an oligosaccharide or a precursor of an oligosaccharide, or as a donor saccharide after activation by an appropriate enzyme to form GDP-Glu, or as a precursor of a different donor saccharide, e.g., UDP-Gal, UDP-GalNAc, or UDP-GlcNAc.

A. Production of Lactose in Bacteria Using Glucose as a Starting Material

An appropriate strain of bacteria is constructed to allow expression of a β1,4-galactosyltransferase gene (e.g., lgtb gene from Neisseria, Campylobacter or Haemophilus) and UDP-glucose-4′epimerase gene (e.g., rat, Pseudomonas). The β1,4-galactosyltransferase gene and UDP-glucose-4′epimerase gene can be heterologous genes and are then expressed on the same plasmid, on different plasmids, or are integrated into the bacterial cell genome. The β1,4-galactosyltransferase gene and UDP-glucose-4′epimerase gene can be expressed under the control of the same or different promoters. The β1,4-galactosyltransferase gene and UDP-glucose-4′epimerase gene can be expressed as two separate proteins or can be joined to form a fusion protein.

The bacteria are E. coli strain, e.g., K12 or other bacteria, e.g., Bacillus subtilis. The bacteria are typically cultured in a defined growth medium, e.g., M9 supplemented with glucose, and glucose is taken up by the cell (e.g., active or passive transport, endocytosis, transporter mediated). The cells produce UDP-galactose from glucose using the UDP-glucose-4′epimerase protein. In other embodiments galactose is also added to the growth medium to facilitate synthesis of UDP-galactose. The β1,4-galactosyltransferase catalyzes the in vivo formation of lactose from glucose and UDP-galactose.

The synthesis of lactose is monitored by assaying aliquots of culture medium or cells by chromatography, mass spec, or other methods to detect oligosaccharides. Lactose is purified from the culture medium or from bacterial cells after harvest.

B. Production of 3′ Sialyl-Lactose in Bacteria Using Glucose as a Starting Material

A bacterial strain is constructed to allow production of sialyl-lactose (SL) from glucose and other precursors in growth medium. The bacterial strain comprises a β1,4-galactosyltransferase gene and a UDP-glucose-4′epimerase gene as described above. In addition the bacteria comprise the following genes: CMP-NAN synthetase (e.g., neuA, siaB), an α2,3-sialyltransferase (e.g., from N. meningitides, N. gonorrhoea, Campylobacter, Haemophilis, or Pasteurella), an N-acetylglucosamine 6-phosphate-2′-epimerase (e.g., siaA, neuC), and a gene encoding an activity to allow condensation of N-acetylmannosamine and phophoenolpyruvate and generate sialic acid (e.g., neuB, siaC genes). The genes for synthesis of SL are endogenous or heterologous genes. For example, the plasmids of Example 1, above, are used. The genes for synthesis of SL are included in expression vectors or are part of an expression construct that is integrated into the host genome. The CMP-NAN synthetase and the α2,3-sialyltransferase can be expressed as two separate proteins or as a fusion protein. For synthesis of sialic acid, a sialic acid operon or gene cluster from a bacterial strain can be transformed into the host cell, e.g., the entire region 2 of the kps gene cluster from E. coli K1 (NeuD, NeuB, NeuA, NeuC, NeuF, and NeuS) or the polysialic acid capsule gene cluster from Neisseria or Campylobacter. The sialic acid operon or gene cluster can be cloned into an expression vector or stably incorporated into the genome of the host cell.

After construction of appropriate expression constructs or vectors, the constructs or vectors are transformed into a host cell or integrated into a host genome. As above, the bacteria are E. coli strain, e.g., K12 or other bacteria, e.g., Bacillus subtilis. The bacteria are typically cultured in a defined growth medium, e.g., M9 supplemented with glucose, N-acetylglucosamine and pyruvate. After synthesis of lactose as above, the lactose is then sialylated using the CMP-NAN generated from N-acetylglucosamine, pyruvate and the products of the N-acetylglucosamine 6-phosphate-2′-epimerase, the N-acetylneuraminic acid synthase and the α2,3sialyltransferase.

The synthesis of SL is monitored by assaying aliquots of culture medium or cells by chromatography, mass spec, or other methods to detect oligosaccharides. SL is purified from the culture medium or from bacterial cells after harvest.

C. Production of GM1 Oligosaccharide in Bacteria Using Glucose as a Starting Material

Bacterial strains are constructed for production of SL as described above. GM1 oligosaccharide has the following formula: Gal3GalNAc4(Neu5Ac3)Gal4Glc. Thus, expression vectors or expression constructs comprising genes that encode β1,4N-acetylgalactosaminyltransferase and the β1,3galactosaminyltransferase are also transformed or integrated into the host bacteria. The β1,4N-acetylgalactosaminyltransferase and the β1,3galactosaminyltransferase are from a Campylobacter species, i.e., encoded by cgtA and cgtB genes, respectively.

The synthesis of GM1 is monitored by assaying aliquots of culture medium or cells by chromatography, mass spec, or other methods to detect oligosaccharides. GM1 is purified from the culture medium and/or from bacterial cells after harvest.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of producing an oligosaccharide, the method comprising the step of culturing a microorganism in a culture medium comprising a glucose moiety, wherein the microorganism comprises
a heterologous galactosyltransferase activity, wherein the galactosyltransferase activity catalyzes the transfer of a galactose moiety from an activated galactose molecule to the glucose moiety to form a disaccharide.

2. The method of claim 1, wherein the microorganism further comprises a heterologous enzymatic system for synthesizing an activated galactose moiety from glucose.

3. The method of claim 1, wherein the oligosaccharide is lactose.

4. The method of claim 1, wherein the glucose moiety is glucose or N-acetylglucosamine (GlcNAc).

5. The method of claim 1, wherein the galactosyltransferase activity is selected from the group consisting of β1,3-galactosyltransferase and β1,4-galactosyltransferase.

6. The method of claim 1, wherein the microorganism further comprises a second heterologous glycosyltransferase, wherein the second glycosyltransferase catalyzes transfer of an activated sugar moiety to the disaccharide.

7. The method of claim 6, wherein the second heterologous glycosyltransferase is a sialyltransferase.

8. The method of claim 7, further comprising a heterologous fucosyltransferase.

9. The method of claim 7, wherein the culture medium further comprises N-acetylglucosamine (GlcNAc),
wherein the microorganism further comprises
b) an enzymatic system for synthesizing sialic acid from N-acetylglucosamine,
c) a CMP-sialic acid synthase polypeptide, and
d) a sialyltransferase polypeptide, wherein culture takes places under conditions suitable for synthesizing an activated sialic acid molecule and wherein the sialyltransferase polypeptide catalyzes the transfer of a sialic acid moiety from the activated sialic acid molecule to the lactose moiety to produce the oligosaccharide.

10. The method of claim 6, wherein the second heterologous glycosyltransferase is selected from the group consisting of a fucosyltransferase, an N-acetylglucosaminyl (GlcNAc) transferase, an N-acetylgalactosaminyl (GalNAc) transferase, and an α1,4-galactosyltransferase.

11. A method of producing a sialylated product saccharide, said method comprising the step of:
growing a microorganism in a culture media comprising N-acetylglucosamine and an acceptor substrate, wherein said microorganism comprises:
a) an enzymatic system for synthesizing sialic acid from N-acetylglucosamine,
b) a CMP-sialic acid synthase polypeptide, and
c) a sialyltransferase polypeptide, wherein growth takes places under conditions suitable for synthesizing an activated sialic acid molecule and wherein the sialyltransferase polypeptide catalyzes the transfer of a sialic acid moiety from the activated sialic acid molecule to the acceptor substrate to produce the sialylated product saccharide.

12. The method of claim 11, wherein the enzymatic system for synthesizing sialic acid comprises an N-acetylglucosamine (GlcNAc) epimerase polypeptide and an N-acetyl neuraminic acid condensing polypeptide.

13. The method of claim 12, wherein the GlcNAc epimerase polypeptide is a heterologous protein.

14. The method of claim 12, wherein the N-acetyl neuraminic acid condensing polypeptide is a heterologous protein.

15. The method of claim 11, wherein the enzymatic system for synthesizing sialic acid comprises a UDP-GlcNAc epimerase polypeptide and a sialate synthase polypeptide.

16. The method of claim 15, wherein the UDP-GlcNAc epimerase polypeptide or the sialate synthase polypeptide is a heterologous protein.

17. The method of claim 11, wherein the CMP-sialic acid synthase polypeptide is a heterologous protein.

18. The method of claim 11, wherein the sialyltransferase polypeptide is a heterologous protein.

19. The method of claim 11, wherein the acceptor substrate is a monosaccharide selected from the group consisting of glucose, galactose, lactose, and mannose.

20. The method of claim 11, wherein the acceptor saccharide is lactose and the sialylated product sugar is sialylactose.

21. The method of claim 11, wherein the culture medium further comprises pyruvate.

22. The method of claim 11, wherein the sialylated product is produced on a commercial scale.

23. The method of claim 11, wherein the bacterium further comprises a heterologous CTP synthetase polypeptide.

24. The method of claim 12, wherein the GlcNAc epimerase polypeptide is a heterologous protein, the N-acetyl neuraminic acid condensing polypeptide is a heterologous protein, the CMP-sialic acid synthase polypeptide is a heterologous protein, and the sialyltransferase polypeptide is a heterologous protein.
